(19) United States
(12) Patent Application Publication
(10) Pub. No.: US 2012/0252853 A1
(43) Pub. Date: Oct. 4, 2012

(54) Positive allosteric modulators of nicotinic acetylcholine receptor

(75) Inventors: Anette Graven Sams, Vaerlose (DK); Jørgen Eskildsen, Kobenhavn S (DK); Jesper Frank Bastlund, Bronshoj (DK)

(73) Assignee: H. Lundbeck A/S, Valby-Copenhagen (DK)

(21) Appl. No.: 13/434,884

(22) Filed: Mar. 30, 2012

Related U.S. Application Data
(60) Provisional application No. 61/470,565, filed on Apr. 1, 2011.

(51) Int. Cl. A61K 31/404; C07D 417/12; A61K 31/427; C07D 413/12; A61K 31/4245; A61P 25/18; A61P 25/28; A61P 25/00; A61P 25/14; A61P 25/16; A61P 25/08; A61P 3/10; A61P 3/00; A61P 29/00; A61P 7/00; C07D 405/12

(52) U.S. Cl. 514/364; 548/467; 514/414; 548/181; 514/365; 548/143; 548/131

(57) ABSTRACT

The present invention relates to compounds useful in therapy, to compositions comprising said compounds, and to methods of treating diseases comprising administration of said compounds. The compounds referred to are positive allosteric modulators (PAMs) of the nicotinic acetylcholine α7 receptor.

### CROSS-REFERENCE TO RELATED APPLICATIONS

The present application claims the benefit of priority under 35 U.S.C. §119(e) of U.S. Provisional Patent Application No. 61/470,565, filed Apr. 1, 2011 which is hereby incorporated by reference in its entirety.

### FIELD OF THE INVENTION

The present invention relates to compounds useful in therapy, to compositions comprising said compounds, and to methods of treating diseases comprising administration of said compounds. The compounds referred to are positive allosteric modulators (PAMs) of the nicotinic acetylcholine α7 receptor.

### BACKGROUND OF THE INVENTION

Nicotinic acetylcholine receptors (nAChRs) belong to the super family of ligand gated ionic channels, and gate the flow of cations including calcium. The nAChRs are endogenously activated by acetylcholine (ACh) and can be divided into nicotinic receptors of the neuromuscular junction and neuronal nicotinic receptors (NNRs). The NNRs are widely expressed throughout the central nervous system (CNS) and the peripheral nervous system (PNS). The NNRs have been suggested to play an important role in CNS function by modulating the release of many neurotransmitters, for example, ACh, norepinephrine, dopamine, serotonin, and GABA, among others, resulting in a wide range of physiological effects.

Seventeen subunits of nAChRs have been reported to date, which are identified as α2-α10, β1-β4, γ, δ and δ. From these subunits, nine subunits, α2 through α7 and β2 through β4, prominently exist in the mammalian brain. Many functionally distinct nAChR complexes exist, for example five α7 subunits can form a receptor as a homomeric functional pentamer or combinations of different subunits can form heteromeric receptors such as α4β2 and α3β4 receptors (Gotti, C. et al., Prog. Neurobiol. 2004, 74: 363-396; Gotti, C. et al. Biochemical Pharmacology, 2009, 78: 703-711)

The homomeric α7 receptor is one of the most abundant NNRs, along with α4β2 receptors, in the brain, wherein it is heavily expressed in the hippocampus, cortex, thalamic nuclei, ventral tegmental area and substantia nigra (Broad, L. M. et al., Drugs of the Future, 2007, 32(2): 161-170, Poorthuis R B, Biochem Pharmacol. 2009, 1; 78(7):668-76).

The role of α7 NNR in neuronal signalling has been actively investigated. The α7 NNRs have been demonstrated to regulate intemeuron excitability and modulate the release of excitatory as well as inhibitory neurotransmitters. In addition, α7 NNRs have been reported to be involved in neuroprotective effects in experimental models of cellular damage (Shimo-hama, S., Biol Pharm Bull. 2009, 32(3):332-6).

Studies have shown that α7 subunits, when expressed recombinant in-vitro, activate and desensitize rapidly, and exhibit relatively higher calcium permeability compared to other NNR combinations (Papke, R. L. et al., J Pharmacol Exp Ther. 2009, 329(2):791-807).

The NNRs, in general, are involved in various cognitive functions, such as learning, memory and attention, and therefore in CNS disorders, i.e., Alzheimer's disease (AD), Parkinson's disease (PD), attention deficit hyperactivity disorder (ADHD). Tourette's syndrome, schizophrenia, bipolar disorder, pain and tobacco dependence (Keller, J. J., et al., Behav. Brain Res. 2005, 162: 143-52; Haydar S, N. et al., Curr Top Med. Chem. 2010; 10(2):144-52).

The α7 NNRs in particular, have also been linked to cognitive disorders including, for example, ADHD, autism spectrum disorders, AD, mild cognitive impairment (MCI), age associated memory impairment (AAMI) senile dementia, frontotemporal lobar degeneration, HIV associated dementia (HAD), HIV associated cognitive impairment (HIV-CI), Pick's disease, dementia associated with Lewy bodies, cognitive impairment associated with Multiple Sclerosis, Vascular Dementia, cognitive impairment in Epilepsy, cognitive impairment associated with fragile X, cognitive impairment associated with Friedreich's Ataxia, and dementia associated with Down's syndrome, as well as cognitive impairment associated with schizophrenia. In addition, α7-NNRs have been shown to be involved in the neuroprotective effects of nicotine both in vitro (Jonnala, R. B. et al., J. Neurosci. Res., 2001, 66: 565-572) and in vivo (Shimohama, S. Brain Res., 1998, 779: 359-363) as well as in pain signalling. More particularly, neurodegeneration underlies several progressive CNS disorders, including, but not limited to, AD, PD, amyotrophic lateral sclerosis, Huntington's disease, dementia with Lewy bodies, as well as diminished CNS function resulting from traumatic brain injury. For example, the impaired function of α7 NNRs by beta-amyloid peptides linked to AD has been implicated as a key factor in development of the cognitive deficits associated with the disease (Liu, Q.-S., et al., PNAS, 2001, 98: 4734-4739). Thus, modulating the activity of α7 NNRs demonstrates promising potential to prevent or treat a variety of diseases indicated above, such as AD, other dementias, other neurodegenerative diseases, schizophrenia and neurodegeneration, with an underlying pathology that involves cognitive function including, for example, aspects of learning, memory, and attention (Thomsen, M. S. et al., Curr Pharm Des. 2010 January; 16(3):323-43; Olincy, A. et al. Arch Gen Psychiatry. 2006, 63(6):630-8; Deutsch, S. I., Clin Neuropharmacol. 2010, 33(3):114-20; Feuerbach, D., Neuropharmacology. 2009, 56(1): 254-63)

The NNR ligands, including α7 ligands, have also been implicated in weight control, diabetis inflammation, angiogenesis and as potential analgesics (Marrero, M. B. et al., J. Pharmacol Exp Ther. 2010, 332(1):173-80; Vincler, M., Exp. Opin. Invest. Drugs, 2005, 14 (10): 1191-1198; Rosas-Ballina, M., J. Intern Med. 2009, 265(6):663-79; Arias, H. R., Int. J. Biochem Cell Biol. 2009, 41(7):1441-51).

Nicotine is known to enhance attention and cognitive performance, reduced anxiety, enhanced sensory gating, and analgesia and neuroprotective effects when administered. Such effects are mediated by the non-selective effect of nicotine at multiple nicotinic receptor subtypes. However, nicotine also exerts adverse events, such as cardiovascular and gastrointestinal problems (Karaconji, I. B. et al., Arh Hig Rada Toksikol. 2005, 56(4):363-71). Consequently, there is a need to identify subtype-selective compounds that retain the beneficial effects of nicotine, or an NNR ligand, while eliminating or decreasing adverse effects.

Examples of reported NNR ligands are α7 NNR agonists, such as DMXB-A, SSR180711 and ABT-107, which have shown some beneficial effects on cognitive processing both in rodents and humans (H312: 1213-22; Olincy, A. et al., Arch Gen Psychiatry. 2006 63(6):630-8; Pichat, P., et al., Neuropsychopharmacology. 2007 32(1):17-34; Bitner, R. S., J Pharmacol Exp Ther. 2010 1; 334(3):875-86). In addition, modulation of α7 NNRs have been reported to improve negative symptoms in patients with schizophrenia (Freedman, R. et al., Am J Psychiatry. 2008 165(8):1040-7).

Despite the beneficial effects of NNR ligands, it remains uncertain whether chronic treatment with agonists affecting NNRs may provide suboptimal benefit due to sustained activation and desensitization of the NNRs, in particular the α7 NNR subtype. In contrast to agonists, administering a positive allosteric modulator (PAM) can reinforce endogenous cholinergic transmission without directly stimulating the target receptor. Nicotinic PAMs can selectively modulate the activity of ACh at NNRs, preserving the activation and deactivation kinetics of the receptor. Accordingly, α7 NNR-selective PAMs have emerged (Faghih, R., Recent Pat CNS Drug Discov. 2007, 2(2):99-106).

Consequently, it would be beneficial to increase α7 NNR function by enhancing the effect of the endogenous neurotransmitter acetylcholine via PAMs. This could reinforce the endogenous cholinergic neurotransmission without directly activating α7 NNRs, like agonists. Indeed, PAMs for enhancing channel activity have been proven clinically successful for GABAa receptors where benzodiazepines and barbiturates, behave as PAMs acting at distinct sites (Hevers, W. et al., Mol. Neurobiol., 1998, 18: 35-86).

To date, only a few NNR PAMs are known, such as 5-hydroxyindole (5-HI), ivermectin, galantamine, and SLURP-1, a peptide derived from acetylcholinesterase (AChE). Genistein, a kinase inhibitor was also reported to increase α7 responses. PNU-120596, a urea derivative, was reported to increase the potency ACh as well as improve auditory gating deficits induced by amphetamine in rats. Also, NS1738, JNJ-1930942 and compound 6 have been reported to potentiate the response of ACh and exert beneficial effect in experimental models of sensory and cognitive processing in rodents. Other NNR PAMs include derivatives of quinuclidine: indole, benzopyrazole, thiazole, and benzoisothiazoles (Hurst, R. S. et al., J. Neurosci. 2005, 25: 4396-4.405; Faghih, R., Recent Pat CNS Drug Discov. 2007, 2(2):99-106; Timmermann, D. B., J Pharmacol Exp Ther. 2007 October; 323(1):294-307; Ng, H. J. et al., Proc Natl Acad Sci USA. 2007, 104(19): 8059-64; Dinklo, T., J Pharmacol Exp Ther. 2011, 336(2): 560-74.).

Of particular examples WO 01/32619 discloses that compounds of the following core structure possess PAM activity

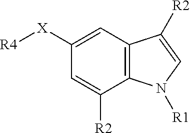

WO 2009/100294 discloses compounds with the core structure

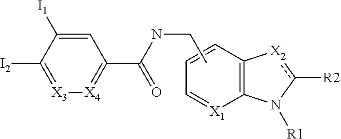

with PAM activity

WO 2009/127678 and WO 2010/119078 discloses compounds with the core structures

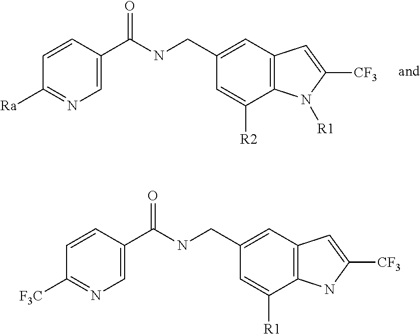

with PAM activity based on an in vitro assay. Likewise WO 2009/127679 discloses 25 compounds of similar structures with PAM activity.

The α7 NNR PAMs presently known generally demonstrate weak activity, have a range of non-specific effects, or can only achieve limited access to the central nervous system where α7 NNRs are abundantly expressed. Accordingly, it would be beneficial to identify and provide new PAM compounds of 07 NNRs and compositions for treating diseases and disorders wherein α7 NNRs are involved.

It would further be particularly beneficial if such compounds can provide improved efficacy of treatment while reducing adverse effects associated with compounds targeting neuronal nicotinic receptors by selectively modulating α7 NNRs. In particular, some compounds with α7 NNR PAM activity are found to be openers of voltage dependent Kv7 potassium channels, also termed KCNQ channels. KCNQ channel openers have been associated with a complex range of potential side-effects including but not limited to effects on, the cardiovascular system (Mackie A R, Mol Pharmacol. 2008 74(5):1171-9), bladder function (Gopalakrishnan M, Expert Opin Ther Targets. 2004 8(5):437-58) and hypothermia (Kristensen L, Neurosci Lett. 2011 20; 488(2):178-82). In addition, KCNO channel blockers have been suggested to have pro-cognition potential (Gribkoff V K, Expert Opin Ther Targets. 2003, 7(6):737-48), which points to a possible disruptive effect of KCNQ channel openers on cognition. There is therefore a need for new α7 NNR PAMs which show reduced effect as openers of KCNQ channels. Compounds with such improvements are likely to benefit from e.g. reduced side effects, enlarged therapeutic index, improved tolerability and improved compliance.

Another disadvantage of many known α7 NNR PAMs is low aqueous solubility which might result in low bioavailability and problems in relation to pharmaceutical formulation.

Consequently, the present invention discloses novel compounds that are PAMs of α7 NNRs. The compounds of the invention furthermore possess improved properties compared to known NNR PAMs. Furthermore, the invention discloses pharmaceutical compositions, methods of preparation and uses of said compounds.

The four compounds:
furan-2-carboxylic acid (2-methyl-1H-indol-5-ylmethyl)-amide;
furan-2-carboxylic acid (1,2-dimethyl-1H-indol-5-ylmethyl)-amide;
thiophene-2-carboxylic acid (1,2-dimethyl-1H-indol-5-ylmethyl)-amide;
thiophene-2-carboxylic acid (2-methyl-1H-indol-5-ylmethyl)-amide
   are published in the STN database and are not included in the compounds of the present invention.

### SUMMARY OF THE INVENTION

The objective of the present invention is to provide compounds that are positive allosteric modulators of the nicotinic acetylcholine receptor subtype α7.

A further objective of the present invention is to provide compounds which have such activity, and which have improved properties compared to known PAMs of the α7 NNR.

The compounds of the present invention are defined by formula [I] below:

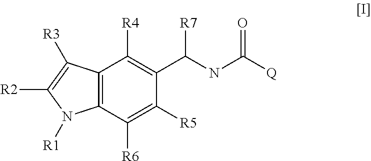

wherein R1 represents H, trifluoromethyl, difluoromethyl, C₁₋₄alkyl, C₂₋₄alkenyl or C₂₋₄alkynyl;
R2 represents H, C₁₋₄alkyl, C₂₋₄alkenyl, C₂₋₄alkynyl, halogen or cyano, wherein said C₁₋₄alkyl, C₂₋₄alkenyl or C₂₋₄alkynyl is optionally substituted with one or more substituents selected from chlorine and fluorine;
R3, R4, R5 and R6 are selected independently from H, C₂₋₄alkenyl, C₂₋₄alkynyl, halogen and cyano, wherein said C₁₋₄alkyl, C₂₋₄alkenyl or C₂₋₄alkynyl is optionally substituted with one or more substituents selected from chlorine and fluorine;
R7 represents H, methyl, trifluoromethyl or hydroxymethyl;
Q represents a heteroaryl with 5 ring atoms, wherein 1, 2 or 3 ring atoms are selected independently from O, N and S, wherein said heteroaryl may be optionally substituted on its carbon atoms with one or more substituents represented by R10, and provided that said heteroaryl cannot be 1,2,3 triazolyl or imidazolyl;
each R10 is independently selected from C₁₋₄alkyl, C₂₋₄alkenyl, C₂₋₄alkynyl, C₁₋₄alkoxy, C₁₋₄ fluoroalkoxy, halogen and oxo, wherein said C₁₋₄alkyl, C₂₋₄alkenyl or C₂₋₄alkynyl is optionally substituted with one or more substituents selected from fluorine, C₁₋₄alkoxy and C₁₋₄ fluoroalkoxy;
if one or more ring atoms in said heteroraryl are N atoms these may, when valency allows, individually be optionally substituted with a substituent represented by R11, wherein each R11 is independently selected from C₁₋₄alkyl and a monocyclic saturated ring moiety having 4-6 ring atoms wherein one of said ring atoms may be O and the rest is C, and wherein said C₁₋₄ alkyl may be optionally substituted with one or more substituents selected from fluorine, C₁₋₄alkoxy and C₁₋₄-fluoroalkoxy;
when Q is a pyrazolyl at least one of the N atoms in said pyrazolyl must be substituted with R11;
two R10 or one R10 and one R11 may, when sitting on neighbouring ring atoms and when represented by C₁₋₄alkyl be linked together by a carbon bond to form a fused ring system;
and pharmaceutically acceptable salts thereof;
with the proviso that the compound of formula [I] is other than
furan-2-carboxylic acid (2-methyl-1H-indol-5-ylmethyl)-amide;
furan-2-carboxylic acid (1,2-dimethyl-1H-indol-5-ylmethyl)-amide;
thiophene-2-carboxylic acid (1,2-dimethyl-1H-indol-5-ylmethyl)-amide;
thiophene-2-carboxylic acid (2-methyl-1H-indol-5-ylmethyl)-amide.

In one embodiment, the invention relates to a compound according to formula [I], and pharmaceutically acceptable salts thereof, for use in therapy.

In one embodiment, the invention relates to a compound according to formula [I], and pharmaceutically acceptable salts thereof, for use in the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); traumatic brain injury; epilepsy; post-traumatic stress; Wemicke-Korsakoff syndrome (WKS); post-traumatic amnesia; cognitive deficits associated with depression; diabetes, weight control, inflammatory disorders, reduced angiogenesis; amyotrophic lateral sclerosis and pain.

In one embodiment, the invention relates to a pharmaceutical composition comprising a compound according to formula [I] and pharmaceutically acceptable salts thereof, and one or more pharmaceutically acceptable carrier or excipient.

In one embodiment, the invention relates to a kit comprising a compound according to formula [I], and pharmaceutically acceptable salts thereof, together with a compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.

In one embodiment, the invention relates to a method for the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain, which method comprises the administration of a therapeutically effective amount of a compound according to formula [I], and pharmaceutically acceptable salts thereof.

In one embodiment, the invention relates to the use of a compound according to formula [I], and pharmaceutically acceptable salts thereof, for the manufacture of a medicament for the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.

### DEFINITIONS

In the present context, “optionally substituted” means that the indicated moiety may or may not be substituted, and when substituted is mono-, di-, or tri-substituted, such as with 1, 2 or 3 substituents. In some instances, the substituent is independently selected from the group consisting of C₁₋₄alkyl, C₂₋₄alkenyl, C₂₋₄alkynyl, phenyl, C₁₋₄alkoxy, hydroxy, halogen and oxo. It is understood that where no substituents are indicated for an “optionally substituted” moiety, then the position is held by a hydrogen atom.

In the present context, “alkyl” is intended to indicate a straight, branched and/or cyclic saturated hydrocarbon. In particular “C₁₋₄alkyl” is intended to indicate such hydrocarbon having 1, 2, 3 or 4 carbon atoms. Examples of C₁₋₄alkyl include methyl, ethyl, propyl, butyl, cyclopropyl, cyclobutyl, methylcyclopropyl, 2-methyl-propyl and tert-butyl.

In the present context, “alkenyl” is intended to indicate a non-aromatic, straight, branched and/or cyclic hydrocarbon comprising at least one carbon-carbon double bond. In particular “C₂₋₄alkenyl” is intended to indicate such hydrocarbon having 2, 3 or 4 carbon atoms. Examples of C₂₋₄alkenyl include ethenyl, 1-propenyl, 2-propenyl, 1-butenyl, 2-butenyl and 3-butenyl.

In the present context, “alkynyl” is intended to indicate a non-aromatic, straight, branched and/or cyclic hydrocarbon comprising at least one carbon-carbon triple bond and optionally also one or more carbon-carbon double bonds. In particular “C₂₋₄alkynyl” is intended to indicate such hydrocarbon having 2, 3 or 4 carbon atoms. Examples of C₂₋₄alkynyl include ethynyl, 1-propynyl, 2-propynyl, 1-butynyl, 2-butynyl and 3-butynyl.

In the present context, “hydroxy” is intended to indicate —OH.

In the present context, “alkoxy” is intended to indicate a moiety of the formula —OR′, wherein R′ indicates alkyl as defined above. In particular “C₁₋₄alkoxy” is intended to indicate such moiety wherein the alkyl part has 1, 2, 3 or 4 carbon atoms. Examples of “C₁₋₄alkoxy” include methoxy, ethoxy, n-butoxy and tert-butoxy.

In the present context, “fluoroalkoxy” indicates an alkoxy as defined above substituted with one or more fluorine atoms per carbon atom. Examples include trifluoromethoxy and 2,2,2-trifluoroethoxy.

In the present context, the terms “halo” and “halogen” are used interchangeably and refer to fluorine, chlorine, bromine or iodine.

In the present context, the term “cyano” indicates the group —C≡N, which consists of a carbon atom triple-bonded to a nitrogen atom.

In the present context, a “5 membered heteroaryl” is intended to indicate a 5 membered ring wherein 1, 2 or 3 ring atoms are selected from O, N or S. Examples of 5 membered heteroaryls of the present invention include thiophenyl, pyrrolyl, furanyl, oxazotyl, isoxazolyl, thiazolyl, isothiazotyl, pyrazolyl, oxadiazolyl, thiadiazolyl.

In the present context, a “monocyclic moiety” is intended to indicate a ring formed structure comprising only one ring.

In the present context, a “fused ring system” is intended to indicate a system of monocyclic rings that share their connecting bonds. Said monocyclic rings can be aromatic or non-aromatic and they can comprise one or more atoms selected from N, O and S. One example of a “fused ring system” of the present invention is 5,6-Dihydro-4H-pyrrolo[1,2-b]pyrazole.

In the present context, “ring atom” is intended to indicate the atoms constituting a ring, and ring atoms are selected from C, N, O and S. As an example, benzene and toluene both have 6 carbons as ring atoms whereas pyridine has 5 carbons and 1 nitrogen as ring atoms.

In the present context, “heteroatom” means a nitrogen, oxygen or sulfur atom. In the present context, “oxo” is intended to indicate an oxygen substituent of a carbon atom that results in the formation of a carbonyl group (C═O). An oxo group that is a substituent of a nonaromatic carbon atom results in a conversion of —CH₂— to —C(═O)—. An oxo group that is a substituent of an aromatic carbon atom results in a conversion of —CH— to —C(═O)— and may result in a loss of aromaticity. Examples of 5 membered heteroaromats substituted with an oxo include 2,4-Dihydro-pyrazol-3-one, 3H-Thiazol-2-one, 4H-Isothiazol-5-one, 3H-Oxazol-2-one, 4H-Isoxazol-5-one.

In the present context, “deuterium” indicates the atomic isotope of hydrogen consisting of one proton and one neutron in its nucleus, and thus having an approximate weight of two (2). Deuterium is represented as D or ²H. An example of a substituent labeled with deuterium is trideuteriomethyl (MeD₃) wherein the three hydrogens in methyl are the ²H isotopes.

In the present context, pharmaceutically acceptable salts include pharmaceutical acceptable acid addition salts, pharmaceutically acceptable metal salts, ammonium and alkylated ammonium salts. Acid addition salts include salts of inorganic acids as well as organic acids.

Examples of suitable inorganic acids include hydrochloric, hydrobromic, hydroiodic, phosphoric, sulfuric, sulfamic, nitric acids and the like.

Examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in Berge, S. M. et al., J. Pharm. Sci. 1977, 66, 2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

In the present context, pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier may include any sustained release material known in the art, such as glyceryl monostearate or glyceryl distearate, alone or mixed with a wax.

In the present context, the term “therapeutically effective amount” of a compound means an amount sufficient to cure, alleviate or partially arrest the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

In the present context, the term “treatment” and “treating” means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatments are two separate aspects of the present invention. The patient to be treated is preferably a mammal, in particular a human being.

In the present context, the term “cognitive disorders” is intended to indicate disorders characterized by abnormalities in aspects of perception, problem solving, language, learning, working memory, memory, social recognition, attention and pre-attentional processing, such as by not limited to Attention Deficit Hyperactivity Disorder (ADHD), autism spectrum disorders, Alzheimer's disease (AD), mild cognitive impairment (MCI), age associated memory impairment (AAMI), senile dementia, vascular dementia, frontotemporal lobe dementia, Pick's disease, dementia associated with Lewy bodies, and dementia associated with Down's syndrome, cognitive impairment associated with Multiple Sclerosis, cognitive impairment in epilepsy, cognitive impairment associated with fragile X, cognitive impairment associated with neurofibromatosis, cognitive impairment associated with Friedreich's Ataxia, progressive supranuclear palsy (PSP), HIV associated dementia (HAD), HIV associated cognitive impairment (HIV-CI), Huntington's Disease, Parkinson's disease (PD), traumatic brain injury, epilepsy, post-traumatic stress, Wernicke-Korsakoff syndrome (WKS), post-traumatic amnesia, cognitive deficits associated with depression as well as cognitive impairment associated with schizophrenia.

In the present context, the term “autism spectrum disorders” is intended to indicate disorders characterized by widespread abnormalities of social interactions and verbal and non-verbal communication, as well as restricted interests, repetitive behavior and attention, such as by not limited to autism, Asperger syndrome, Pervasive Developmental Disorder Not Otherwise Specified (PDD-NOS), Rett syndrome, Angelmann syndrome, fragile X, DiGeorge syndrome and Childhood Disintegrative Disorder.

In the present context, the term “inflammatory disorders” is intended to indicate disorders characterized by abnormalities in the immune system such as by not limited to, allergic reactions and myopathies resulting in abnormal inflammation as well as non-immune diseases with etiological origins in inflammatory processes are thought to include by not be limited to cancer, atherosclerosis, osteoarthritis, rheumatoid arthritis and ischaemic heart disease.

### DETAILED DESCRIPTION OF THE INVENTION

The present inventors have found that certain new compounds are positive allosteric modulators (PAMs) of NNRs, and as such may be used in the treatment of various diseases and disorders.

PAMs of NNRs may be dosed in combination with other drugs in order to achieve more efficacious treatment in certain patient populations. An α7 NNR PAM may act synergistically with another drug, this has been described in animals for the combination of compounds affecting nicotinic receptors, including α7 NNRs and D2 antagonism (Wiker, C., Int J Neuropsychopharmacol. 2008 September; 11(6):845-50).

Thus, compounds of the present invention may be useful treatment in the combination with e.g. acetylcholinesterase inhibitors, glutamate receptor antagonists, dopamine transport inhibitors, noradrenalin transport inhibitors, D2 antagonists, D2 partial agonists, PDE10 antagonists, 5-HT2A antagonists, 5-HT6 antagonists and KCNQ antagonists, lithium, sodium channel blockers, GABA signalling enhancers.

In one embodiment, compounds of the present invention are used for treatment of subjects who are already in treatment with another drug. In one embodiment, compounds of the present invention are used as the sole medicament in treatment of a subject. In one embodiment, compounds of the present invention are used for treatment of subjects who are not already in treatment with another drug. In one embodiment, compounds of the present invention are adapted for administration simultaneous with another drug. In one embodiment compounds of the present invention are adapted for administration sequentially with another drug.

### Embodiments According to the Invention

In the following, embodiments of the invention are disclosed. The first embodiment is denoted E1, the second embodiment is denoted E2 and so forth.

E1. A compound according to formula I

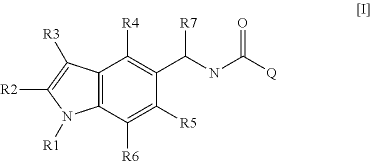

wherein R1 represents H, trifluoromethyl, difluoromethyl, C₂₋₄alkenyl or C₂₋₄alkynyl;
R2 represents H, C₁₋₄alkyl, C₂₋₄alkenyl, C₂₋₄alkynyl, halogen or cyano, wherein said C₁₋₄alkyl, C₂₋₄alkenyl or C₂₋₄alkynyl is optionally substituted with one or more substituents selected from chlorine and fluorine;
R3, R4, R5 and R6 are selected independently from H, C₂₋₄alkenyl, C₂₋₄alkynyl, halogen and cyano, wherein said C₁₋₄alkyl, C₂₋₄alkenyl or C₂₋₄alkynyl is optionally substituted with one or more substituents selected from chlorine and fluorine;
R7 represents H, methyl, trifluoromethyl or hydroxymethyl;
Q represents a heteroaryl with 5 ring atoms, wherein 1, 2 or 3 ring atoms are selected independently from O, N and S, wherein said heteroaryl may be optionally substituted on its carbon atoms with one or more substituents represented by R10, and provided that said heteroaryl cannot be 1,2,3 triazolyl or imidazolyl;
each R10 is independently selected from C₁₋₄alkyl, C₂₋₄alkenyl, C₂₋₄alkynyl, C₁₋₄alkoxy, C₁₋₄fluoroalkoxy, halogen and oxo, wherein said C₁₋₄alkyl, C₂₋₄alkenyl or C₂₋₄alkynyl is optionally substituted with one or more substituents selected from fluorine, C₁₋₄alkoxy and C₁₋₄ fluoroalkoxy;
if one or more ring atoms in said heteroraryl are N atoms these may, when valency allows, individually be optionally substituted with a substituent represented by R11, wherein each R11 is independently selected from C₁₋₄alkyl and a monocyclic saturated ring moiety having 4-6 ring atoms wherein one of said ring atoms may be 0 and the rest is C, and wherein said C₁₋₄ alkyl may be optionally substituted with one or more substituents selected from fluorine, C₁₋₄alkoxy and C₁₋₄fluoroalkoxy;
when Q is a pyrazolyl at least one of the N atoms in said pyrazolyl must be substituted with R11;
two R10 or one R10 and one R11 may, when sitting on neighbouring ring atoms and when represented by C₁₋₄alkyl be linked together by a carbon bond to form a fused ring system:
and pharmaceutically acceptable salts thereof;
with the proviso that the compound of formula [I] is other than
furan-2-carboxylic acid (2-methyl-1H-indol-5-ylmethyl)-amide;
furan-2-carboxylic acid (1,2-dimethyl-1H-indol-5-ylmethyl)-amide;
thiophene-2-carboxylic acid (1,2-dimethyl-1H-indol-5-ylmethyl)-amide;
thiophene-2-carboxylic acid (2-methyl-1H-indol-5-ylmethyl)-amide.
   E2. The compound according to embodiment 1, wherein R1 represents H, trifluoromethyl, difluoromethyl or C₁₋₂alkyl;
   R2 represents H, C₁₋₂alkyl or cyano, wherein said C₁₋₂alkyl is optionally substituted with one or more fluorine;
   R3, R4, R5 and R6 are selected independently from H, methyl and fluorine;
   R7 represents H, methyl or trifluoromethyl;
   Q represents a heteroaryl with 5 ring atoms, wherein 1, 2 or 3 ring atoms are selected independently from O, N and S, wherein said heteroaryl may be optionally substituted on ifs carbon atoms with one or more substituents represented by R10 and provided that said heteroaryl cannot be 1,2,3 triazolyl or imidazolyl;
   each R10 is independently selected from C₁₋₄alkyl, C₁₋₂alkoxy, halogen and oxo, wherein said C₁₋₄alkyl is optionally substituted with one or more substituents selected from fluorine and C₁₋₂alkoxy;
   if one or more ring atoms in said heteroraryl are N atoms these may, when valency allows, individually be optionally substituted with a substituent represented by R11, wherein each R11 is independently selected from C₁₋₄alkyl and a monocyclic saturated ring moiety having 4-6 ring atoms, wherein one of said ring atoms may be O and the rest is C, and wherein said C₁₋₄alkyl may be optionally substituted with one or more substituents selected from fluorine and C₁₋₂alkoxy;
   when Q is a pyrazolyl at least one of the N atoms in said pyrazolyl must be substituted with R11;
   two R10 or one R10 and one R11 may, when sitting on neighbouring ring atoms and when both are represented by C₁₋₄alkyl be linked together by a carbon bond to form a fused ring system.
   E3. The compound according to any of embodiments 1-2, wherein Q is selected from optionally substituted thiophenyl, pyrrolyl, furanyl, oxazolyl, isoxazolyl, thiazolyl, isothiazolyl, pyrazolyl, [1,2,4]oxadiazolyl and [1,3,4]oxadiazolyl.
   E4. The compound according to any of embodiments 1-3, wherein R1 is selected from H, methyl, trifluoromethyl or difluoromethyl.
   E5. The compound according to embodiment 4, wherein R1 is H.
   E6. The compound according to any of embodiments 1-5, wherein four or more of R2, R3, R4, R5 and R6 are H.
   E7. The compound according to embodiment 6, wherein all of R3, R4, R5 and R6 are H.
   E8. The compound according to any of embodiments 1-6, wherein R3. R4, R5 and R6 are selected independently from H, methyl and fluorine.
   E9. The compound according to any of embodiments 1-8, wherein R2 is selected from H, methyl, trifluoromethyl, difluoromethyl, [2,2,2]-trifluoroethyl and cyano.
   E10. The compound according to embodiment 9, wherein R2 is methyl.
   E11. The compound according to embodiment 10, wherein R2 is MeD₃.
   E12. The compound according to any of embodiments 1-11, wherein R3 is H
   E13. The compound according to any of embodiments 1-12, wherein R7 is selected from H, methyl or trifluoromethyl
   E14. The compound according to embodiment 13, wherein R7 is methyl.
   E15. The compound according to any of embodiments 1-14, wherein Q is optionally substituted thiophenyl.
   E16. The compound according to any of embodiments 1-14, wherein Q is optionally substituted pyrrolyl.
   E17. The compound according to any of embodiments 1-14, wherein Q is optionally substituted furanyl.
   E18. The compound according to any of embodiments 1-14, wherein Q is optionally substituted oxazolyl.
   E19. The compound according to any of embodiments 1-14, wherein Q is optionally substituted isoxazolyl.
   E20. The compound according to any of embodiments 1-14, wherein Q is optionally substituted thiazolyl.
   E21. The compound according to any of embodiments 1-14, wherein Q is optionally substituted pyrazolyl, wherein at least one of the N atoms in said pyrazolyl is substituted with R11.
   E22. The compound according to any of embodiments 1-14, wherein Q is optionally substituted [1,2,4]oxadiazolyl.
   E23. The compound according to any of embodiments 1-14, wherein Q is optionally substituted [1,3,4]oxadiazolyl.
   E24. The compound according to any of embodiments 1-23, wherein each R10 is independently selected from C₁₋₄alkyl, C₁₋₂alkoxy, halogen and oxo, wherein said C₁₋₄alkyl is optionally substituted with one or more substituents selected from fluorine and C₁₋₂alkoxy.
   E25. The compound according to embodiment 24, wherein each R10 is independently selected from methyl, isopropyl, methoxy and oxo.
   E26. The compound according to any of embodiments 1-25, wherein each R11 is independently selected from C₁₋₄alkyl and a monocyclic saturated ring moiety having 4-6 ring atoms wherein one of said ring atoms may be 0 and the rest is C, and wherein said C₁₋₄alkyl may be optionally substituted with one or more substituents selected from fluorine and C₁₋₂alkoxy.
   E27. The compound according to embodiment 26, wherein each R11 is independently selected from methyl, trifluoromethyl and [2,2,2]-trifluoroethyl.
   E28. The compound according to any of embodiments 1-2, wherein R1 represents H; R2 represents H or methyl; R3-R6 are selected independently from H and fluorine, and R7 represents H or methyl.
   E29. The compound according to any of embodiments 1-28, with the proviso that R2 is not trifluoromethyl.
   E30. The compound according to any of the embodiments 1-29, wherein R7 is not H and said compound is essentially the enantiomer as depicted in formula I′

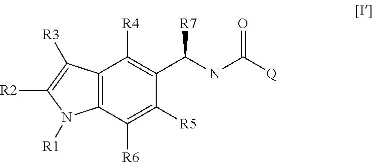

E31. The compound according to embodiment 1 selected from
1: 3-methyl-N-[(2-methyl-1H-indol-5-yl)methyl]furan-2-carboxamide
2: 2,4-dimethyl-N-[(2-methyl-1H-indol-5-yl)methyl]thiazole-5-carboxamide
3: 5-Chloro-thiophene-2-carboxylic acid (2-methyl-1H-indol-5-ylmethyl)-amide
4: 3-chloro-N-[(2-methyl-1H-indol-5-yl)methyl]thiophene-2-carboxamide
5: N-[(2-methyl-1H-indol-5-yl)methyl]thiazole-4-carboxamide
6: N-[(2-methyl-1H-indol-5-yl)methyl]thiophene-3-carboxamide
7: 2,5-dimethyl-N-[(2-methyl-1H-indol-5-yl)methyl]furan-3-carboxamide
8: 5-methyl-N-[(2-methyl-1H-indol-5-yl)methyl]isoxazole-3-carboxamide
9: 3-methyl-N-[(2-methyl-1H-indol-5-yl)methyl]thiophene-2-carboxamide
10: N-[(2-methyl-1H-indol-5-yl)methyl]furan-2-carboxamide
11: N-[(2-methyl-1H-indol-5-yl)methyl]furan-3-carboxamide
12: 1,5-dimethyl-N-[(2-methyl-1H-indol-5-yl)methyl]pyrazole-3-carboxamide
13: N-[(2-methyl-1H-indol-5-yl)methyl]thiophene-2-carboxamide
14: N-[(2-methyl-1H-indol-5-yl)methyl]-1H-pyrrole-2-carboxamide
15: 4-methyl-N-[(2-methyl-1H-indol-5-yl)methyl]isoxazole-5-carboxamide
16: 4-methyl-N-[1-(2-methyl-1H-indol-5-yl)ethyl]isoxazole-5-carboxamide
17: 3-methyl-N-[(3-methyl-1H-indol-5-yl)methyl]isoxazole-5-carboxamide
18: 3-methoxy-N-[1-(2-methyl-1H-indol-5H)ethyl]isoxazole-5-carboxamide
19: 3-methoxy-N-[(2-methyl-1H-indol-5-yl)methyl]isoxazole-5-carboxamide
20: N-[(2-methyl-1H-indol-5-yl)methyl]thiazole-2-carboxamide
21: 2,4-dimethyl-N-[(2-methyl-1H-indol-5-yl)methyl]oxazole-5-carboxamide
22: 4-methyl-N-[(2-methyl-1H-indol-5-yl)methyl]oxazole-5-carboxamide
23: 3-methyl-N-[(7-methyl-1H-indol-5-yl)methyl]isoxazole-5-carboxamide
24: 5-methyl-N-[(2-methyl-1H-indol-5-yl)methyl]thiophene-2-carboxamide
25: 2-methyl-N-[(2-methyl-1H-indol-5-yl)methyl]thiazole-4-carboxamide
26: N-[(2-methyl-1H-indol-5-yl)methyl]isoxazole-5-carboxamide
27: 3,5-dimethyl-N-[(2-methyl-1H-indol-5-yl)methyl]isoxazole-4-carboxamide
28: 3-methyl-N-[(2-methyl-1H-indol-5-yl)methyl]isoxazole-5-carboxamide
29: 1-methyl-N-[(2-methyl-1H-indol-5-yl)methyl]pyrazole-4-carboxamide
30: 1-methyl-N-[[2-(trifluoromethyl)-1H-indol-5-yl]methyl]pyrazole-4-carboxamide
31: N-[[2-(trifluoromethyl)-1H-indol-5-yl]methyl]isoxazole-5-carboxamide
32: 2-methyl-N-[(2-methyl-1H-indol-5-yl)methyl]-3-oxo-1H-pyrazole-5-carboxamide
33: 3-methyl-N-[1-(2-methyl-1H-indol-5-yl)ethyl]isoxazole-5-carboxamide
34: 3-isopropyl-N-[(2-methyl-1H-indol-5-yl)methyl]isoxazole-5-carboxamide
35: 2-methyl-N-[(2-methyl-1H-indol-5-yl)methyl]thiazole-5-carboxamide
36: N-[(2-methyl-1H-indol-5-yl)methyl]-1-(2,2,2-trifluoroethyl)pyrazole-4-carboxamide
37: 1-methyl-N-[(2-methyl-1H-indol-5-yl)methyl]pyrrole-3-carboxamide 38: N-[1-(1H-indol-5-yl)ethyl]-3-methyl-isoxazole-5-carboxamide
39: 3-methyl-N-[(6-methyl-1H-indol-5-yl)methyl]isoxazole-5-carboxamide
40: 1-(2,2,2-trifluoroethyl)-N-[[2-(trifluoromethyl)-1H-indol-5-yl]methyl]pyrazole-4-carboxamide
41: N-[(2,3-dimethyl-1H-indol-5H)methyl]-3-methyl-isoxazole-5-carboxamide
42: 2-methyl-N-[(1R)-1-(2-methyl-1H-indol-5-yl)ethyl]thiazole-5-carboxamide
43: 1-ethyl-N-[(1R)-1-(2-methyl-1H-indol-5-yl)ethyl]pyrazole-4-carboxamide
44: 1-methyl-N-[(1R)-1-(2-methyl-1H-indol-5-yl)ethyl]pyrazole-4-carboxamide
45: 3-methyl-N-[(1S)-2,2,2-trifluoro-1-(2-methyl-1H-indol-5-yl)ethyl]isoxazole-5-carboxamide
46: 1-(2-methoxyethyl)-N-[(1R)-1-(2-methyl-1H-indol-5-yl)ethyl]pyrazole-4-carboxamide
47: 3-methyl-N-[(1R)-1-(2-methyl-1H-indol-5-yl)ethyl]isothiazole-5-carboxamide
48: 1-(2-fluoroethyl)-N-[(1R)-1-(2-methyl-1H-indol-5-yl)ethyl]pyrazole-4-carboxamide
49: N-[(2-methyl-1H-indol-5-yl)methyl]-1-(oxetan-3-yl)pyrazole-4-carboxamide
50: 2-methyl-N-[(2-methyl-1H-indol-5-yl)methyl]oxazole-5-carboxamide 51: N-[(2-methyl-1H-indol-5-yl)methyl]-1-tetrahydrofuran-3-yl-pyrazole-4-carboxamide
52: N-[(1R)-1-(2-methyl-1H-indol-5-yl)ethyl]-1-(oxetan-3-yl)pyrazole-4-carboxamide
53: N-[(1R)-1-(4-fluoro-2-methyl-1H-indol-5-yl)ethyl]-3-methyl-isoxazole-5-carboxamide
54: N-[(1S)-1-(4-fluoro-2-methyl-1H-indol-5-yl)ethyl]-3-methyl-isoxazole-5-carboxamide
55: 2,5-dimethyl-N-[(2-methyl-1H-indol-5-yl)methyl]pyrazole-3-carboxamide
56: 4-chloro-3-methyl-N-[(2-methyl-1H-indol-5-yl)methyl]isoxazole-5-carboxamide
57: 1-methyl-N-[(2-methyl-1H-indol-5-yl)methyl]pyrrole-2-carboxamide
58: N-(1H-indol-5-ylmethyl)-3-methyl-isoxazole-5-carboxamide
59: N-(1H-indol-5-ylmethyl)-3,4-dimethyl-2-oxo-thiazole-5-carboxamide
60: 3,4-dimethyl-N-[(2-methyl-1H-indol-5-yl)methyl]-2-oxo-thiazole-5-carboxamide
61: 3-methyl-N-[(1R)-1-(2-methyl-1H-indol-5-yl)ethyl]isoxazole-5-carboxamide
62: 3-methyl-N-[(1S)-1-(2-methyl-1H-indol-5-yl)ethyl]isoxazole-5-carboxamide
63: N-[[1-(difluoromethyl)indol-5-yl]methyl]-3-methyl-isoxazole-5-carboxamide
64: N-[[1-(difluoromethyl)-2-methyl-indol-5-yl]methyl]-3-methyl-isoxazole-5-carboxamide
65: 3-methyl-N-([2-(trifluoromethyl)-1H-indol-5-yl)methyl]isoxazole-5-carboxamide
66: 2-methyl-N-[[2-(trifluoromethyl)-1H-indol-5-yl]methyl]thiazole-5-carboxamide
67: 3-isopropyl-N-[[2-(trifluoromethyl)-1H-indol-5-yl]methyl]isoxazole-5-carboxamide
68: N-[[1-(difluoromethyl)indol-5-yl]methyl]-1-(2,2,2-trifluoroethyl)pyrazole-4-carboxamide
69: N-[[1-(difluoromethyl)-2-methyl-indol-5-yl]methyl]-1-(2,2,2-trifluoroethyl)pyrazole-4-carboxamide
70: N-[[1-(difluoromethyl)-2-methyl-indol-5-yl]methyl]-3-(2,2,2-trifluoroethyl)isoxazole-5-carboxamide
71: 1-isopropyl-N-[(2-methyl-1H-indol-5-yl)methyl]pyrazole-4-carboxamide
72: N-[[1-(difluoromethyl)indol-5-yl]methyl]-3-(2,2,2-trifluoroethyl)isoxazole-5-carboxamide
73: 3-methyl-N-[[2-(trideuteriomethyl)-1H-indol-5-yl]methyl]isoxazole-5-carboxamide
74: 5-methyl-N-[(2-methyl-1H-indol-5-yl)methyl]-1,3,4-oxadiazole-2-carboxamide
75: 5-ethyl-N-[(2-methyl-1H-indol-5-yl)methyl]isoxazole-3-carboxamide
76: 2-methyl-N-[(2-methyl-1H-indol-5-yl)methyl]oxazole-4-carboxamide
77: 5-methyl-N-[(2-methyl-1H-indol-5-yl)methyl]-1,2,4-oxadiazole-3-carboxamide
78: N-[[2-(trideuteriomethyl)-1H-indol-5-yl]methyl]-3-(2,2,2-trifluoroethyl)isoxazole-5-carboxamide
79: N-[(2-methyl-1H-indol-5-yl)methyl]-3-(2,2,2-trifluoroethyl)isoxazole-5-carboxamide
80: 3-methyl-N-[(1-(trifluoromethyl)indol-5-yl)methyl]isoxazole-5-carboxamide
81: 1-(2,2,2-trifluoroethyl)-N-[[1-(trifluoromethyl)indol-5-yl)]methyl]pyrazole-4-carboxamide
82: N-[[2-(trideuteriomethyl)-1H-indol-5-yl]methyl]-1-(2,2,2-trifluoroethyl)pyrazole-4-carboxamide
83: N-[[2-methyl-1-(trifluoromethyl)indol-5-yl]methyl]-1-(2,2,2-trifluoroethyl)pyrazole-4-carboxamide
84: 3-methyl-N-[[2-methyl-1-(trifluoromethyl)indol-5-yl]methyl]isoxazole-5-carboxamide
85: N-[[2-methyl-1-(trifluoromethyl)indol-5-yl]methyl]-3-(2,2,2-trifluoroethyl)isoxazole-5-carboxamide
86: N-[(1R)-1-(2-methyl-1H-indol-5-yl)ethyl]-1-(2,2,2-trifluoroethyl)pyrazole-4-carboxamide
87: 5-tert-butyl-N-[(2-methyl-1H-indol-5-yl)methyl]isoxazole-3-carboxamide
88: N-[[1-methyl-2-(trifluoromethyl)indol-5-yl]methyl]-1-(2,2,2-trifluoroethyl)pyrazole-4-carboxamide
89: 3-methyl-N-[[1-methyl-2-(trifluoromethyl)indol-5-yl]methyl]isoxazole-5-carboxamide
90: N-[[1-methyl-2-(trifluoromethyl)indol-5-yl]methyl]-3-(2,2,2-trifluoroethyl)isoxazole-5-carboxamide
91: N-[[2-(difluoromethyl)-1H-indol-5-yl]methyl]-1-(2,2,2-trifluoroethyl)pyrazole-4-carboxamide
92: N-[[2-(difluoromethyl)-1H-indol-5-yl]methyl]-3-methyl-isoxazole-5-carboxamide
93: N-[(2-(difluoromethyl)-1H-indol-5-yl)methyl]-3-(2,2,2-trifluoroethyl)isoxazole-5-carboxamide
94: 1-(2-fluoroethyl)-N-[(2-methyl-1H-indol-5-yl)methyl]pyrazole-3-carboxamide
95: 1-(2-methoxyethyl)-N-[(2-methyl-1H-indol-5-yl)methyl]pyrazole-3-carboxamide
96: 3-methyl-N-[1-[2-(trifluoromethyl)-1H-indol-5-yl]ethyl]isoxazole-5-carboxamide
97: 3-methyl-N-[1-[2-(trifluoromethyl)-1H-indol-5-yl]ethyl]isoxazole-5-carboxamide
98: 3-(methoxymethyl)-N-[(2-methyl-1H-indol-5-yl)methyl]isoxazole-5-carboxamide
99: 1-(2-fluoroethyl)-N-[(2-methyl-1H-indol-5-yl)methyl]pyrazole-4-carboxamide
100: 1-(2-methoxyethyl)-N-[(2-methyl-1H-indol-5-yl)methyl]pyrazole-4-carboxamide
101: N-[(2,3-dimethyl-1H-indol-5-yl)methyl]-1-(2,2,2-trifluoroethyl)pyrazole-4-carboxamide
102: N-[(1R)-1-(6-fluoro-2-methyl-1H-indol-5-yl)ethyl]-3-methyl-isoxazole-5-carboxamide
103: N-[(1R)-1-(6-fluoro-7-methyl-1H-indol-5H)ethyl]-3-methyl-isoxazole-5-carboxamide
104: N-[(6-fluoro-2-methyl-1H-indol-5-yl)methyl]-3-methyl-isoxazole-5-carboxamide
105: N-[(6-fluoro-7-methyl-1H-indol-5-yl)methyl]-3-methyl-isoxazole-5-carboxamide
106: 3-(2-fluoroethyl)-N-[(2-methyl-1H-indol-5-yl)methyl]isoxazole-5-carboxamide
107: 3-methyl-N-[(2-methyl-1H-indol-5-yl)methyl]isothiazole-5-carboxamide
108: 3-methyl-N-[(2-(2,2,2-trifluoroethyl)-1H-indol-5-yl)methyl]isoxazole-5-carboxamide
109: N-[(7-fluoro-2-methyl-1H-indol-5-yl)methyl]-3-methyl-isoxazole-5-carboxamide
110: N-[(4-fluoro-2-methyl-1H-indol-5-yl)methyl]-3-methyl-isoxazole-5-carboxamide
111: N-[(1R)-1-(7-fluoro-2-methyl-1H-indol-5-yl)ethyl]-3-methyl-isoxazole-5-carboxamide
112: 3-(methoxymethyl)-N-[(1R)-1-(2-methyl-1H-indol-5-yl)ethyl]isoxazole-5-carboxamide
113: 3-(2-fluoroethyl)-N-[(1R)-1-(2-methyl-1H-indol-5-yl)ethyl]isoxazole-5-carboxamide
114: 3-(methoxymethyl)-N-[[2-(trifluoromethyl)-1H-indol-5-yl]methyl]isoxazole-5-carboxamide
115: 3-(2-fluoroethyl)-N-(1H-indol-5-ylmethyl)isoxazole-5-carboxamide
116: 5-methyl-N-[(2-methyl-1H-indol-5-yl)methyl]oxazole-2-carboxamide
117: N-[(2-methyl-1H-indol-5-yl)methyl]-5,6-dihydro-4H-pyrrolo[1,2-b]pyrazole-3-carboxamide
118: N-[(2-methyl-1H-indol-5-yl)methyl]-5,6-dihydro-4H-pyrrolo[1,2-b]pyrazole-2-carboxamide
119: 3-methyl-N-[(2-methyl-1H-indol-5-yl)methyl]-1,2,4-oxadiazole-5-carboxamide
120: N-[(2-cyano-1H-indol-5-yl)methyl]-3-methyl-isothiazole-5-carboxamide
121: N-[(1R)-1-(2-cyano-1H-indol-5-yl)ethyl]-3-methyl-isoxazole-5-carboxamide;
   and pharmaceutically acceptable salts of any of these compounds.
   E32. A compound according to any of embodiments 1-31, for use in therapy.
   E33. A compound according to any of embodiments 1-31, for use in the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; demehtia associated with Down's syndrome; Huntington's Disease; Parkinson's disease (PD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.
   E34. The compound according to embodiment 33, wherein said a disease or disorder is selected from schizophrenia; AD; ADHD; autism spectrum disorders; PD; amyotrophic lateral sclerosis; Huntington's disease; dementia associated with Lewy bodies and pain.
   E35. The compound according to embodiment 34, wherein said disease or disorder is selected from schizophrenia; AD; ADHD and autism spectrum disorders.
   E36. The compound according to embodiment 35, wherein said disease or disorder is selected from negative and/or cognitive symptoms of schizophrenia.
   E37. The compound according to any of embodiments 33-35, wherein the treatment further comprises treatment with a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E38. The compound according to embodiment 37, wherein the treatment further comprises treatment with a second compound which is an acethylcholinesterase inhibitor.
   E39. A pharmaceutical composition comprising a compound according to any of embodiments 1-31, and one or more pharmaceutically acceptable carrier or excipient.
   E40. The composition according to embodiment 39, which composition additionally comprises a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E41. The composition according to embodiment 40, wherein said second compound is an acetylcholinesterase inhibitor.
   E42. A kit comprising a compound according to any of embodiments 1-31, together with a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E43. The kit according to embodiment 42, wherein said second compound is an acetylcholinesterase inhibitor.
   E44. A method for the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain, which method comprises the administration of a therapeutically effective amount of a compound according to any of embodiments 1-31 to a patient in need thereof.
   E45. The method according to embodiment 44, wherein said disease or disorder is selected from schizophrenia; AD; ADHD; autism spectrum disorders; PD; amyotrophic lateral sclerosis; Huntington's disease; dementia associated with Lewy bodies and pain.
   E46. The method according to embodiment 45, wherein said disease or disorder is selected from schizophrenia; AD; ADHD and autism spectrum disorders.
   E47. The method according to embodiment 46, wherein said treatment comprises the treatment of negative and/or cognitive symptoms of schizophrenia.
   E48. Use of a compound according to any of embodiments 1-31, for the manufacture of a medicament for the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); traumatic brain injury: epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.
   E49. The use according to embodiment 47, wherein said disease or disorder is selected from schizophrenia; AD; ADHD; autism spectrum disorders; PO; amyotrophic lateral sclerosis; Huntington's disease; dementia associated with Lewy bodies and pain.
   E50. The use according to embodiment 49, wherein said disease or disorder is selected from schizophrenia; AD; ADHD and autism spectrum disorders.
   E51. The use according to embodiment 50, wherein said disease is the positive, negative and/or cognitive symptoms of schizophrenia.
   E52. The use according to any of embodiments 48-51, wherein said manufacture further comprises the use of a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E53. The use according to embodiment 52, wherein said second compound is an acetylcholinesterase inhibitor.

The compounds of the invention may exist in unsolvated as well as in solvated forms in which the solvent molecules are selected from pharmaceutically acceptable solvents such as water, ethanol and the like. In general, such solvated forms are considered equivalent to the unsolvated forms for the purposes of this invention.

Included also in this invention are isotopically labeled compounds, which are identical to those claimed in formula [I], wherein one or more atoms are represented by an atom of the same element having an atomic mass or mass number different from the atomic mass or mass number usually found in nature (e.g., ²H, ³H, ¹¹C, ¹³C, ¹⁵N, ¹⁸F and the like).

The compounds of the present invention may have one or more asymmetric centres and it is intended that any optical isomers (i.e. enantiomers or diastereomers), in the form of separated, pure or partially purified optical isomers and any mixtures thereof including racemic mixtures, i.e. a mixture of stereoisomers, are included within the scope of the invention.

When R7 is not H, the compounds of the present invention may have an asymmetric centre at C-R7 indicated with an arrow below. In a preferred embodiment, the compounds of the invention are manufactured from a chiral intermediate e.g. (+)-1-(2-Methyl-1H-indol-5-yl)ethylamine (IM9) with the stereochemistry around R7 as indicated by the arrow below.

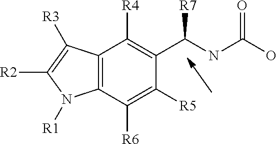

In this context is understood that when specifying the enantiomeric form, then the compound is in enantiomeric excess, e.g. essentially in a pure, mono-enantiomeric form. Accordingly, one embodiment of the invention relates to a compound of the invention having an enantiomeric excess of at least 60%, at least 70%, at least 80%, at least 85%, at least 90%, at least 96%, preferably at least 98%.

Racemic forms can be resolved into the optical antipodes by known methods, for example by separation of diastereomeric salts thereof with an optically active acid, and liberating the optically active amine compound by treatment with a base. Another method for resolving racemates into the optical antipodes is based upon chromatography of an optically active matrix. The compounds of the present invention may also be resolved by the formation of diastereomeric derivatives. Additional methods for the resolution of optical isomers, known to those skilled in the art, may be used. Such methods include those discussed by J. Jaques, A. Collet and S. Wilen in “Enantiomers, Racemates, and Resolutions”. John Wiley and Sons, New York (1981). Optically active compounds can also be prepared from optically active starting materials.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms that the compounds are able to form are included within the scope of the present invention.

The inventors have found that a broad selection of the compounds of the invention-possesses improved aqueous solubility compared to prior art compounds. In particular, the choice of the substituents R1-R5 has an impact on the solubility of the compounds. Especially compounds of the invention wherein R2 is not trifluoromethyl have shown to possess improved aqueous solubility compared to prior art compounds which are PAMs of the NNRs.

The compounds of the present invention may be administered alone as a pure compound or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Other suitable administration forms include suppositories, sprays, ointments, cremes, gels, inhalants, dermal patches, implants, etc.

In one embodiment, the compound of the present invention is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 1-1000 mg/day of a compound of the present invention, such as 1-500 mg/day, such as 1-100 mg/day or 1-50 mg/day. Conveniently, the compounds of the invention are administered in a unit dosage form containing said compounds in an amount of about 0.1 to 500 mg, such as 10 mg, 50 mg 100 mg, 150 mg, 200 mg or 250 mg of a compound of the present invention.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutical acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the various aspects, embodiments, implementations and features of the invention mentioned herein may be claimed separately, or in any combination.

### EXAMPLES

The invention will be illustrated by the following non-limiting examples.

### Synthetic Routes

### Methods

NMR spectra were recorded on a Bruker 600-Avance-III spectrometer equipped with a 5 mm TCI cryoprobe operating at 600.16 MHz for ¹H and 150.91 MHz for ¹³C or a Bruker 500-Avance or a Bruker DRx-500 spectrometer equipped with a 5 mm QNP probe operating at 500.13 MHz for ¹H and 125.76 MHz for ¹³C or a Varian 400MR instrument at T=298.15 K or at 400 MHz or a Varian vnmrs instrument. TMS was used as internal reference for ¹H and the solvent was used as internal reference for ¹³C. DMSO-d₆ or CDCl₃ was used as solvent.

### LCMS Methods

### Method A:

LC-MS were run on a Sciex API150EX equipped with APPI-source operating in positive ion mode. The HPLC consisted of Shimadzu LC10-ADvp LC pumps, SPD-M20A PDA detector (operating at 254 nM) and SCL-10A system controller. Autosampler was Gilson 215, Colomn oven was a Jones Chromatography 7990R and ELS detector was a Sedere Sedex 85. LC-conditions: The column was a Waters Symmetry C-18. 4.6×30 mm, 3.5 μm operating at 60° C. with 3.0 ml/min of a binary gradient consisting of water+0.05 TFA (A) and methanol+0.05% TFA (B).

### Gradient:

| | |
|---|---|
| 0.01 min | 17% B |
| 0.27 min | 28% B |
| 0.53 min | 39% B |
| 0.80 min | 50% B |
| 1.07 min | 59% B |
| 1.34 min | 68% B |
| 1.60 min | 78% B |
| 1.87 min | 86% B |
| 2.14 min | 93% B |
| 2.38 min | 100% B |
| 2.40 min | 17% B |
| 2.80 min | 17% B |

Total run time: 2.8 min

### Method B:

LC-MS were run on Waters Aquity HPLC-MS consisting of Waters Aquity including column manager, binary solvent manager, sample organizer, PDA detector (operating at 254 nM), ELS detector and SQ-MS equipped with APPI-source operating in positive ion mode (ESI-source, APCI-source positive ion mode, negative ion mode).

LC-conditions: The column was a Acquity HPLC BEH C18 1.7 μm; 2.1×50 mm operating at 60° C. with 1.2 ml/min of a binary gradient consisting of water+0.01% formic acid (A) and acetonitrile +5% water+0.1% formic acid (8).

### Gradient:

| Time, min. | % B |
|---|---|
| 0.00 | 10.0 |
| 1.00 | 99.9 |
| 1.01 | 10.0 |
| 1.15 | 10.0 |

### Method C:

As method B but using the following LC-conditions:

The column was a Acquity HPLC BEH C18 1.7 μm; 2.1×50 mm operating at 60° C. with 1.2 ml/min of a binary gradient consisting of water+0.05% TFA (A) and acetonitrile+5% water+0.035% TFA (B).

### Gradient:

| Time, min. | % B |
|---|---|
| 0.00 | 10.0 |
| 1.00 | 100 |
| 1.01 | 10.0 |
| 1.15 | 10.0 |

### Method D:

As method D but using:

### Gradient:

| Time, min. | % B |
|---|---|
| 0.00 | 2 |
| 1.00 | 100 |
| 1.01 | 2 |
| 1.15 | 2 |

### Preparative Methods

### Method E:

Preparative supercritical fluid chromatography (SFC) was performed on a Berger Multigram II operating at 50 ml/min at 35° C. and 100 bar backpressure using stacked injections. The column was a Phenomenex Lux 5 μm Cellulose-1 (250′21.2 mm). The eluent was CO₂ (70%) and methanol+0.5% diethylamine (30%).

### Method F:

Preparative supercritical fluid chromatography (SFC) was performed on a Thar SFC-80 system operating at 80 g/min and 80 bar backpressure. The column was a Chiralcel OJ-H (250×30)mm, 5 μm. Co Solvent: 20% of 0.5% Isopropyl amine in Isopropanol; Diluent: Methanol+Isopropanol (50+50).

### Method G:

Preparative HPLC was performed on a Shimadzu LC-8A instrument fitted with a Phenomenex Gemini C18 25021.2 mm*5 μm column using water and acetonitrile as the eluents. Mobile phase A: water (containing 0.05% ammonia, v/v), mobile phase B: acetonitrile. Gradient: B from 25% to 55% in 25 min.

### Method H:

Preparative HPLC was performed on a Shimadzu FRC-10A instrument fitted with a Synergi C18 column (250 mm*50 mm, 10 μm), using water and acetonitrile as the eluents. Mobile phase A: water (containing 0.1% TFA, v/v), mobile phase B: acetonitrile. Gradient: 10-40% B, 0-22 min; 40-70% B, 23-35 min.

### Method I:

Preparative HPLC was performed on a Gilson-GX-821 Autosampler (Pumps-333 &334 Detector-UV) instrument fitted with a Chiralpak-IA column (250*30*5 μm) using N-Hexane and Ethanol as the eluents. Mobile phase A: N-Hexane (85%), mobile phase B: Ethanol (15%). Method: Isocratic with runtime 30 min.

### Method J:

Preparative LCMS was performed on a system consisting of Sciex Applied Biosystems API150EX single qaudrupole mass spectrometer and atmospheric pressure photo ionisation (APPI) ion source, Gilson 333/334 pumps, Gilson UV/VIS155 detector, and Gilson GX 281 sampler/fraction collector.

Duration: app. 7.5 min. pr sample injection. Column: Sunfire Prep C18 5 μm, 19×50 mm, Nebulizer temp: 425° C., Injection volume: 0-200 μL, Flow: 40 ml/min, Temperature: 40° C. Solvents: A: Water containing 0.05% v/v TFA, B: Methanol containing 0.05% v/v TFA.

### Gradient:

| Time, min. | % B |
|---|---|
| 0.00 | 10.0 |
| 3.00 | 100.0 |
| 3.20 | 100.0 |
| 3.21 | 10.0 |

Injection into mobile phase B—time from injection to application on column/mixture with mobile phase A: 0.7 minutes (prior to T=0 minute in gradient program)

### Method K:

Preparative supercritical fluid chromatography (SFC) was performed on a Berger MultiGram system operating at 65 mL/min and 100 bar backpressure. The column was a DAICEL AS250 mm*30 mm, 5 μm. Mobile phase: A: Supercritical CO₂, B: EtOH(0.05% NH₃H₂O), A:B=75:25. Column Temp 38° C.

### Preparation of Intermediates

### Preparation of Indoles

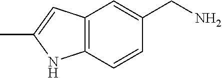

### IM1: C-(2-Methyl-1H-indol-5-yl-methylamine

### Step 1:

5-bromo-2-methylindole (6.00 g, 28.6 mmol) was dissolved in quinoline (50 mL). Copper cyanide (7.46 g, 83.3 mmol) was added. The mixture was refluxed for 1 hour.

The mixture was cooled to room temperature, diluted with EtOAc (500 mL) and washed with ice-cold hydrochloric acid (1M). The organic layer was washed with brine, dried over MgSO₄, filtered and evaporated to dryness. Flash chromatography (silica, heptanes:EtOAc 1:1) gave 2-methyl-1H-indole-5-carbonitrile as a brown solid (4.11 g, 83%).

### Step 2:

LiAlH₄ (9.70 g, 255 mmol) was suspended in THF (400 mL) and the resulting suspension was cooled in a ice/water bath. A solution of 2-methyl-1H-indole-5-carbonitrile (11.4 g, 73 mmol) in THF (100 mL) was added dropwise over 30 minutes while keeping the internal temperature at 5-7° C. The mixture was then heated to reflux for 1 h. The mixture was cooled in an ice-water bath. To this mixture was sequentially added 20 ml water, 10 ml sodium hydroxide-solution (5M) and 50 ml water and the mixture was stirred for 10 minutes. A generous amount of MgSO₄ was added. The mixture was stirred for additional 10 minutes and then filtered. The residue was thoroughly extracted with THF. To the combined filtrates was added EtOAc (1 L) and this solution was dried with MgSO₄. The mixture was filtered and evaporated to dryness to give the title compound as a light-yellow powder (11.9 g, 97%).

1H NMR (500 MHz, DMSO) δ 10.76 (s, 1H), 7.31 (s, 1H), 7.18 (d, J=8.2 Hz, 1H), 6.95 (d, J=8.2 Hz, 1H), 6.05 (s, 1H), 3.73 (s, 2H), 2.36 (s, 3H).

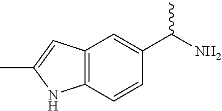

### IM2: rac-1-(2-Methyl-1H-indol-5-yl)-ethylamine

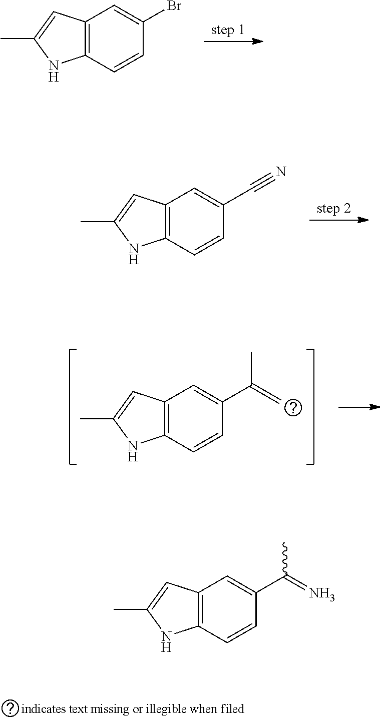

### Step 1: 2-Methyl-1H-indole-5-carbonitrile

A round bottomed flask was charged with 5-bromo-2-methylindole (20.0 g, 95.2 mmol) in quinoline (200 mL). Copper cyanide (13.30 g, 148.5 mmol) was added. The mixture was refluxed for 1 hour. Formation of a dense precipitate impeded the stirring early in the reaction, later on the precipitate disappeared. The mixture was cooled to room temperature and poured into EtOAc (0.5 L) and 6N HCl (250 mL). The mixture was filtered. The filtrate was thoroughly washed with ice-cold hydrochloric acid (2M). The organic layer was then washed with brine, dried over MgSO₄ and evaporated to dryness. Flash chromatography (silica, EtOAc:heptane 1:1) gave 2-methyl-1H-indole-5-carbonitrile (13.0 g, 80%) as a light-brown powder, Mp 125-7

### Step 2:

A dry round-bottomed flask was charged with 2-methyl-1H-indole-5-carbonitrile (2.00 g, 12.8 mmol) in THF (20 mL). A solution of methylmagnesiumbromide in toluene (1.40 M, 45.7 mL) was added dropwise. The mixture was heated to reflux overnight. The mixture was cooled to room temperature. Methanol (3 mL) was added and the mixture was stirred for 1 h. The resulting suspension was filtered and the filtrate was evaporated to dryness. The residue was suspended in methanol (30 mL) and was cooled to 0-5° C. Sodium tetrahydroborate (0.969 g, 25.6 mmol) was added portion wise over 20 minutes and then the mixture was left with stirring overnight at room temperature followed by reflux for 2 hours. The mixture was cooled to 10° C. and a mixture of water (8 mL) and acetic acid (3 mL) was added. The reaction was stirred for 30 minutes at room temperature, heated to reflux for 1 hour and then evaporated to dryness. The residue was diluted with ice water. NaOH (conc.) was added until pH reached 8-10 and the mixture was extracted with EtOAc (3×100 mL). The combined organic layers were washed with brine (100 mL), dried over MgSO₄ and evaporated to dryness. Flash chromatography (silica, EtOAc:ethanol:TEA 100:5:4) gave racemic 1-(2-methyl-1H-indol-5-yl)ethylamine as an oil (1.62 g, 73%).

1H NMR (500 MHz, CDCl₃) 6^{,} 8.35 (br s, 1H), 7.51 (s, 1H), 7.27-7.22 (m, 1H), 7.16-7.10 (m, 1H), 6.22 (s, 1H), 4.27-4.21 (m, 1H), 2.44 (s, 3H), 1.60 (br s, 2H), 1.50 (d, J=6.9 Hz, 3H).

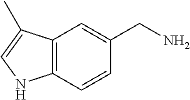

### IM3: C-(3-Methyl-1H-indol-5-yl)-methylamine

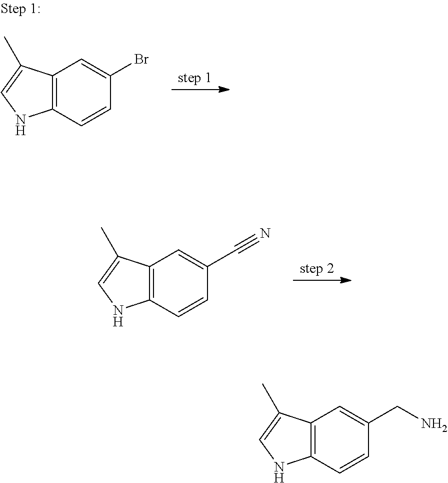

A dry round bottomed flask was charged with 5-bromo-3-methylindole (5.00 g, 23.8 mmol) in quinoline (50 mL). Copper cyanide (6.40 g, 71.4 mmol) was added and the mixture was refluxed for 1 hour with rigorous stirring. The mixture was allowed to reach room temperature and then poured into EtOAc (400 mL) and stirred for 5 minutes. The suspension was filtered. The filtrate was washed with ice-cold hydrochloric acid (2M) followed by brine. The organic layer was dried over MgSO₄, filtered and evaporated to dryness. Flash chromatography (silica, EtOAc:heptanes 1:1) gave 3-methyl-1H-indole-5-carbonitrile as a dark-brown powder (2.90 g. 78%).

1H NMR (500 MHz, DMSO) δ 11.34 (s, 1H), 8.03 (sf 1H), 7.52-7.47 (m, 1H), 7.43-7.38 (m, 1H), 7.32 (s, 1H), 2.29 (s, 3H).

### Step-2:

A dry round-bottomed flask was charged with LiAlH₄ (2.47 g, 65.0 mmol) in THF (75 mL). The mixture was cooled to 0° C. A solution of 3-methyl-1H-indole-5-carbonitrile (2.90 g, 18.6 mmol) in THF (50.0 mL) was added dropwise over 10 minutes keeping the internal temperature at 8-12° C. The mixture was subsequently refluxed for 45 minutes. The mixture was cooled to 0° C. and carefully quenched with water (5 mL) followed by sodium hydroxide-solution (5M, 2.5 mL) and finally water (12.5 ml). The mixture was stirred for 5 minutes. A generous amount of MgSO₄ was added. The mixture was stirred for 10 minutes, filtered and evaporated to dryness. Flash chromatography (silica, EtOAc:heptanes 1:1 to EtOAc:ethanol:triethylamine 70:25:5) gave the title compound IM3 as a light-brownish powder (1.75 g, 59%).

1H NMR (500 MHz, DMSO) δ 10.62 (s, 1H), 7.41 (s, 1H), 7.29-7.22 (m, 1H), 7.08-7.02 (m, 2H), 3.79 (s, 2H), 2.28-2.21 (m, 3H).

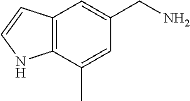

### IM4: C-(7-Methyl-1H-indol-5-yl)-methylamine

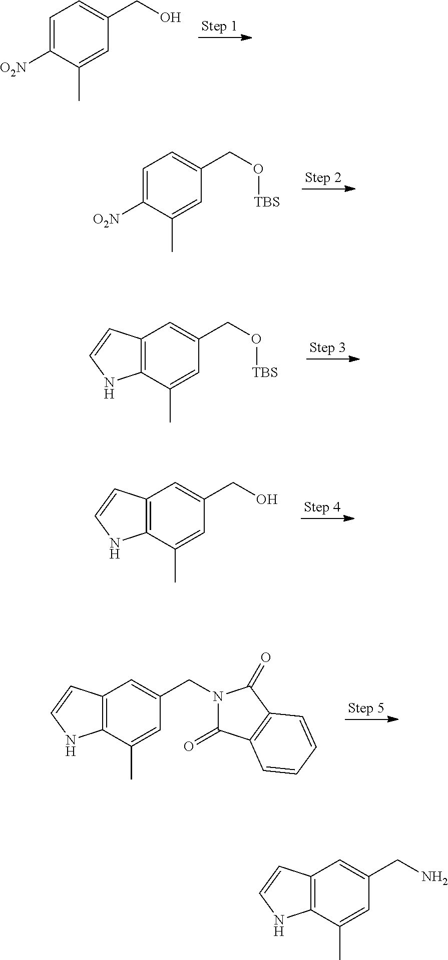

### Step 1:

A round-bottomed flask was charged with 3-methyl-4-nitrobenzyl alcohol (30.0 g, 179 mmol) in methylene chloride (200 mL). To this solution was added triethylamine (40.0 mL, 287 mmol) and 4-dimethylaminopyridine (2.20 g, 18.0 mmol). The solution was cooled to 0° C. A solution of tert-butyldimethylsityl chloride (29.8 g, 197 mmol) dissolved in methylene chloride (50 mL) was added dropwise over 10 minutes keeping the internal temperature at 4-11° C. The mixture was then stirred at room temperature over night. The mixture was filtered and evaporated to dryness. Flash chromatography (silica, heptane:EtOAc 4:1) gave tert-butyldimethyl-(3-methyl-4-nitro-benzyloxy)-silane as a yellow oil (53.1 g, quant.)

### Step 2:

A round-bottomed flask was charged with tert-butyl-dimethyl-(3-methyl-4-nitro-benzyloxy)silane (42.0 g, 142 mmol) in THF (800 mL). The mixture was cooled in a dry ice/acetone bath to −40° C. A solution of vinylmagnesium bromide in THF (1.0 M, 440.0 mL) was added over 10 minutes keeping the internal temperature between −40 and −25° C. The mixture was then stirred for 20 minutes at −35° C. The cold mixture was poured into aqueous ammonium chloride-solution (10%, 1.5 L). This mixture was extracted with EtOAc. The combined organic layers were washed with brine, dried over MgSO₄, filtered and evaporated to dryness. Flash chromatography (silica, heptane:EtOAc 4:1) gave 5-(tert-butyl-dimethyl-silanyloxymethyl)-7-methyl-1H-indole as a red oil (8.95 g, 23%).

1H NMR (500 MHz, DMSO) δ 10.99 (s, 1H), 7.34-7.26 (m, 2H), 6.83 (s, 1H), 6.42-6.38 (m, 1H), 4.72 (s, 2H), 2.46 (s, 3H), 0.94-0.87 (m, 9H), 0.08 (s, 6H).

### Step-3:

A round-bottomed flask was charged with 5-(tert-butyl-dimethyl-silanyloxymethyl)-7-methyl-1H-indole (8.95 g, 32.5 mmol) in THF (100 mL) and cooled in an ice-water batch. A solution of tetra-n-butylammonium fluoride in THF (1.0M, 34.0 mL) was added dropwise over 10 minutes at 2-4° C. The mixture was stirred at room temperature for 3 hours. The mixture was poured into brine and extracted with EtOAc. The combined organic layers were washed with brine, dried over MgSO₄, filtered and evaporated to dryness. Flash chromatography (silica, EtOAc:heptanes 1:1) gave crude (7-methyl-1H-indol-5-yl)-methanol as a dark-red oil (3.44 g, 55%)_{.}

### Step-4:

A round-bottomed flask was charged with (7-methyl-1H-indol-5-yl)-methanol (3.44 g, 18.1 mmol) in THF (125 mL). To this solution was added phthalimide (3.34 g, 22.7 mmol) and triphenylphosphine (7.61 g, 29.0 mmol. The mixture was stirred for 10 minutes and then cooled in an ice-water bath. A solution of diethyl azodicarboxylate in toluene (0.225M, 141 mL) was added dropwise over 10 minutes keeping the internal temperature at 10-25° C. The mixture was then stirred for 2 hours at room temperature. The mixture was concentrated to a volume of approx. 150 ml. Flash chromatography of this material (silica, EtOAc:heptanes 1:1) gave crude 2-(7-methyl-1H-indol-5-ylmethyl)-isoindole-1,3-dione (3.27 g, 47%) as a pale-yellow powder.

### Step-5:

A round-bottomed flask was charged with 247-methyl-1H-indol-5-ylmethyl)isoindole-1,3-dione (3.27 g, 8.45 mmol) in a mixture of THF (30 mL) and methanol (100 mL). Hydrazine hydrate (1.23 mL, 25.3 mmol) was added. The mixture was refluxed for 3 hours. The mixture was then concentrated to a volume of approx. 50 mL. A copious white precipitate formed. The mixture was filtered and the filtrate was subjected to flash chromatography (silica, EtOAc:ethanol:triethylamine 85:10:5) to give compound IM4 as a tan solid (0.771 g, 54%).

1H NMR (600 MHz, DMSO) δ 10.98 (s, 1H), 7.32-7.23 (m, 2H), 6.87 (s, 1H), 6.38-6.31 (m, 1H), 3.76 (s, 2H), 2.44 (s, 3H).

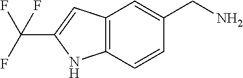

### IM5: C-(2-Trifluoromethyl-1H-indol-5-yl)-methylamine

The compound was prepared as described in WO2009/127678A1.

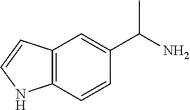

### IM6: 1-(1H-Indol-5-yl)-ethylamine

The compound was purchased from Chembridge, Catalog No 4102139.

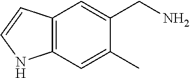

### IM7: C-(6-Methyl-1H-indol-5-yl)-methylamine

A dry round-bottomed flask was charged with LiAlH₄(1.09 g, 28.8 mmol) suspended in 60 ml THF. The suspension was cooled to 0-5° C. A solution of 5-cyano-6-methyl indole (1.50 g. 9.60 mmol) in THF (30 mL) was added dropwise. After addition was complete the reaction was heated to reflux for 3 hours. The reaction was quenched by the careful addition of water (0.5 mL) then 2 N NaOH (0.5 mL) and finally water (2.5 mL). To the reaction mixture was added MgSO₄ and the mixture was stirred for 10 minutes and was then filtered. The filtrate was evaporated to dryness. Flash chromatography (silica, EtOAc:ethanol:TEA 90:10:4) gave the title compound as a solid (1.35 g, 88%).

1H NMR (500 MHz, DMSO) δ 10.82 (s, 1H), 7.46 (s, 1H), 7.22-7.18 (m, 1H), 7.15 (s, 1H), 6.32 (s, 1H), 3.75 (s, 2H), 2.37 (s, 3H).

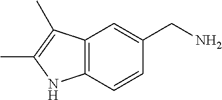

### IM8: C-(2,3-Dimethyl-1H-indol-5-yl)-methylamine

The compound was purchased from Enamine, Catalog No. EN300-55879.

### IM9: (+)-R-1-(2-Methyl-1H-indol-5-yl)-ethylamine and IM13 (−)-S-1-(2-Methyl-1H-indol-5-yl)ethylamine

Approximately 1.5 g of racemic material IM2 was subjected to preparative SFC separation of isomers (Method E) to give:

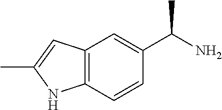

### IM9: (+)-R-1-(2-Methyl-1H-indol-5-yl)-ethylamine

0.170 g isolated (Method E). Optical rotation +24.7° (0.5% in CH₃OH). A crystal of a salt of amine IM9 with N-Ac-L-phenyl-alanine (S)-configuration) shows that the absolute configuration of IM9 is (R).

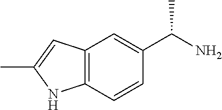

### IM13: (−)-S-1-(2-Methyl-1H-indol-5-yl)-ethylamine

0.278 g isolated (Method E).

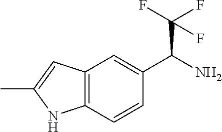

### IM10: (−)-(S)-2,2,2-Trifluoro-1-(2-methyl-1H-indol-5-34)-ethylamine

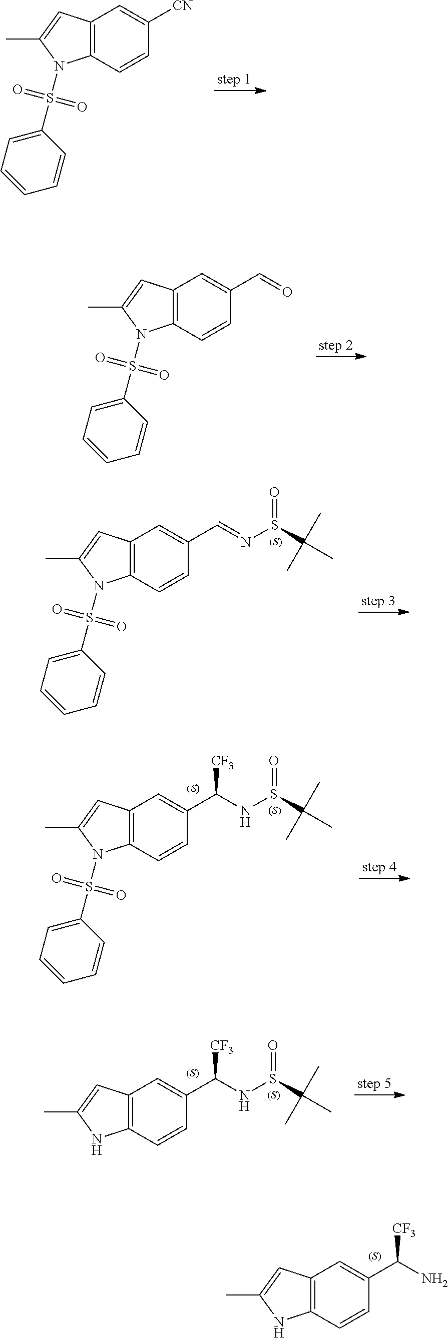

### Step 1:

To a solution of 1-benzenesulfonyl-2-methyl-1H-indole-5-carbonitrile (10.0 g, 33.8 mmol) in methylene chloride (100 mL) was added DIBAL-H (1M in toluene, 50.6 mL, 50.7 mmol) dropwise at −78° C. The reaction mixture was stirred at −78° C. for 2 h and was then carefully diluted with 2N HCl (200 mL). The mixture was extracted with EtOAc (2×250 mL). The combined organic layers were dried over Na₂SO₄ and evaporated to dryness. The crude material was washed with diethyl ether to give 1-benzenesulfonyl-2-methyl-1H-indole-5-carbaldehyde as a yellow solid (9.0 g, 89%). The material was used in the next step without further purification.

### Step 2:

To a solution of 1-benzenesulfonyl-2-methyl-1H-indole-5-carbaldehyde (25.0 g, 83.6 mmol) in anhydrous THF (500 mL) was added (S)-2-methyl-2-propanesulfinamide (15.2 g, 125.4 mmol) and Ti(OEt)₄ (38.1 g, 167.2 mmol) at room temperature. The reaction mixture was stirred at reflux for 6 h. The reaction mixture was then diluted with brine (500 mL) and the resulting mixture was extracted with EtOAc (2×500 mL). The combined organic layers were dried over anhydrous Na₂SO₄ and evaporated to dryness to give (S)-2-methyl-propane-2-sulfinic acid 1-(1-benzenesulfonyl-2-methyl-1H-indol-5-yl)-methylideneamide as a pale yellow liquid (32.0 g, 95%) which was used in the next step without further purification.

### Step 3:

This step was carried out as described in Olah, G. et al, Angew. Chem. Int. Ed. 2001, 40, 3, 589-590: To a solution of (S)-2-methyl-propane-2-sulfinic acid 1-(1-benzenesulfonyl-2-methyl-1H-indol-5-yl)-methylideneamide (2.0 g, 4.97 mmol) in dry THF (60 mL), was added tetrabutylammonium difluorotriphenylsilicate (5.3 g, 9.95 mmol) and TMSCF₃ (1.41 g, 9.95 mmol) at −55° C. The reaction mixture was stirred at −55° C.-0° C. for 1 h. The reaction mixture was then diluted with saturated aqueous NH₄Cl solution (50 mL). The mixture was extracted with EtOAc (2×50 mL). The combined organic layers were dried over Na₂SO₄ and evaporated to dryness. Flash chromatography (silica, EtOAc:petroleum ether 1:3) gave (S)-2-methylpropane-2-sulfinic acid [(S)-1-(1-benzenesulfonyl-2-methyl-1H-indol-5-yl)-2,2,2-trifluoro-ethyl]-amide as an off-white solid (1.8 g, 77%).

### Step 4:

To a solution of (S)-2-methyl-propane-2-sulfinic acid [(S)-1-(1-benzenesulfonyl-2-methyl-1H-indol-5-yl)-2,2,2-trifluoro-ethyl]-amide (8.0 g, 16.94 mmol) in MeOH (160 mL) was added KOH (14.25 g, 254 mmol) at 0° C. The reaction mixture was stirred at 25° C. for 48 h. The reaction mixture was poured into water and stirred for 30 min. The mixture was filtered and the remanence washed with n-pentane (50 mL) to give (S)-2-methyl-propane-2-sulfinic acid [(S)-2,2,2-trifluoro-1-(2-methyl-1H-indol-5-yl)-ethyl]-amide as an off-white solid (3.3 g, 59%).

### Step 5:

To a solution of (S)-2-methyl-propane-2-sulfinic acid [(S)-2,2,2-trifluoro-1-(2-methyl-1H-indol-5-yl)-ethyl]-amide (6.3 g, 19.0 mmol) in diethyl ether (64 mL) at 0° C. was added 2M HCl in diethyl ether (32 mL). The reaction mixture was subsequently stirred at 25° C. for 4 h. The reaction mixture was then filtered and the remanence washed with EtOAc and dried under vacuum. The crude salt was dissolved in water (120 mL) and basified with 1M NaOH. The resulting mixture was filtered and the remanence was washed with water (60 mL) to give the title compound IM10 as a pale brown powder (3.5 g, 81%).

1H NMR (DMSO, 400 MHz) δ 10.91 (1H, s), 7.48 (1H, s), 7.23-7.21 (1H, d), 7.08-7.06 (1H, d), 6.10 (1H, s), 4.44-4.43 (1H, m), 2.37 (3H, br s).

(Specific optical rotation −20.1°, 0.51% MeOH)

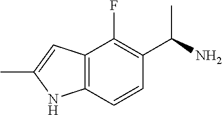

### IM11: (R)-1-(4-Fluoro-2-methyl-1H-indol-5-yl)-ethylamine (major) and (S)-1-(4-Fluoro-2-methyl-1H-indol-5-yl)-ethylamine (minor)

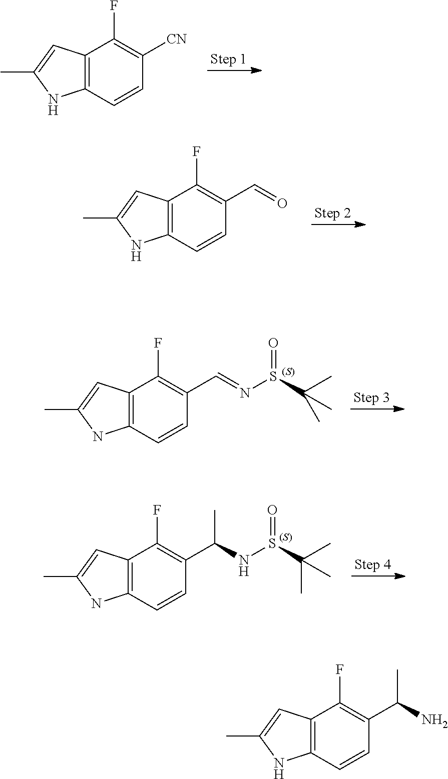

### Step 1:

DIBAL-H (0.45 mL, 0.5 mmol, 1.0 M in THF) was added to a mixture of 4-fluoro-2-methyl-1H-indole-5-carbonitrile (50 mg, 0.29 mmol) in dry THF (0.5 mL) at 0° C. The reaction mixture was stirred at 0° C. for 30 min and then stirred without cooling overnight. The mixture was quenched with water and extracted with EtOAc (200 mL). The organic layer was dried over Na₂SO₄ and evaporated to dryness. The residue was purified by pTLC to give 4-fluoro-2-methyl-1H-indole-5-carbaldehyde as a white solid (23 mg, 45%).

1HNMR (CDCl₃, 400 MHz) δ 10.35 (s, 1H), 8.25 (s, 1H), 7.53 (m, 1H), 7.05 (m, 1H), 6.37 (m, 1H), 2.40 (s, 3H).

### Step 2:

A mixture of 4-fluoro-2-methyl-1H-indole-5-carbaldehyde (1.5 g, 8.5 mmol) in THF (30 mL) was stirred at room temperature. To this mixture was added (S)-2-methyl-2-propanesulfinamide (2.0 g, 17.0 mmol) and Ti(OEt)₄ (5.8 g, 25.5 mmol) and the mixture was refluxed overnight. The mixture was then cooled, diluted with water and extracted with EtOAc (1000 mL). The organic layer was dried over Na₂SO₄ and evaporated to dryness to give crude (S)-2-methyl-propane-2-sulfinic acid 1-(4-fluoro-2-methyl-1H-indol-5-yl)methylideneamide as a yellow oil (2.0 g, 87%) which was used without further purification.

### Step 3:

CH₃MgBr (7.3 mL, 22.0 mmol, 3.0 M in THF) was added dropwise to a solution of (S)-2-methyl-propane-2-sulfinic acid 1-(4-fluoro-2-methyl-1H-indol-5-yl)-methylideneamide (2.0 g, 8.8 mmol) in dry THF (35 mL) at −78° C. Cooling was then removed and the reaction mixture was stirred at room temperature for 1 h. Another 7.3 mL of CH₃MgBr (3.0 M in THF) was added and the mixture was stirred at room temperature overnight. The mixture was quenched with water and extracted with EtOAc (2000 mL). The organic layer was washed with water, brine and dried over Na₂SO₄ and evaporated to dryness. Flash chromatography (silica, ElOAc:petroleum ether 1:2010 1:2) gave (S)-2-methyl-propane-2-sulfinic acid [(R)-1-(4-fluoro-2-methyl-1H-indol-5-yl)-ethyl]-amide as a yellow solid (1.2 g, 46%). The diastereomeric ratio was approx 7:3 by 1H NMR.

### Step 4:

(S)-2-methyl-propane-2-sulfinic acid [(R)-1-(4-fluoro-2-methyl-1H-indol-5-yl)-ethyl]-amide (1.2 g, 4.1 mmol), dr 7:3, was stirred at room temperature for 2 h in a solution of HCl in MeOH (20.0 mL, 4.0 M). The mixture was evaporated to dryness to give IM11. This mixture, which was a binary mixture of approx 7:3 R:S, was used without further purification and was used to make amides 53 and 54.

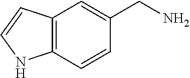

### IM12: C-(1H-Indol-5-yl)-methylamine

The compound was purchased from Sigma-Aldrich, Catalog No 655864.

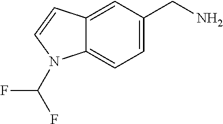

### IM14: C-(1-Difluoromethyl-1H-indol-5-yl)-methylamine

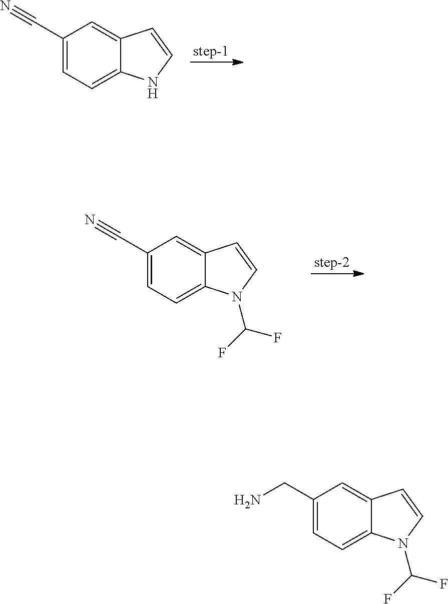

### Step 1:

To a stirred solution of 5-cyanoindole (10.0 g, 70 mmol) in methylene chloride (150 mL) was added a solution of NaOH (8.4 g, 210 mmol) in of water (12 mL). Benzyltriethyl ammonium chloride (0.795 g, 3.5 mmol) was added and the reaction mixture was stirred for 15 min. CHClF₂ gas was purged through the reaction mixture for 3 hours. The reaction mixture was diluted with methylene chloride and the organic layer was washed with water. The organic layer was dried over anhydrous Na₂SO₄ and evaporated to dryness. Flash chromatography (silica, EtOAc:hexanes 1:9) gave 1-difluoromethyl-1H-indole-5-carbonitrile as a colorless solid (9.1 g, 67%).

1H NMR (400 MHz, DMSO) δ 7.9 (1H, s), 7.6-7.7 (1H, m), 7.5-7.6 (1H, m), 7.1-7.4 (2H, m), 6.7 (1H, m).

### Step 2:

To a solution of 1-difluoromethyl-1H-indole-5-carbonitrile (9.0 g, 46.0 mmol) in methanol (40 mL) was carefully added Raney Ni (0.9 g) followed by ammonia in methanol (5M, 40 mL). The reaction mixture was hydrogenated at 60 psi for 24 hours. The reaction mixture was then filtered through a plug of celite and the filtrate was concentrated in vacuo. Flash chromatography (silica, MeOH:methylene chloride 15:85) gave the title compound IM14 as a brown semisolid (5.1 g, 56%).

1H NMR (400 MHz, DMSO) δ 7.8-8.1 (1H, m), 7.55-7.65 (3H, m), 7.32-7.26 (1H, m), 6.67 (1H, s), 3.8 (2H, s).

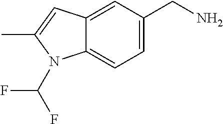

### IM15: C-(1-Difluoromethyl-2-methyl-1H-indol-5-yl)-methylamine

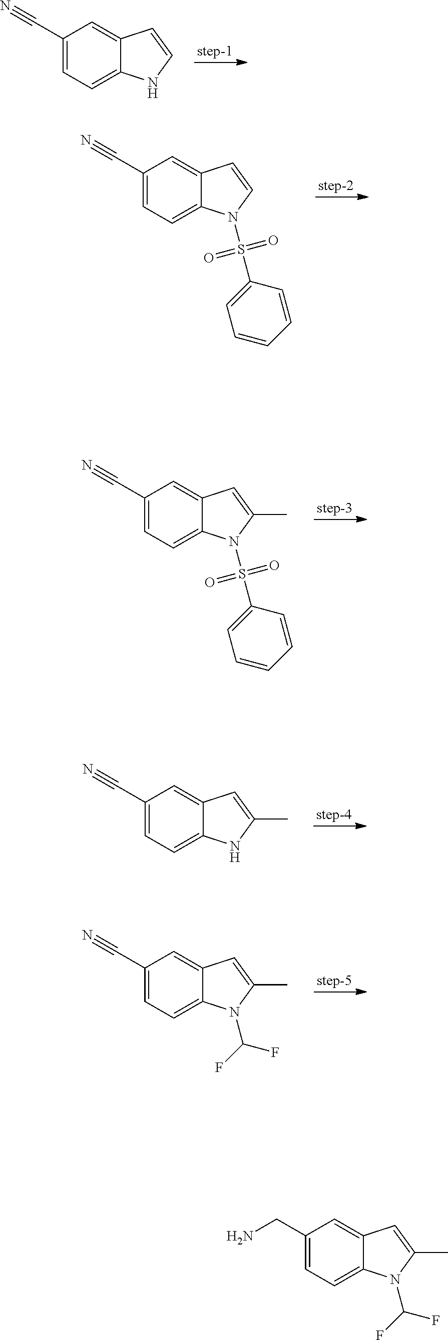

### Step 1:

To a suspension of sodium hydride (60%, 42.0 g, 1.053 mol) THF (600 mL) at 0° C. was slowly added a solution of 5-cyanoindole (50.0 g, 0.351 mol) in THF (200 mL). The mixture was stirred for 30 min. A solution of benzene sulfonyl chloride (111.5 g, 0.633 mol) in THF (200 mL) was then slowly added at 0° C. The mixture was allowed to reach room temperature and was then stirred for 15 h. EtOAc was added and the mixture was washed with 1N hydrochloric acid. The aqueous layer was extracted with EtOAc. The combined organic layers were dried over anhydrous Na₂SO, and concentrated to dryness. The resulting solid was washed with petroleum ether to give 1-benzenesulfonyl-1H-indole-5-carbonitrile as a yellow solid (80.0 g, 81%).

1H NMR (400 MHz, CDCl₃) δ 8.09-8.07 (1H, d); 7.90-7.88 (3H, m), 7.70-7.69 (1H, d), 7.61 (1H, s), 7.59-7.55 (1H, t), 7.50-7.47 (2H, t), 6.73-6.72 (1H, d).

### Step 2:

To a solution of LDA (1.8 M, 10 mL, 19.7 mmol) in THF (30 mL) at −78° C. under an atmosphere of nitrogen was added a solution of 1-benzenesulfonyl-1H-indole-5-carbonitrile (4.0 g, 14.1 mmol) in THF (15 mL). The mixture was stirred for 30 min, the temperature of the solution was slowly raised to −40° C. and after 30 min the mixture was re-cooled to −78° C. To this solution was added dropwise a solution of MeI (4.42 g, 31.1 mmol) in THF (15 mL). The reaction temperature was maintained at −78° C. for 2 h. The mixture was then allowed to reach room temperature and was left overnight. The mixture was diluted with EtOAc and the organic layer was washed with 1N hydrochloric acid. The organic layer was evaporated to dryness. Flash chromatography (silica, EtOAc:petroleum ether 1:4) gave 1-benzenesulfonyl-2-methyl-1H-indole-5-carbonitrile as an off-white solid (2.2 g, 52%).

1H NMR (400 MHz, DMSO) δ 8.22-8.19 (1H, m), 8.05 (1H, s) 7.94-7.92 (2H, m), 7.76-7.74 (1H, m), 7.70-7.68 (1H, m), 7.64-7.60 (2H, m), 6.71 (1H, s), 2.63 (3H, s).

### Step 3:

To a stirred solution of 1-benzenesulfonyl-2-methyl-1H-indole-5-carbonitrile in THF:MeOH (1:1, 36 mL) was added a solution of NaOH (3.9 g, 0.1 mol) in water (10 mL) dropwise at 0° C. The mixture was stirred for 30 min, cooling was removed and the mixture was left overnight. The mixture was concentrated in vacuo. The residue was diluted with water and the aqueous layer was extracted with EtOAc. The combined organic layers were dried over Na₂SO₄ and evaporated to dryness. Flash chromatography gave 2-methyl-1H-indole-5-carbonitrile as an off-white solid (0.85 g, 73%).

1H NMR (400 MHz, CDCl₃) δ 8.21 (1H, br s), 7.84 (1H, s), 7.36-7.25 (2H, m), 6.29 (1H, s), 2.47 (3H, s).

### Step 4:

To a stirred solution of 2-methyl-1H-indole-5-carbonitrile (8.0 g, 51.2 mmol) in methylene chloride (100 mL) at 0° C. was added NaOH (6.15 g, 154 mmol in 15 mL water) followed by benzyltriethyl ammonium chloride (0.58 g, 2.5 mmol). The mixture was stirred for 15 min. CHClF₂ gas was purged through the reaction mixture for 3 h. The reaction mixture was diluted with methylene chloride, dried over Na₂SO₄ and evaporated to dryness. Flash chromatography (silica, EtOAc:petroleum ether 1:4) gave 1-difluoromethyl-2-methyl-1H-indole-5-carbonitrile as an off-white solid (3.10 g, 29%).

1H NMR (400 MHz, CDCl₃) δ 7.83 (1H, s), 7.61-7.59 (1H, m), 7.47-7.45 (1H, m), 7.41-7.11 (1H, m), 6.39 (1H, s). 2.54 (3H, s).

### Step 5:

To a stirred solution of 1-difluoromethyl-2-methyl-1H-indole-5-carbonitrile (3.1 g, 15.0 mmol) in methanol (20 mL) was carefully added Raney Ni (0.35 g) followed by ammonia in methanol (20 mL, 5M). The mixture was hydrogenated at 60 psi for 12 h. The reaction mixture was then filtered through a plug of celite and the filtrate was evaporated to dryness. Flash chromatography (silica, MeOH:methylene chloride 1:9) gave the title compound IM15 as an off-white solid (2.6 g, 82%).

1H NMR (400 MHz, CD₃OD) δ 7.74 (1H, s), 7.59 (1H, s), 7.53-7.51 (1H, m), 7.16-7.14 (1H, m), 6.30 (1H, s), 3.84 (2H, br s), 2.48 (3H, s).

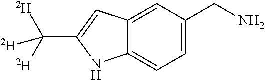

### IM16: C-(2-Methyl-d₃-1H-indol-5-yl)-methyl-amine

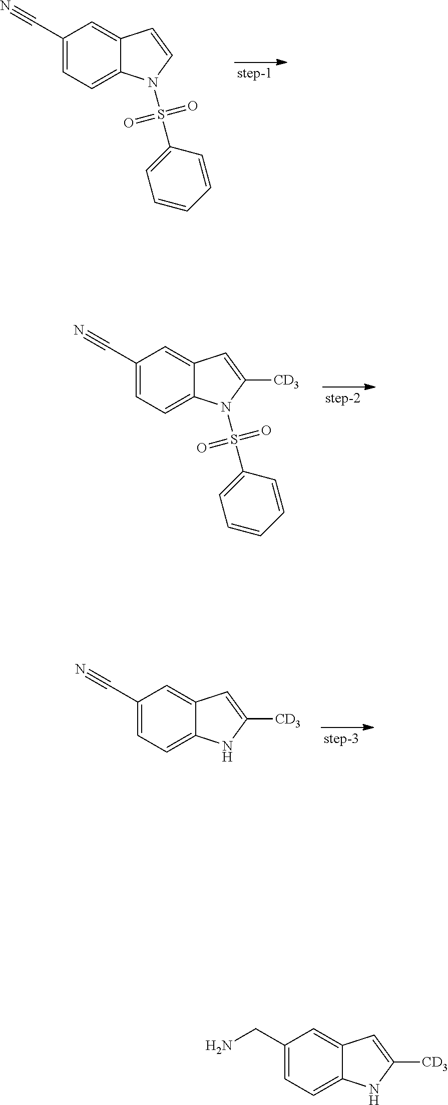

### Step 1:

LDA in THF (20.4 mL, 1.8 M solution, 36.87 mmol) was added to THF (20 mL) and the mixture was cooled at −78° C. A solution of 1-benzenesulfonyl-1H-indole-5-carbonitrile (8.0 g, 28.36 mmol) in THF (15 mL) was added dropwise. After 30 min the mixture was allowed to reach −40° C. After stirring at this temperature for 30 min the mixture was cooled to −78° C. Iodomethane-D₃ (8.22 g, 56.73 mmol) in THF (15 mL) was added dropwise. The mixture was allowed to reach room temperature overnight. EtOAc (60 mL) was added and the resulting mixture was washed with 1N hydrochloric acid (60 mL). The organic layer was evaporated to dryness. Flash chromatography (silica, EtOAc:petroleum ether 1:4) gave 1-benzenesulfonyl-2-methyl-d₃-1H-indole-5-carbonitrileas an off-white solid (3.2 g, 38%).

### Step 2:

To a vigorously stirred solution of 1-benzenesulfonyl-2-methyl-d₃-1H-indole-5-carbonitrile (2.2 g, 7.35 mmol) in THF: MeOH (18 mL:18 mL) was added 10 M NaOH (10 mL) dropwise at 0° C. The mixture was stirred for 30 min and then left without cooling overnight. The volatiles were removed under vacuum and the remanence was diluted with water (20 ml.) and extracted with ethyl acetate (2×40 mL). The combined organic layers were dried over Na₂SO₄ and evaporated to dryness. Flash chromatography (silica, EtOAc:petroleum ether 1:4) gave 2-methyl-d₃-1H-indole-5-carbonitrile as an off-white solid (800 mg, 68%).

### Step 3:

To a solution of 2-methyl-d₃-1H-indole-5-carbonitrile (600 mg, 3.77 mmol) in methanol (10 mL) was added Raney Ni (100 mg) followed by 5 M ammonia in methanol (15 mL). The reaction solution was hydrogenated at 60 psi for 12 h. The mixture was filtered through a plug of celite and the filtrate was evaporated to dryness. Flash chromatography (silica, MeOH:methylene chloride 12:88) gave the title compound IM16 as an off-white solid (0.55 g, 89%).

1H NMR (DMSO, 400 MHz) δ 10.74 (1H, s), 7.29 (1H, s), 7.17-7.15 (1H, d). 6.94-6.92 (1H, d), 6.03 (1H, s), 3.71 (2H, s), 1.66 (2H, broad).

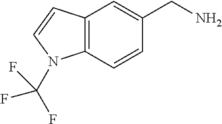

IM17: C-(1-Trifluoromethyl-1H-indol-5-yl)-methylamine

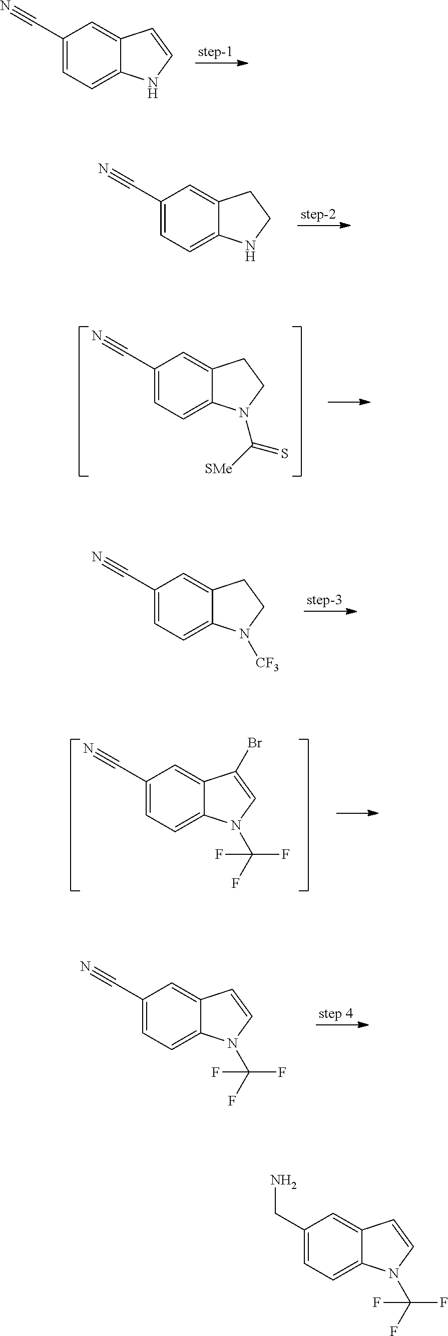

### Step 1:

To a stirred solution of 5-cyanoindole (20.0 g, 140 mmol) in trifluoroacetic acid (150 mL) at 0° C. was added triethylsilane (45.2 mL, 281 mmol). The reaction mixture was stirred for 4 h at 0° C. The reaction mixture was diluted with EtOAc (200 mL) and the organic layer was extracted with 1N HCl (200 mL). The aqueous layer was basified with 50% NaOH solution to pH 10-11 and extracted with EtOAc (2×200 mL). The combined organic layers were dried over anhydrous Na₂SO₄ and evaporated to dryness to give 2,3-dihydro-1H-indole-5-carbonitrile (6.2 g, 31%) as a pale yellow solid. The material was used without further purification.

### Step 2:

To a suspension of NaH (2.5 g, 108 mmol) in DMF (100 mL) at 0° C. was added 2,3-dihydro-1H-indole-5-carbonitrile (6.2 g, 43.0 mmol). After 15 min, CS₂ (3.8 mL, 64.6 mmol) was added followed by MeI (4.0 mL, 64.6 mmol). The reaction mixture was stirred for 1 h at room temperature. The reaction mixture was then was poured onto ice. The resulting mixture was filtered. The remanence was washed with methylene chloride to give 5-cyano-2,3-dihydroindole-1-carbodithioic acid methyl ester as a off-white solid (7.5 g, 76%). The material was used without further purification.

To a stirred solution of 5-cyano-2,3-dihydro-indole-1-carbodithioic acid methyl ester (8.0 g, 34.1 mmol) in methylene chloride (200 mL) at 0° C. was added a solution of tetrabutylammonium dihydrogen trifluoride (51.4 g, 170.7 mmol) in methylene chloride (50 mL) dropwise. To this mixture was added NBS (30.3 g, 170.7 mmol). The reaction mixture was stirred for 18 h at room temperature and was then diluted with methylene chloride (100 mL). The mixture was washed with water (100 mL), brine (100 mL) and was then dried over anhydrous Na₂SO₄ and evaporated to dryness.

Flash chromatography (silica, EtOAc:hexanes 1:9) gave 1-trifluoromethyl-2,3-dihydro-1H-indole-5-carbonitrile as a pale yellow oil (4.0 g, 56%).

1H NMR (400 MHz, CDCl₃) δ 7.48-7.43 (2H, m), 7.04-7.02 (1H, m), 3.85-3.80 (2H, m), 3.18-3.13 (2H, m).

### Step 3:

To a stirred solution of 1-trifluoromethyl-2,3-dihydro-1H-indole-5-carbonitrile (4.0 g; 18.9 mmol) in CCl₄ (100 mL) at room temperature was added NBS (6.7 g, 37.7 mmol). The reaction mixture was stirred for 18 h at 80° C. The reaction mixture was diluted with methylene chloride (100 ml) and the resulting mixture was washed with water (100 mL) followed by brine (100 mL). The organic layers was dried over anhydrous Na₂SO₄ and evaporated to dryness. Flash chromatography (silica, EtOAc:hexanes 15:85) gave 3-bromo-1-trifluoromethyl-1H-indole-5-carbonitrile as a pale yellow solid (5.0 g, 93%).

To a solution of 3-bromo-1-trifluoromethyl-1H-indole-5-carbonitrile (500 mg; 1.73 mmol) in MeOH.NH₃ (20 mL, 5M) at room temperature was added 10% Pd/C (100 mg). The reaction mixture was stirred for 2 h at room temperature under H₂ (60 psi). The reaction mixture was then filtered through a plug of celite and the filtrate was evaporated to dryness to give 1-trifluoromethyl-1H-indole-5-carbonitrile as a off-white solid (350 mg, 97%).

1H NMR (400 MHz, CDCl₃) δ 7.99 (1H, s). 7.68-7.66 (1H, m), 7.60-7.57 (1H, m), 7.44-7.43 (1H, m), 6.75-6.74 (1H, m).

### Step 4:

To a solution of 1-trifluoromethyl-1H-indole-5-carbonitrile (350 mg, 1.67 mmol) in MeOH.NH₃ (20 mL, 5M) at room temperature was added Raney-Ni (100 mg). The reaction mixture was stirred for 2 h under H₂ (60 psi). The reaction mixture was filtered through a plug of celite and the filtrate evaporated to dryness. The crude compound was washed with pentane to give C(1-trifluoromethyl-1H-indol-5-yl)-methylamine IM17 as a white solid (0.200 g, 57%).

1H NMR (400 MHz, CDCl₃) δ 8.22 (2H, broad), 7.81-7.80 (2H, m), 7.68-7.65 (1H, m), 7.50-7.47 (1H, m), 6.89-6.88 (1H, m), 4.11 (2H, s).

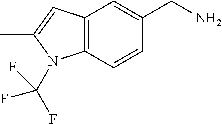

### IM18: C-(2-Methyl-1-trifluoromethyl-1H-indol-5-yl)-methylamine

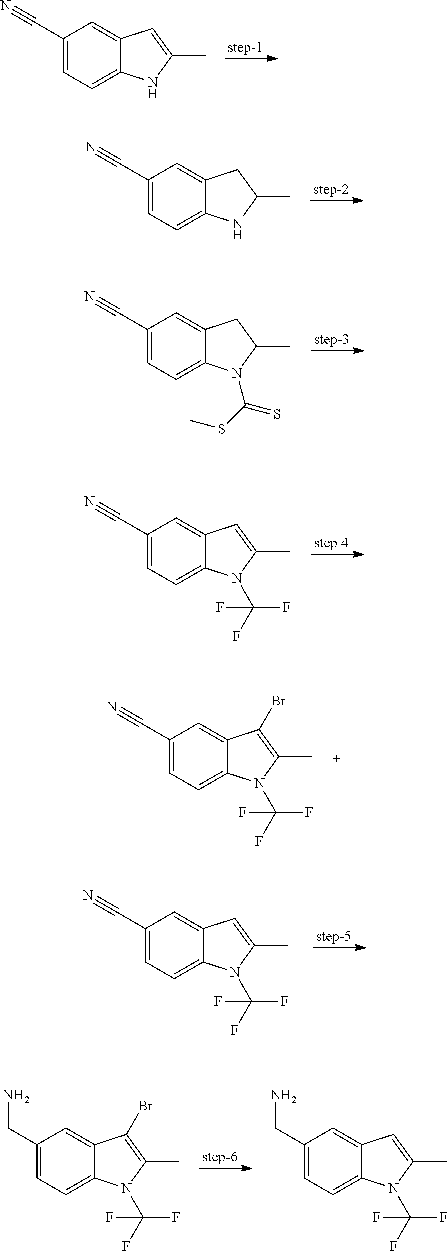

IM18 was prepared in a sequence of steps similar to IM17 and IM16 as outlined above. IM18 was isolated from HCl in isopropylacetate to give the HCl salt of the title compound as colorless crystals.

1H NMR (DMSO, 400 MHz) δ 8.31 (3H, br), 7.67 (1H, s), 7.61-7.58 (1H, m), 7.39-7.37 (1H, m), 6.65 (1H, s), 4.08 (2H, s), 2.50 (3H, s).

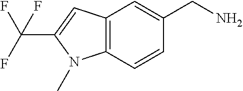

### IM19: C-(1-Methyl-2-trifluoromethyl-1H-indol-5-yl)-methylamine

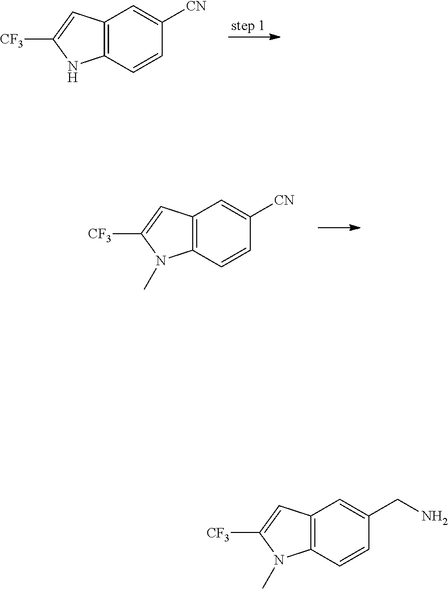

### Step 1:

NaH (0.71 g, 0.029 mol) was added to a solution of 2-trifluoromethyl-1H-Indole-5-carbonitrile (preparation described in WO2009/127678A1) (2.5 g; 0.011 mol) in DMF (50 mL) at room temperature. The reaction mixture was cooled to 0° C., MeI was added drop-wise and the reaction mixture was slowly warmed to room temperature. After stirring for 4 h the reaction mixture was poured into ice water. The mixture was filtered and the remanence was dried to give 1-methyl-2-trifluoromethyl-1H-indole-5-carbonitrile as a pale yellow solid (2.1 g, 79%).

### Step 2:

Raney Ni (400 mg) was added to solution of 1-methyl-2-trifluoromethyl-1H-indole-5-carbonitrile (2.0 g, 8.92 mmol) in MeOH.NH₃ (10M, 50 mL) under a N₂ atmosphere. The reaclion mixture was hydrogenated at 60 psi H₂ at room temperature for 4 h: The reaction mixture was tittered through a pad of celite and evaporated to dryness. The crude material was triturated with diethyl ether to give the title compound IM19 as a white solid (1.6 g, 79%). The compound was converted to its HCl salt using HCl in isopropyl acetate (4.0 M; 5.0 mL).

1HNMR (DMSO, 400 MHz) δ 8.24 (3H, br s), 7.79 (1H, s), 7.72-7.70 (1H, d), 7.49-7.47 (1H, d), 7.18 (1H, s), 4.11 (2H, s), 3.88 (3H, s).

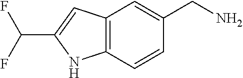

### IM20: C-(2-difluoromethyl-1H-indol-5-0)-methylamine

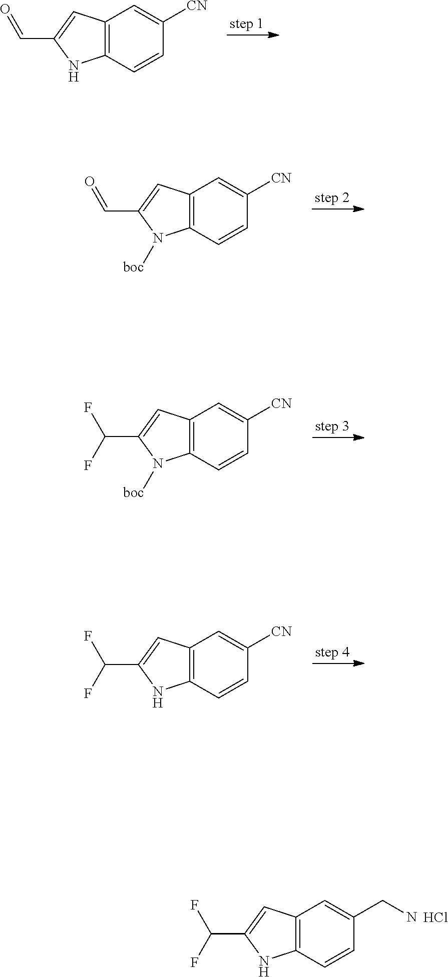

### Step 1:

To a solution of 2-formyl-1H-indole-5-carbonitrile (prepared as described in WO2009153307A1) (3.6 g, 21.17 mmol) in CH₃CN:THF (120 mL:120 mL) at 0° C. was added (Boc)₂O (6.0 g, 27.52 mmol) and DMAP (516 mg, 4.234 mmol). Cooling was removed and the mixture was stirred at 25° C. overnight. The reaction mixture was poured into ice-cold water (100 mL) and extracted with ethyl acetate (2×200 mL). The combined organic layers were dried over anhydrous Na₂SO₄ and evaporated to dryness. Flash chromatography (silica, EtOAc:petroleum ether 1:4) gave 5-cyano-2-formyl-indole-1-carboxylic acid tert-butyl ester as an off-white solid (2.6 g, 45%).

### Step 2:

To a solution of 5-cyano-2-formyl-indole-1-carboxylic acid tert-butyl ester (2.3 g, 8.51 mmol) in methylene chloride (200 mL) was slowly added deoxo-flour (bis(2-methoxyethyl)aminosulfur trifluoride) (7.53 g, 34.07 mmol) at 0° C. under N₂. The mixture was subsequently stirred at 25° C. overnight. The reaction mixture was then poured into a cold solution of saturated aqueous NaHCO₃ (100 mL). The aqueous layer was extracted with EtOAc (2×300 mL). The combined organic layers were dried over anhydrous Na₂SO₄ and evaporated to dryness. Flash chromatography (silica, EtOAc:petroleum ether 1:10) gave 5-cyano-2-difluoromethyl-indole-1-carboxylic acid tert-butyl ester as an off-white solid (1.8 g, 72%).

### Step 3:

To a solution of 5-cyano-2-difluoromethyl-indole-1-carboxylic acid tert-butyl ester (1.8 g, 6.16 mmol) in methylene chloride (60 mL) was slowly added trifluoro acetic acid (15 mL) at 0° C. and the reaction temperature was slowly increased to 25° C. and stirred at this temperature for 12 h. The mixture was then diluted with methylene chloride (100 mL) and washed with saturated aqueous NaHCO₃ (2×100 ml). The combined organic layers were dried over Na₂SO₄ and evaporated to dryness. Flash chromatography (silica, EtOAc:petroleum ether 1:4) gave 2-difluoromethyl-1H-indole-5-carbonitrile as an off-white solid (0.85 g, 72%).

### Step 4:

To a solution of 2-difluoromethyl-1H-indole-5-carbonitrile (3.0 g, 15.6 mmol) in methanol (40 mL) was carefully added Raney Ni (0.50 g) followed by 5 M ammonia in methanol (20 mL). The reaction mixture was hydrogenated at 60 psi for 12 h. The mixture was filtered and the filtrate evaporated to dryness. The crude material was dissolved in EtOAc (50 mL) and cooled to 0° C. A solution of 2M HCl in diethyl ether (5.0 mL) was added and the mixture stirred for 2 h. Filtration afforded the title compound IM20 as the HCl salt as a reddish solid (1.95 g, 64%).

1H NMR (DMSO 400 MHz) δ 11.94 (1H, s), 8.33 (3H, br), 7.73 (1H; s), 7.48-7.46 (1H, d), 7.37-7.09 (2H, m), 6.81-6.81 (1H, d), 4.07-4.06 (2H, m).

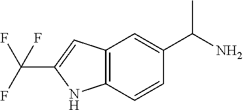

### IM21: (+)-1-(2-Trifluoromethyl-1H-indol-5-yl)-ethylamine

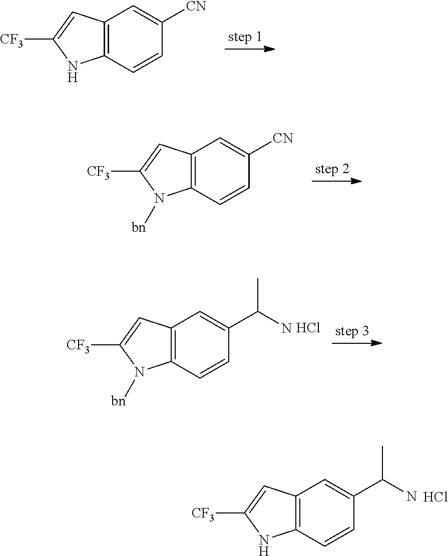

### Step 1:

To a solution of 2-trifluoromethyl-1H-indole-5-carbonitrile (5.0 g, 0.023 mol) in DMF (50 mL), was added NaH (2.8 g, 0.071 mol) at 0° C. Benzyl bromide (4.8 mL, 0.035 mol) was added drop-wise. The mixture was stirred overnight. The reaction mixture was poured in to ice-cold water and extracted with ethyl acetate (2×75 mL). The combined organic layers were washed with brine (2×50 mL), dried over Na₂SO₄ and evaporated to dryness. Flash chromatography (silica, EtOAc:petroleum ether 1:9) gave 1-benzyl-2-trifluoromethyl-1H-indole-5-carbonitrile as an oil that slowly solidified upon standing (5.0 g, 70%).

### Step 2:

To a stirred solution of 1-benzyl-2-trifluoromethyl-1H-indole-5-carbonitrile (4.0 g, 13.3 mmol) in dry toluene (10 mL) at 0° C. was added MeMgBr(1.4 M in toluene, 48 mL, 66.6 mmol) dropwise. After addition was completed the mixture was refluxed for 1 h. The mixture was cooled to 0° C. and then quenched with methanol. The resulting mixture was stirred at room temperature for 30 min and filtered. The remanence was washed with methanol (20 mL). The combined filtrates were cooled to 0° C. and NaBH₄ (4.0 g, 66.6 mmol) was added in small portions. Cooling was then removed and the mixture was left stirring at room temperature overnight. The mixture was quenched at 0° C. with saturated aqueous NH₄Cl and then extracted with EtOAc (2×100 mL). The combined organic layers were washed with brine, dried over Na₂SO₄ and evaporated to dryness. Flash chromatography (neutral alumina, MeOH:methylene chloride 1:9) gave 1.5 g of free base. This material was converted to its HCl salt by using 2 N HCl in ether (10 mL) to give 1-(1-Benzyl-2-trifluoromethyl-1H-indol-5-yl)ethylamine HCl salt (2.0 g, 35%).

Chiral separation by SFC (Method F) of racemic 1-(1-Benzyl-2-trifluoromethyl-1H-indol-5-yl)ethylamine (5.5 g) gave 2.4 g (−) isomer as peak 1 (specific optical rotation −13.3°, 0.5% MeOH) and 1.1 g (+) isomer as peak 2 (specific optical rotation +11.5°, 0.5% MeOH).

### Step 3:

To a stirred solution of (+)-1-(1-benzyl-2-trifluoromethyl-1H-indol-5-yl)-ethylamine (1.1 g, 3.46 mmol) in DMSO (11 mL) was added t-BuOK in THF (1 M, 28 mL, 28 mmol) drop-wise at 0° C. Oxygen gas was bubbled through the reaction mixture for 15 min. The mixture was then stirred for 30 min. The reaction mixture was then slowly poured into crushed ice. The resulting mixture was extracted with EtOAc (2×75 ml) and the combined organic layers were washed with brine and evaporated to dryness. The crude product was dissolved in diethyl ether (10 mL). To this solution was added 2 M HCl in diethyl ether (4 mL) and the mixture was stirred for 5 min and evaporated to dryness. The resulting salt was washed with 10% methanol in methylene chloride (5 ml) and dried to give the title compound IM21 as the HCl salt (0.50 g, 55%).

1HMR (DMSO, 400 MHz) δ 12.42 (1H, s), 8.42 (3H, br s), 7.79 (1H, s), 7.54-7.52 (1H, d), 7.46-7.44 (1H, d), 7.07 (1H, s), 4.48 (1H, br s), 1.56-1.55 (3H, d).

(specific optical rotation +14.71°, 0.5% MeOH)

### IM22: (−)-1-(2-Trifluoromethyl-1H-indol-5-yl)-ethylamine

The (−)-1-(1-benzyl-2-trifluoromethyl-1H-indol-5-yl)-ethylamine isomer was taken the same step as described for IM21 to give (−)1-(2-Trifluoromethyl-1H-indol-5-yl)-ethylamine.

(specific optical rotation −17.22°, 0.5% MeOH)

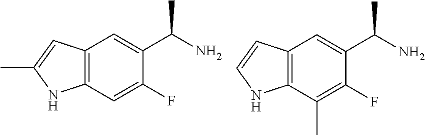

### IM23: (R)-1-(6-Fluoro-2-methyl-1H-indol-5-yl)-ethylamine and (R)-1-(6-Fluoro-7-methyl-1H-indol-5-yl)-ethylamine

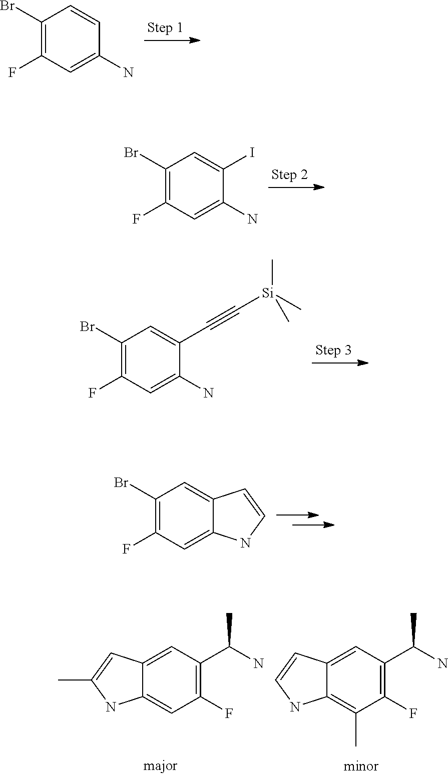

### Step 1:

To a solution of 4-bromo-3-fluoro-aniline (40.0 g, 211.6 mmol) in acetic acid (520 mL) was slowly added N-iodo succinimide (57.14 g, 253.9 mmol) 25° C. The mixture was stirred for 90 min. The reaction mixture was poured into ice-water and filtered. The remanence was dried in vacuo to give 4-bromo-5-fluoro-2-iodo-phenylamine as a brown solid (60 g, 90%).

### Step 2:

To a solution of 4-bromo-5-fluoro-2-iodo-phenylamine (30.0 g, 95.2 mmol), Pd(PPh₃)₂Cl₂ (3.3 g, 4.7 mmol) and copper iodide (0.9 g, 4.7 mmol) in triethylamine (700 mL) was slowly added trimethylsilytacetylene (9.3 g. 95.2 mmol) at 0° C. The mixture was subsequently stirred at 25° C. for 2 h. The mixture was then filtered through a pad of celite. The filtrate was washed with water and brine. The organic layer was dried over Na₂SO₄, and concentrated in vacuo. Flash chromatography (silica, EtOAc:petroleum ether 1:9) gave 4-bromo-5-fluoro-2-trimethylsilanylethynyl-phenylamine as a yellow oil (20 g, 74%).

### Step 3:

To a solution of 4-bromo-5-fluoro-2-trimethylsilanylethynyl-phenylamine (50.0 g, 175.4 mmol) in DMF (1000 mL) at 0° C. was added copper iodide (66.0 g, 351 mmol). The reaction mixture was stirred at 100° C. for 4 h. The reaction mixture was then diluted with water (1000 mL) and extracted with EtOAc (2×1000 mL). The combined organic layers were dried over anhydrous Na₂SO₄ and evaporated to dryness. Flash chromatography (silica, EtOAc:petroleum ether 1:9) gave 5-bromo-6-fluoro-1H-indole as a brown solid (20 g, 54%).

1H NMR (DMSO, 400 MHz) δ 11.32 (1H, s), 7.84-7.82 (1H, d), 7.41-7.35 (2H, m), 6.43-6.42 (1H, m).

The 5-bromo-6-fluoro-1H-indole was taken through steps 2 to 8 as described for IM28. The methylation step 3 gave two methylated isomers with 2-methyl being major and 7-methyl being minor. This binary mixture was used without further purification to make amides 102 and 103.

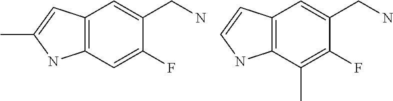

### IM24: C-(6-Fluoro-2-methyl-1H-indol-5-yl)-methylamine and C-(6-Fluoro-7-methyl-1H-indol-5-yl)-methylamine

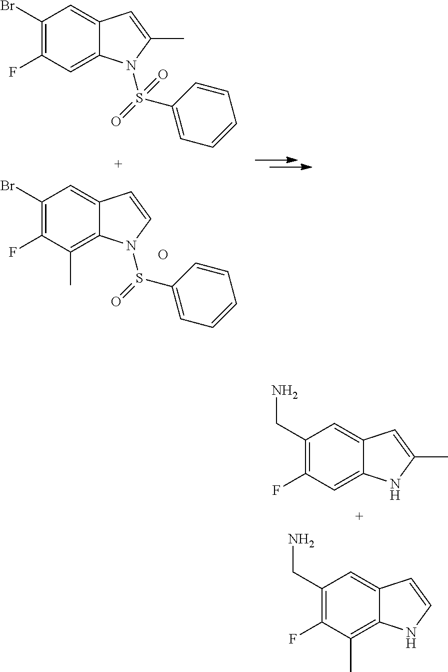

5-bromo-6-fluoro-1H-indole was taken through steps 2 to 3 as described for IM28 to give a mixture of two methylated indoles. This binary mixture was taken through steps 1 to 3 as described for IM26. The resulting binary mixture was used without further purification to make amides 104 and 105

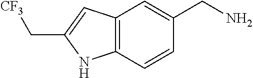

### IM25: C-[2-(2.2.2-Trifluoro-ethyl)-1H-indol-5-yl]-methylamine

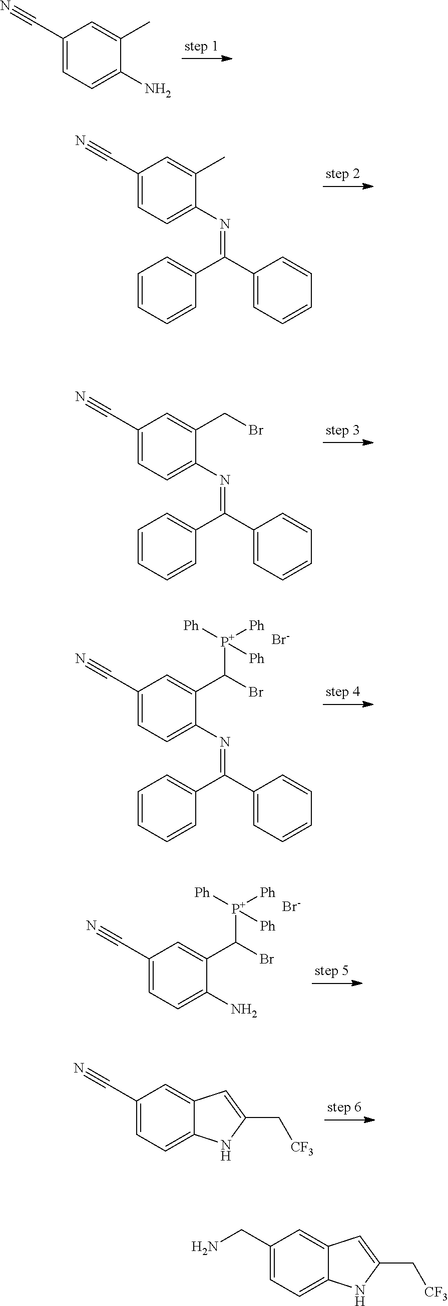

### Step 1:

To a solution of 4-amino-3-methylbenzonitrile 1 (5.0 g, 37.87 mmol) in toluene (50 mL) was added benzophenone (7.5 g, 45.45 mmol) and pTSA (0.07 g, 0.378 mmol) at room temperature. The flask was fitted with dean-stark trap and heated to reflux for 7 days. The mixture was evaporated to dryness. Flash chromatography (silica, EtOAc:petroleum ether 4:96) gave 4-(benzhydrylidene-amino)-3-methyl-benzonitrile as a yellow solid (8.0 g, 71%).

### Step 2:

To a solution of 4-(benzhydrylidene-amino)-3-methyl-benzonitrile (7.0 g, 23.64 mmol) in CCl₄ (75 mL) was added N-bromo succinimide (4.6 g, 26.0 mmol) and benzoyl peroxide (0.8 g, 3.54 mmol). The mixture was heated to reflux for 6 h. The mixture was then diluted with 1N NaOH solution (100 mL) and extracted with methylene chloride (2×100 mL). The combined organic layers were washed with brine, dried over Na₂SO₄ evaporated to dryness. Flash chromatography (silica, EtOAc:petroleum ether 3:97) gave 4-(benzhydrylidene-amino)-3-bromomethyl-benzonitrile as a yellow solid (6.0 g, 68%).

### Step 3:

To a solution of 4-(benzhydrylidene-amino)-3-bromomethyl-benzonitrile (500 mg, 1.34 mmol) in toluene (15 mL) was added triphenyl phosphine (420 mg, 1.61 mmol). The mixture was heated to reflux for 12 h. The mixture was diluted with EtOAc (20 mL) and stirred for 15 min and then filtered. The remanence was washed with EtOAc and dried under vacuum to give [6-(benzhydrylidene-amino)-3-cyano-cyclohexa-1,3,4,5-tetraenylmethyl]-triphenyl-phosphonium; bromide as an off-white solid (0.60 g, 81%) which was used without further purification.

### Step 4:

To a solution of [6-(benzhydrylidene-amino)-3-cyano-cyclohexa-1,3,4,5-tetraenylmethyl]-triphenyl-phosphonium; bromide (250 mg, 0.449 mmol) in THF:methylene chloride (2 mL: 2 mL) at 0° C. was added HCl in diethyl ether(1M, 1 mL). The mixture was subsequently stirred at room temperature for 12 h. The mixture was then filtered and the remanence was washed with EtOAc and dried under vacuum to give (6-amino-3-cyano-cyclohexa-1,3,4,5-tetraenylmethyl)-triphenyl-phosphonium; bromide as an off-white solid (0.15 g, 85%) which was used without further purification.

### Step 5:

To a solution of (6-amino-3-cyano-cyclohexa-1,3,4,5-tetraenylmethyl)-triphenyl-phosphonium; bromide (4.0 g, 10.2 mmol) in THF (200 mL) was added 3,3,3-trifluoro propanoic acid (1.35 mL, 15.3 mmol), propyl phosphonic anhydride (50% in DMF) (14.2 g, 44.88 mmol) and DIPEA (6.7 mL, 40.8 mmol) at room temperature. The mixture was stirred at this temperature for 6 h. The volatiles were removed in vacuo and the remanence was taken up in DMF (120 mL) and heated to reflux for 48 h. The reaction mixture was then diluted with water (100 mL) and extracted with EtOAc (2×100 mL). The combined organic layers were washed with brine, dried over Na₂SO₄ and evaporated to dryness. Flash chromatography (silica, EtOAc:petroleum ether 1:4) gave 2-(2,2,2-trifluoro-ethyl)-1H-indole-5-carbonitrile as colorless crystals (0.11 g, 5%).

### Step 6:

To a solution of 2-(2,2,2-trifluoro-ethyl)-1H-indole-5-carbonitrile (110 mg, 0.491 mmol) in methanol (3 mL) was added Raney Ni (20 mg) followed by 5 M ammonia in methanol (2 mL). The reaction solution was hydrogenated at 60 psi for 4 h at room temperature. The reaction mixture was filtered through a plug of celite. The filtrate was concentrated under vacuo and the resulting crystals were washed with diethyl ether to give the title compound IM25 as colorless crystals (0.11 g, 98%).

1H NMR (DMSO, 400 MHz) δ 11.06 (1H, s), 7.41 (1H, s), 7.29-7.27 (1H, m), 7.46-7.42 (1H, m), 6.34 (1H, s), 3.83-3.75 (4H, m), 2.23 (2H, br s).

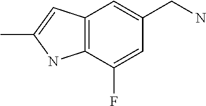

### IM26: C-(7-Fluoro-2-methyl-1H-indol-5-yl)-methylamine

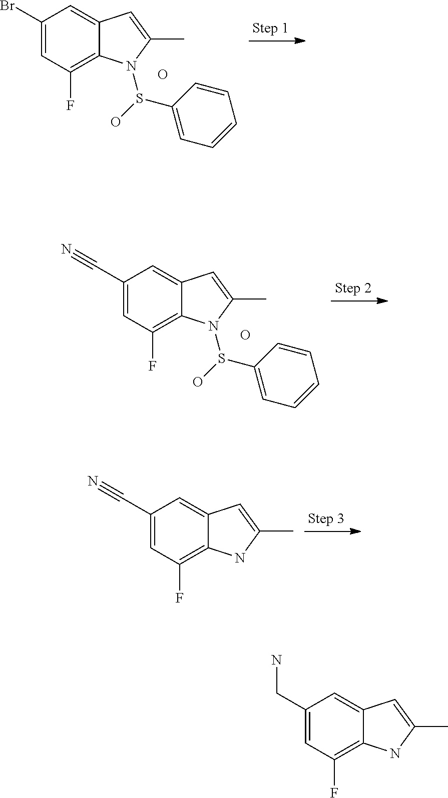

### Step 1:

To a solution of 1-benzenesulfonyl-5-bromo-7-fluoro-2-methyl-1H-indole (prepared as described for IM28 (2.0 g; 5.44 mmol) in DMF (40 mL) was added CuCN (975 mg; 10.8 mmol) at room temperature. The reaction temperature was slowly heated to 150° C. and stirred at this temperature for 12 h. The mixture was then cooled to room temperature and diluted with EtOAc (100 mL). The mixture was filtered through a pad of celite and the filtrate was washed with water followed by brine. The organic layer was dried over anhydrous Na₂SO₄ and evaporated to dryness. Flash chromatography (silica, EtOAc:petroleum ether 15:85) gave 1-benzenesulfonyl-7-fluoro-2-methyl-1H-indole-5-carbonitrile as an off-white solid (1.0 g, 58%).

### Step 2:

To a vigorously stirring solution of 1-benzenesulfonyl-7-fluoro-2-methyl-1H-indole-5-carbonitrile (1.0 g, 3.18 mmol) in THF: MeOH (20 mL:20 mL) at 0° C. was added 10 N NaOH (4.7 mL) dropwise. The mixture was stirred for 30 min at 0° C. and then left without cooling overnight. The mixture was evaporated to dryness and the resulting residue was diluted with water (20 mL) and stirred for 30 min. The formed solid was collected by filtration and washed with water. Flash chromatography (silica, EtOAc:petroleum ether 1:4) gave 7-fluoro-2-methyl-1H-indole-5-carbonitrile as an off-white solid (400 mg, 72%).

### Step 3:

To a solution of 7-fluoro-2-methyl-1H-indole-5-carbonitrile (400 mg, 2.29 mmol) in methanol (4 ml) was carefully added Raney Ni (100 mg) followed by 5 M ammonia in methanol (6 mL). The reaction solution was hydrogenated at 60 psi for 4 h. The mixture was filtered through a pad of celite and the filtrate was evaporated to dryness. The crude compound was purified by washing with ether to give the title compound IM26 as a white solid (350 mg, 86%).

1H NMR (DMSO, 400 MHz) δ 11.18 (1H, s), 7.11 (1H, s), 6.81-6.78 (1H, m), 6.12 (1H, s), 3.72 (2H, br s), 2.36 (3H, s), 1.75 (2H, br s).

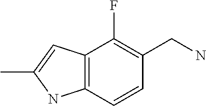

### IM27: C-(4-Fluoro-2-methyl-1H-indol-5-yl-methylamine

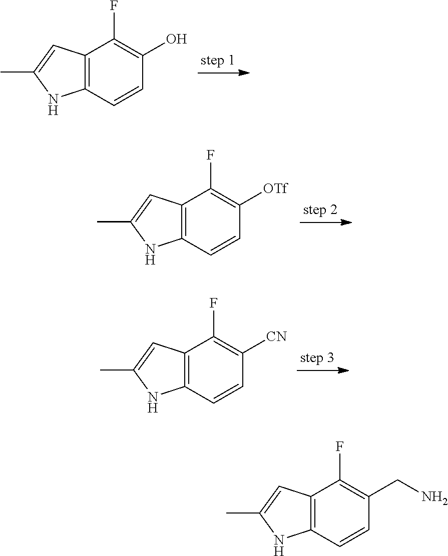

### Step 1:

### 2,6-lutidine (1.2 g, 10.9 mmol) was added to a mixture of 4-fluoro-2-methyl-1H-indol-5-ol (1.4 g, 9.1 mmol) in methylene chloride (70 ml) at 0° C. To this mixture was added Tf₂O (3.0 g, 10.9 mmol) dropwise and the mixture was stirred at room temperature overnight. Water was added and the mixture was extracted with methylene chloride (1500 mL). The organic layer was washed with water, brine, dried over Na₂SO₄ and evaporated to dryness. Flash chromatography (silica, EtOAc:petroleum ether 1:10) gave trifluoro-methanesulfonic acid 4-fluoro-2-methyl-1H-indol-5-yl ester as a yellow solid (1.9 g, 70%).

### Step 2:

A round bottomed flask was charged with trifluoro-methanesulfonic acid 4-fluoro-2-methyl-1H-indol-5-yl ester (500 mg, 1.7 mmol), Pd₂(dba), (183 mg, 0.2 mmol), dppf (440 mg, 0.8 mmol), Zn powder (30 mg, 0.4 mmol) and Zn(CN)₂ (200 mg, 1.7 mmol) and degassed and backfilled with N₂ three times. DMF (10 mL) was added via syringe and the reaction mixture was stirred at 120° C. for 5 h. The mixture was cooled to room temperature and diluted with water. The mixture was then extracted with EtOAc (100 mL), dried over Na₂SO₄ and evaporated to dryness. Flash chromatography (silica, EtOAc:petroleum ether 1:20 to 1:5) gave 4-fluoro-2-methyl-1H-indole-5-carbonitrile as a yellow solid (255 mg, 86%). 1H NMR (CDCl₃, 400 MHz) 8.37 (s, 1H), 7.25-7.12 (m, 2H), 6.40 (t, 1H), 2.48 (s, 3H).

### Step 3:

Raney Ni (300 mg) was added to a mixture of 4-fluoro-2-methyl-1H-indole-5-carbonitrile (400 mg, 2.3 mmol) in MeOH (100 mL) and NH₃.H₂O (10 mL). The reaction mixture was stirred at room temperature overnight under 50 psi of H₂. The mixture was filtered and evaporated to dryness to give the title compound IM27 as a yellow solid (390 mg, 95%) which was used without further purification.

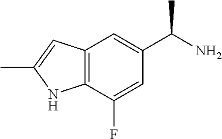

### IM28: (R)-1-(7-Fluoro-2-methyl-1H-indol-5-yl)-ethylamine

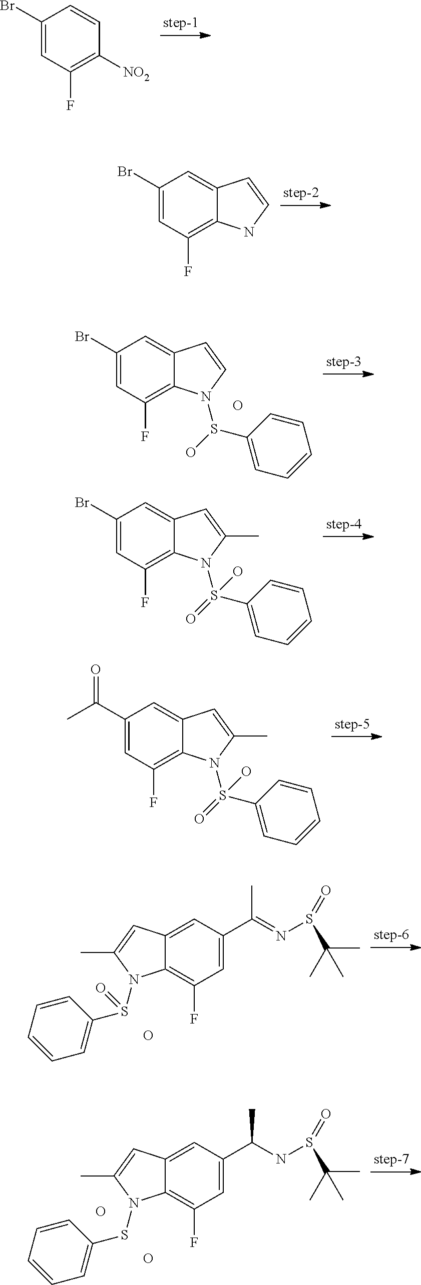

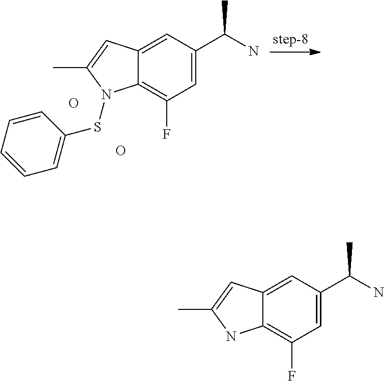

### Step 1:

To a solution of 4-bromo-2-fluoro-1-nitrobenzene (1.0 g, 4.54 mmol) in THF (20 mL) was added vinyl magnesium bromide (1M in THF, 13.62 mL, 13.62 mmol) slowly at −40° C. The reaction mixture was maintained at this temperature for 60 min. After completion of the reaction saturated aqueous NH₄Cl solution was added and the mixture was extracted with EtOAc (2×20 mL). The combined organic layers were dried over Na₂SO₄ and evaporated to dryness. Flash chromatography (silica, EtOAc:petroleum ether 9:1) gave 5-bromo-7-fluoro-1H-indole as a gummy solid (0.24 g, 25%).

### Step 2:

To a suspension of NaH (0.112 g, 2.8 mmol) in THF (2 mL) at 0° C. was added a solution of 5-bromo-7-fluoro-1H-indole (200 mg, 0.934 mmol) in THF (2 mL) dropwise. The reaction mixture was stirred for 10 min. Benzenesulfonyl chloride (297 mg, 1.68 mmol) dissolved in THF (2 mL) was added dropwise. The mixture was slowly warmed to room temperature over 2 h while stirring. After completion of the reaction the reaction mixture was poured into ice-water (10 mL). The resulting mixture was filtered and the remanence was washed with water and petroleum ether and then dried in vacuo to give 1-benzenesulfonyl-5-bromo-7-fluoro-1H-indole as an off-white solid (0.15 g, 43%).

### Step 3:

A solution of LDA in THF (2.0M, 35.1 mL, 68.8 mmol) added to THF (120 mL) and the mixture was cooled to −78° C. A solution of 1-benzenesulfonyl-5-bromo-7-fluoro-1H-indole (13.5 g, 38.2 mmol) in THF (90 mL) added dropwise. After 30 min the mixture was allowed to reach −30° C. and stirred at this temperature for 30 min before being re-cooled to −78° C. Iodomethane (5.4 mL, 84.04 mmol) in THF (50 mL) was added dropwise and the mixture was allowed to slowly reach room temperature overnight. The mixture was quenched with saturated aqueous NH₄Cl solution and the resulting mixture was extracted with EtOAc (2×200 mL). The combined organic layers were dried over Na₂SO₄ and evaporated to dryness. Flash chromatography (silica, EtOAc:petroleum ether 6:94) gave 1-benzenesulfonyl-5-bromo-7-fluoro-2-methyl-1H-indole as an off-white solid (7.4 g, 53%).

### Step 4:

To a solution of 1-benzenesulfonyl-5-bromo-7-fluoro-2-methyl-1H-indole (5.0 g, 13.62 mmol) in THF (100 mL) was slowly added t-BuLi (1.5M in heptanes, 16.3 mL, 24.5 mmol) at −78° C. The mixture was stirred for 30 min. N-methoxy-methyl acetamide (2.8 mL, 27.2 mmol) was added to reaction mixture and the mixture was left without cooling overnight. The reaction was quenched with aqueous saturated NH₄Cl solution and extracted with ethyl acetate (2×20 mL). The combined organic layers was dried over Na₂SO₄ and evaporated to dryness. Flash chromatography (silica, EtOAc:petroleum ether 15:85) gave 1-(1-benzenesulfonyl-7-fluoro-2-methyl-1H-indol-5-yl)-ethanone as colorless crystals (1.3 g, 29%).

### Step 5:

To a solution of 1-(1-benzenesulfonyl-7-fluoro-2-methyl-1H-indol-5-yl)-ethanone (1.3 g, 3.92 mmol) in THF (30 mL) was added (S)-2-methyl-2-propanesulfinamide (714 mg, 5.89 mmol) followed by Ti(OEt)₄ (1.65 mL, 7.84 mmol) at room temperature. The reaction mixture was stirred at reflux for 12 h. The mixture was diluted with brine (30 mL) and extracted with EtOAc (2×50 mL). The combined organic layers were dried over Na₂SO₄ and evaporated dryness. Flash chromatography (silica, EtOAc:petroleum ether 40:60) gave (S)-2-methyl-propane-2-sulfinic acid [1-(1-benzenesulfonyl-7-fluoro-2-methyl-1H-indol-5-yl)-ethylidene]-amide (1.3 g, 76%).

### Step 6:

To a solution of (S)-2-methyl-propane-2-sulfinic acid [1-(1-benzenesulfonyl-7-fluoro-2-methyl-1H-indol-5-yl)-ethylidene]-amide (1.3 g, 2.99 mmol) in THF (30 mL) was slowly added L-selectride (1.0M in THF, 3.89 mL, 3.89 mmol) at −78° C. The reaction mixture was stirred at this temperature for 2 h. The mixture was quenched with saturated, aqueous NH₄Cl solution and extracted with ethyl acetate (2×30 mL). The combined organic layers were dried over Na₂SO₄ and evaporated to dryness to give (S)-2-methyl-propane-2-sulfinic acid [(R)-1-(1-benzenesulfonyl-7-fluoro-2-methyl-1H-indol-5-yl)-ethyl]-amide (1.2 g, 92%).

### Step 7:

To a solution of (S)-2-methyl-propane-2-sulfinic acid [(R)-1-(1-benzenesulfonyl-7-fluoro-2-methyl-1H-indol-5-yl)-ethyl]-amide (1.2 g, 2.75 mmol) in methanol (24 mL) was added 6 N HCl (12 mL) at 0° C. The reaction mixture was stirred at 25° C. for 6 h and then evaporated to dryness. Water was added to the crude product and the mixture was extracted with EtOAc (2×30 mL). The combined organic layers were dried over Na₂SO₄ and evaporated to dryness to give (R)-1-(1-benzenesulfonyl-7-fluoro-2-methyl-1H-indol-5-yl)-ethylamine as an off-white solid (0.80 g, 88%).

### Step 8:

To a solution of (R)-1-(1-benzenesulfonyl-7-fluoro-2-methyl-1H-indol-5-yl)-ethylamine (800 mg, 2.40 mmol) in THF: MeOH (8 mL:8 mL) at 0° C. was added 10 N NaOH (3.6 mL) dropwise. The mixture was stirred at this temperature for 30 min and then at room temperature for 48 h. The mixture evaporated to dryness the resulting residue was diluted with water (20 mL). The mixture was extracted with EtOAc (2×20 mL). The combined organic layers were dried over Na₂SO₄ and evaporated to dryness. Flash chromatography (silica, MeOH:methylene chloride 3:7) gave the title compound IM28 as an off-white solid (300 mg, 65%).

1H NMR (DMSO, 400 MHz) δ 11.49 (1H, s), 8.33 (3H, br s), 7.34 (1H, s), 7.05-7.02 (1H, d), 6.23 (1H, s), 4.43-4.38 (1H, m), 2.39 (3H, s), 1.53-1.52 (3H, m).

### Preparation of Carboxylic Acid Derivatives

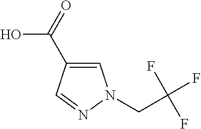

### IM29: 1-(2,2,2-Trifluoro-ethyl)-1H-pyrazole-4-carboxylic acid

The compound can be prepared as described in WO2009010871A2.

1H NMR (DMSO, 400 MHz) δ 12.57 (br s, 1H), 8.37 (s, 1H), 7.93 (s, 1H), 5.21-5.16 (m, 2H).

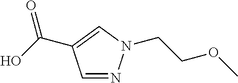

### IM30: 1-(2-Methoxy-ethyl)-1H-pyrazole-4-carboxylic acid

The compound was prepared as described for IM31 using 2-methoxy-1-bromoethane to give the title compound IM30 as a colorless solid.

1H NMR (DMSO, 400 MHz) δ 12.28 (1H, br, s), 8.19 (1H, s), 7.79 (1H, s), 4.30-4.27 (2H, m), 3.69-3.67 (2H, m), 3.22 (3H, s).

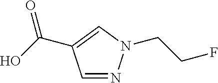

### IM31: 1-(2-Fluoro-ethyl)-1H-pyrazole-4-carboxylic acid

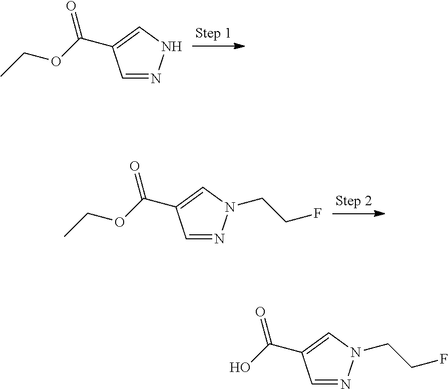

### Step 1:

To a solution of 1H-pyrazole-4-carboxylic acid ethyl ester (4.3 g, 30.68 mmol) in CH₃CN (30 mL) was added K₂CO₃ (6.35 g, 46.02 mmol) and 2-fluoro-1-bromo-ethane (4.28 g, 33.75 mmol). The reaction mixture was stirred for 48 h at room temperature. The mixture was filtered and the filtrate was evaporated to dryness. Flash chromatography (silica, EtOAc:petroleum ether 1:10) gave 1-(2-fluoro-ethyl)-1H-pyrazole-4-carboxylic acid ethyl ester as an off-white solid (4.0 g, 70%).

### Step 2:

To a solution of 1-(2-fluoro-ethyl)-1H-pyrazole-4-carboxylic acid ethyl ester (4.0 g, 21.48 mmol) in THF: CH₃OH:water (20:60:20 mL) was added LiOH (1.8 g, 42.96 mmol) and the mixture was stirred at room temperature for 6 h. The volume was concentrated under reduced pressure and pH was adjusted to 2-3 using 1N HCl. The mixture was extracted with EtOAc (3×50 mL) and the combined organic layers were washed with water (2×30 mL), dried over Na₂SO₄ and evaporated to dryness to give the title compound IM31 as a colorless solid (2.3 g, 67%).

1H NMR (DMSO, 400 MHz) δ 12.34 (1H, s), 8.28 (1H, s), 7.84 (1H,$), 4.83-4.85 (1H, m), 4.71-4.73 (1H, m), 4.49-4.52 (1H, m), 4.43-4.45 (1H, m).

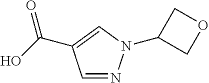

### IM32: 1-Oxetan-3-yl-1H-pyrazole-4-carboxylic acid

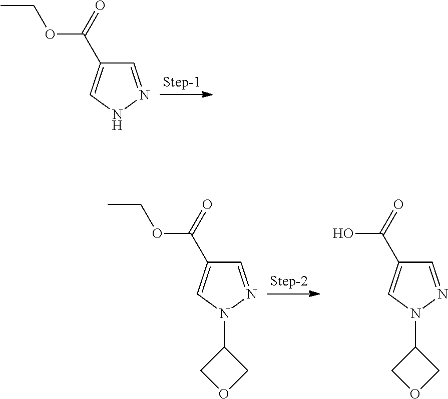

### Step 1:

To a solution of 1H-pyrazole-4-carboxylic acid ethyl ester (1.0 g, 7.13 mmol) in DMF (10 mL) was added Cs₂CO₃ (6.97 g, 21.40 mmol) and 3-bromo-oxetane (1.07 g, 7.84 mmol) and the mixture was stirred for 16 h at 100° C. The mixture was then quenched with water and extracted with EtOAc (3×20 mL). The combined organic layers were dried over Na₂SO₄ and evaporated to dryness to give 1-oxetan-3-yl-1H-pyrazole-4-carboxylic acid ethyl ester as a colorless oil (1.0 g, 64%) which was used without further purification.

### Step 2:

To a solution of 1-oxetan-3-yl-1H-pyrazole-4-carboxylic acid ethyl ester (1.7 g, 8.66 mmol) in THF:CH₃OH:Water (10:30:10 mL) was added LiOH (0.72 g, 17.32 mmol) at room temperature. The mixture was stirred for 4 h and then concentrated in vacuo. The pH was adjusted to 2-3 using 1N HCl. The mixture was extracted with EtOAc (3×30 mL), the combined organic layers were dried over Na₂SO₄ and evaporated to dryness. The remanence was washed with CH₃CN (10 mL) and filtered to give the title compound IM32 as a colorless solid (800 mg, 55%) which was used without further purification.

1H NMR (DMSO, 400 MHz) δ 12.39 (1H, br s), 8.37 (1H, s), 7.93 (1H, s), 5.65-5.58 (1H, m), 4.92-4.89 (2H, m), 4.88-4.86 (2H, m).

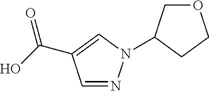

### IM33: 1-(Tetrahydro-furan-3-O-1H-pyrazole-4-carboxylic acid

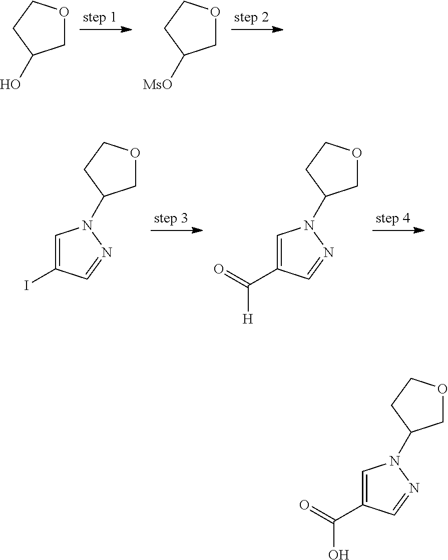

### Step 1:

To a solution of tetrahydro-furan-3-ol (9.0 g, 79 mmol) and triethyl amine (20 mL, 176 mmol) in THF (200 mL) was added methanesulfonyl chloride (12.9 g, 113 mmol) at 0° C. The mixture was stirred overnight and then evaporated to dryness. The residue was extracted with EtOAc (3×300 mL) and washed with saturated, aqueous NaRCO₃. The organic layer was dried over Na₂SO₄ and evaporated to dryness to give methanesulfonic acid tetrahydro-furan-3-yl ester as a yellow viscous liquid (12.4 g. 94%) which was used without further purification.

### Step 2:

To a solution of 4-iodo-1H-pyrazole (11.0 g, 56.7 mmol) in DMF (100 mL) was added NaH (2.50 g, 62.38 mmol, 60% in oil). The mixture was stirred at 25° C. for 30 min. A solution of methanesulfonic acid tetrahydro-furan-3-yl ester (10.4 g, 62.38 mmol) in DMF (20 mL) was added dropwise. The mixture was then stirred at 110° C. for 48 hours. This mixture was evaporated to dryness and purified by preparative HPLC (Method H) to give 4-iodo-1-(tetrahydro-furan-3-yl)-1H-pyrazole as a colorless oil (8.03 g, 53%).

### Step 3:

To a solution of 4-iodo-1-(tetrahydro-furan-3-yl)-1H-pyrazole (7.00 g, 26.8 mmol) in THF (100 mL) was added i-PrMgCl (2M in THF, 106.0 mL) dropwise at 0° C. The mixture was then stirred at 25° C. for 30 min. DMF (9.69 g, 132.5 mmol) was added and the mixture was stirred at room temperature overnight. The mixture was concentrated in vacuo and extracted with EtOAc (3×200 mL). The combined organic layers were washed with saturated, aqueous NH₄Cl (200 mL) and then evaporated to dryness. Flash chromatography (silica, EtOAc:petroleum ether 1:2) gave 1-(tetrahydro-turan-3-yl)-1H-pyrazole-4-carbaldehyde as a colorless solid (3.04 g, 60%).

### Step 4:

To a solution of 1-(tetrahydro-furan-3-yl)-1H-pyrazole-4-carbaldehyde (3.00 g, 18.05 mmol) in dioxane (150 mL) and H₂O (30 mL) was added KMnO₄ (3.0 g, 20 mmol) at 25° C. The mixture was stirred at room temperature for 5 hours and then evaporated to dryness. The remanence was washed with CH₃CN and EtOAc to give the title compound IM33 as colorless crystals (2.20 g, 67% yield) which was used in the next step without further purification.

1H NMR (DMSO 400 MHz) δ 7.69 (s, 1H), 7.44 (s, 1H), 4.95-4.89 (m, 1H), 3.96-3.90 (m, 2H), 3.84-3.77 (m, 2H), 2.37-2.28 (m, 1H), 2.25-2.17 (m, 1H).

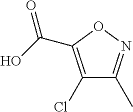

### IM34: 4-Chloro-3-methyl-isoxazole-5-carboxylic acid

The compound was prepared as described in WO 00/024739A1 to give IM34.

1H NMR: (CDCl₃ 400 MHz TMS): δ 8.59 (br s, 1H), 2.39 (s, 3H).

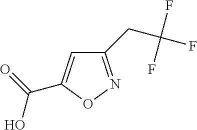

### IM35: 3-(2,2,2-Trifluoro-ethyl)-isoxazole-5-carboxylic acid

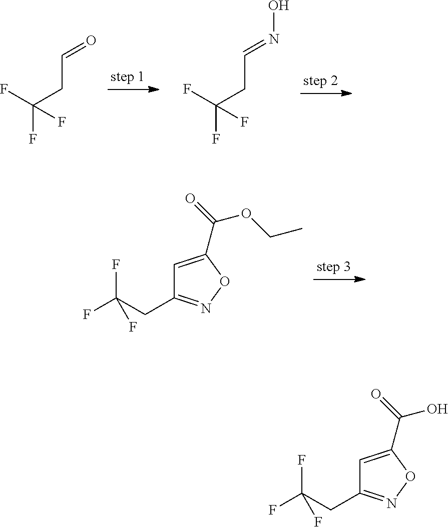

### Step 1:

To a stirring solution of aqueous NaOH (14.4 g in 140 mL of water, 1.5 eq, 2.57 M) was added NH₂OH.HCl (25.12 g, 361.57 mmol) portion wise at 20° C. To this mixture was added 3,3,3-trifluoro-propionaldehyde (27.0 g, 241.0 mmol) and the reaction mixture was stirred at room temperature for 2 h. The mixture was extracted with methylene chloride (4×100 mL). The combined organic layers were dried over Na₂SO₄ and evaporated to dryness to give the crude oxime as a white liquid (27 g) which was used without further purification.

### Step 2:

To a stirring solution of 3,3,3-trifluoro-propionaldehyde oxime (8.5 g, 66.9 mmol) in THF (178 mL) was added propynoic acid ethyl ester (8.9 mL, 133.8 mmol) at 20° C. A solution of 4% aqueous NaOCl (240 mL) was added and the reaction mixture was stirred at room temperature for 2 h. The mixture was then extracted with EtOAc (3×50 mL). The combined organic layers were dried over Na₂SO₄ and evaporated to dryness. Flash chromatography (silica, EtOAc:petroleum ether 7:93) gave 3-(2,2,2-trifluoro-ethyl)-isoxazole-5-carboxylic acid ethyl ester (2.3 g, 15%).

### Step 3:

To a solution of 3-(2,2,2-trifluoro-ethyl)-isoxazole-5-carboxylic acid ethyl ester (10.0 g, 44.8 mmol) in THF: MeOH: H₂O (40:120:40 mL) was added LiOH (1.07 g, 44.8 mmol). The mixture was stirred at room temperature for 16 h. The volatiles were removed under reduced pressure and water (100 mL) was added. The aqueous layer was washed with EtOAc (3×50 mL) and subsequently the pH was adjusted to 4-5 using acetic acid. The mixture was evaporated to dryness and dried by co-distillation with toluene (3×50 mL). The remanence was washed with diethyl ether (50 mL) to give the title compound IM36 as a colorless solid (5.2 g, 60%).

1H NMR (DMSO, 400 MHz) δ 6.41 (s, 1H), 3.76-3.84 (m, 2H).

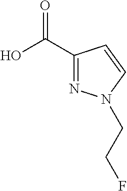

### IM36: 1-(2-Fluoro-ethyl)-1H-pyrazole-3-carboxylic acid

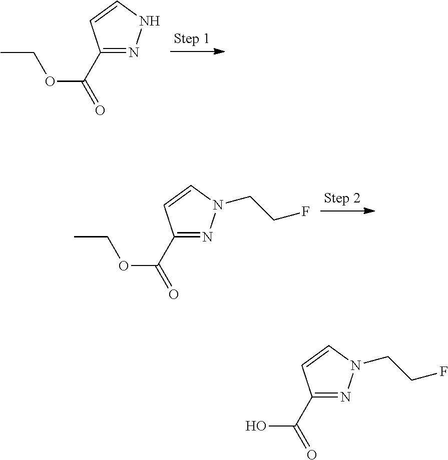

### Step 1:

To a suspension of 1H-pyrazole-3-carboxylic acid ethyl ester (12.0 g, 85.67 mmol) in THF (250 mL) at 0° C. was added NaH (3.08 g, 128.51 mmol) in small portions. The mixture was stirred for 30 min. Then 2-fluoro-1-bromo ethane (11.87 g, 94.24 mmol) was added. The temperature was slowly increased to 80° C. and the mixture was left with stirring overnight. The mixture was quenched with ice-cold water and extracted with EtOAc (2×150 mL). The combined organic layers were dried over Na₂SO₄ and evaporated to dryness. Flash chromatography (silica, EtOAc:petroleum ether 3:7) gave 1-(2-fluoro-ethyl)-1H-pyrazole-3-carboxylic acid ethyl ester as an off-white solid (7.5 g, 47%).

### Step 2:

To a solution of 1-(2-fluoro-ethyl)-1H-pyrazole-3-carboxylic acid ethyl ester (7.5 g, 40.30 mmol) in THF: CH₃OH:water (40:110:40 mL) was added LiOH (3.38 g, 80.61 mmol) at room temperature. The mixture was stirred for 4 h and then evaporated to dryness. Flash chromatography (silica, EtOAc:hexanes 4:1) gave the title compound IM36 as colorless crystals (2.7 g, 42%).

1H NMR (DMSO, 400 MHz) δ 12.66 (1H, s), 7.87-7.86 (1H, d, J=2.4 Hz), 6.71-6.70 (1H, d, J=2.0 Hz), 4.86-4.83 (1H, m), 4.74-4.72 (1H, m), 4.55-4.53 (1H, m), 4.48-4.46 (1H, m).

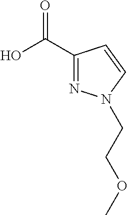

### IM37: 1-(2-Methoxy-ethyl)-1H-pyrazole-3-carboxylic acid

The compound was prepared as described for IM36 using 2-methoxy-1-bromoethane to give the title compound IM37 as a colorless solid.

1H NMR (DMSO, 400 MHz) δ 12.58 (1H, br, s), 7.79-7.78 (1H, d, J=2.4 Hz), 6.67-6.66 (1H, d, J=2.4 Hz), 4.34-4.31 (2H, m), 3.71-3.68 (2H, m), 3.22 (3H, s).

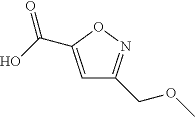

### IM38: 3-Methoxymethyl-isoxazole-5-carboxylic acid

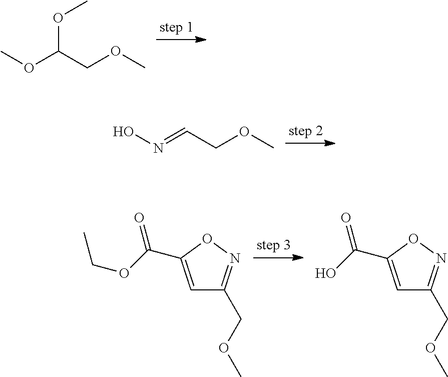

### Step 1:

To a solution of 1,1,2-trimethoxy-ethane (25.0 g, 208.07 mmol) in methanol (125 mL) was added NH₂OH.HCl (14.45 g, 208.07 mmol) at 0° C. The mixture was stirred for 16 h at room temperature. The mixture was cooled to 10° C. and aqueous NaOH solution (8.32 g, 208 mmol in 65 mL water) was added. The mixture was then stirred for 4 h at room temperature. The mixture was concentrated in vacuo and the remanence extracted with EtOAc (2×100 mL). The combined organic layers were dried over Na₂SO₄ and evaporated to dryness to give the crude oxime as a pale yellow liquid (12.0 g, 62%).

### Step 2:

To a solution of methoxy-acetaldehyde oxime (12.0 g, 134.69 mmol) in THF (100 mL) at 0° C. was added propynoic acid ethyl ester (26.40 g, 269.4 mmol) and 4% aqueous NaOCl solution (138 mL). The mixture was left without cooling overnight. The mixture was extracted with EtOAc (3×50 mL), the combined organic layers were dried over Na₂SO₄ and evaporated to dryness. Flash chromatography (silica, EtOAc:petroleum ether 1:9) gave 3-methoxymethylisoxazole-5-carboxylic acid ethyl ester as a orange oil (11.0 g, 30%).

### Step 3:

To a solution of 3-methoxymethyl-isoxazole-5-carboxylic acid ethyl ester (5.0 g, 27.0 mmol) in THF: CH₃OH: Water (20.0:7.5:7.5 mL) was added LiOH (2.27 g, 54.1 mmol) at 0° C. cooling was removed and the mixture was stirred at room temperature for 4 h. The volume was reduced in vacuo and pH was adjusted to 2-3 using 1N HCl. The mixture was evaporated to dryness. Flash chromatography (silica. MeOH:EtOAc 5:95) gave the title compound IM38 as a colorless solid (3.2 g, 76%).

1H NMR (DMSO, 400 MHz) δ 6.65 (1H, s), 4.45 (2H, s), 3.29 (3H, s).

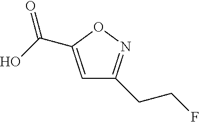

### IM39: 3-(2-Fluoro-ethyl)-isoxazole-5-carboxylic acid

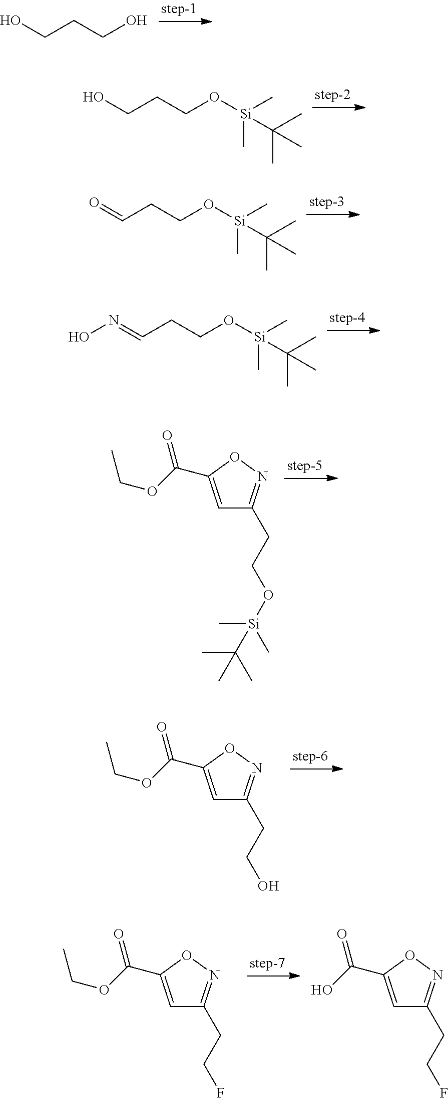

### Step 1:

To a suspension of NaH (7.57 g, 315.8 mmol) in THF kept at 0-10° C. was added propane1,3-diol (20.0 g, 263 mmol). The mixture was stirred for 30 Min. A solution of TBDMSCI (43.62 g, 289 mmol) in THF (100 mL) was added dropwise while keeping the temperature between 0-10° C. After stirring for additionally 30 min water was added and the resulting mixture was extracted with EtOAc (3×100 mL). The combined organic layers were dried over Na₂SO₄ and evaporated to dryness to give 3-(tert-butyl-dimethyl-silanyloxy)-propan-1-ol as a colorless liquid (48 g, 96%) which was used without further purification.

### Step 2:

To a solution of oxalyl chloride (7.34 g, 57.80 mmol) in methylene chloride cooled at −78° C. was added DMSO (9.03 g, 115.60 mmol). The mixture was stirred for 15 min. To this mixture was added 3-(tert-butyl-dimethyl-silanyloxy)-propan-1-ol (10.0 g, 52.54 mmol) followed by pyridine (8.3 g, 105.1 mmol). The mixture was stirred for 30 min at −78° C. Triethyl amine (26.53 g, 262.7 mmol) was then added and after 30 the mixture was quenched with water. The pH was adjusted to approx. 4 using 2N HCl and the mixture was extracted with methylene chloride (3×50 mL). The combined organic layers were washed with 10% aqueous K₂CO₃ solution (1×50 mL), dried over Na₂SO₄ and evaporated to dryness to give 3-(tertbutyl-dimethyl-silanyloxy)-propionaldehyde as a brown oil (9.0 g, 80%) which was used without further purification.

### Step 3:

To a solution of hydroxylamine hydrochloride (0.55 g, 7.97 mmol) in water at 20° C. was added NaOH (0.3 g, 7.97 mmol) in water (15 mL). The mixture was stirred for 10 min before addition of 3-(tert-butyl-dimethyl-silanyloxy)-propionaldehyde (1.0 g, 5.31 mmol). The mixture was stirred for 16 h at room temperature and then extracted with EtOAc (3×20 mL). The combined organic layers were dried over Na₂SO₄ and evaporated to dryness to give 3-(tertbutyl-dimethyl-silanyloxy)-propionaldehyde oxime as a yellow liquid (0.7 g, 70%) which was used without further purification.

### Step 4:

To a solution of 3-(tert-butyl-dimethyl-silanyloxy)-propionaldehyde oxime (12.0 g, 59.0 mmol) in THF (102 mL) at 20° C. was added propynoic acid ethyl ester (11.56 g, 118.0 mmol) and 4% aqueous NaOCl solution (205 mL). The mixture was stirred for 4 h at room temperature and then extracted with EtOAc (3×100 mL). The combined organic layers were dried over anhydrous Na₂SO₄ and evaporated to dryness. Flash chromatography (silica, EtOAc:petroleum ether 5:95) gave 3-[2-(tert-butyl-dimethyl-silanyloxy)-ethyl]-isoxazole-5-carboxylic acid ethyl ester as a pale yellow liquid (6.0 g, 35%).

### Step 5:

To a solution of 3-(2-(tert-butyl-dimethyl-silanyloxy)-ethyl)-isoxazole-5-carboxylic acid ethyl ester (70.0 g, 234.1 mmol) in THF (600 mL) at 10° C. was added TBAF (1M in THF, 351.2 mL, 351.2 mmol) and the mixture was stirred at room temperature for 1 h. The mixture was quenched with water and subsequently extracted with EtOAc (3×200 mL). The combined organic layers were dried over Na₂SO₄ and evaporated to dryness. Flash chromatography (silica, EtOAc:petroleum ether 1:4) gave 3-(2-hydroxy-ethyl)-isoxazole-5-carboxylic acid ethyl ester as a yellow liquid (15.0 g, 23%).

### Step 6:

To a solution of 3-(2-hydroxy-ethyl)-isoxazole-5-carboxylic acid ethyl ester (8.0 g, 43.24 mmol) in methylene chloride (50 mL) at 0° C. was added Deoxo-fluor (14.35 g, 64.9 mmol). The mixture was stirred at room temperature for 16 h. The mixture was quenched with saturated, aqueous NaHCO₃ solution (100 mL) and extracted with methylene chloride (3×50 mL). The combined organic layers were dried over Na₂SO₄ and evaporated to dryness. Flash chromatography (silica, EtOAc:petroleum ether 2:98) gave 3-(2-fluoro-ethyl)-isoxazole-5-carboxylic acid ethyl ester as a yellow liquid (1.72 g, 41%).

### Step 7:

To a solution of 3-(2-fluoro-ethyl)-isoxazole-5-carboxylic acid ethyl ester (3.0 g, 16.04 mmol) in THF: CH₃OH: Water (13.5:4.5:4.5 mL) was added LiOH (1.34 g, 32.1 mmol) at 10° C. The mixture was stirred at room temperature for 2 h and then the mixture was concentrated in vacuo. The pH was adjusted to 2-3 using 1N HCl and the mixture was evaporated to dryness. Flash chromatography (silica, MeOH:EtOAc 1:9) gave the title compound IM39 as a colorless solid (2.3 g, 75%).

1H NMR (DMSO, 400 MHz) δ 6.77 (1H, s), 4.77-4.74 (1H, m), 4.65-4.62 (1H, m), 3.03-3.01 (1H, m), 2.97-2.94 (1H, m).

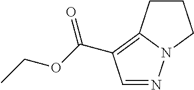

### IM40: 5,6-Dihydro-4H-pyrrolo[1,2-b]pyrazole-3-carboxylic acid ethyl ester

The synthesis is described in WO2004104006A2.

1H NMR (DMSO, 400 MHz) δ 7.80 (1H, s), 4.20-4.15 (2H, m), 4.12-4.09 (2H, m), 2.99-2.96 (2H, m), 2.61-2.53 (2H, m), 1.26-1.23 (3H, t, J=7.0 Hz).

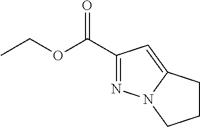

### IM41: 5,6-Dihydro-4H-pyrrolo[1,2-b]pyrazole-2-carboxylic acid ethyl ester

The synthesis is described in WO2004104006A2.

1H NMR (DMSO, 400 MHz) δ 6.46 (1H, s), 4.26-4.20 (2H, m), 4.14-4.10 (2H, m), 2.87-2.83 (2H, m), 2.58-2.50 (2H, m), 1.28-1.24 (3H, t, J=7.2 Hz).

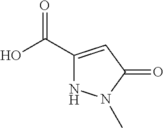

### IM42: 1-Methyl-5-oxo-2,5-dihydro-1H-pyrazole-3-carboxylic acid

Prepared by hydrolysis of the commercially available ethyl 5-hydroxy-1-methyl-1H-pyrazole-3-carboxylate.

### Example 1

### Compounds of the Invention

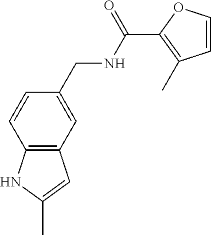

### 1: 3-methyl-N-[(2-methyl-1H-indol-5-yl)methyl]furan-2-carboxamide

Compound IM1 (0.06 mmol) was dissolved in DMF (240 uL). 3-Methyl-furan-2-carboxylic acid (0.062 mmol), N,N-Diisopropylethylamine (54 uL, 0.31 mmol) and N,N,N′,N′-tetramethyl-O-(7-azabenzotriazol-1-yl)uronium hexafluorophosphate (24 mg, 0.062 mmol) were added. The reaction mixture was shaken at room temperature overnight. The reaction mixture was purified by preparative LC-MS (Method I) to give the title compound.

LC-MS (m/z) 269 (MH⁺); t_{R}=1.34. (Method A).

The following compounds were prepared analogously:

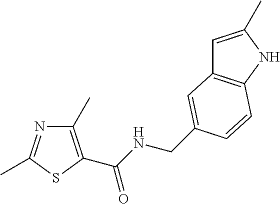

### 2: 2,4-dimethyl-N-[(2-methyl-1H-indol-5-311)methyl]thiazole-5-carboxamide

Prepared using IM1 and 2,4-dimethyl-thiazole-5-carboxylic acid.

LC-MS (m/z) 300 (MH⁺); t_{R}=1.19 (Method A).

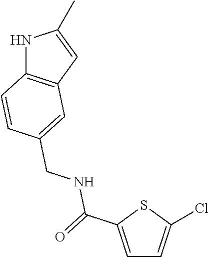

### 3: 5-Chloro-thiophene-2-carboxylic acid (2-methyl-1H-indol-5-ylmethyl)-amide

Prepared using IM1 and 5-chloro-thiophene-2-carboxylic acid.

LC-MS (m/z) 305 (MH⁺); t_{R}=1.52 (Method A).

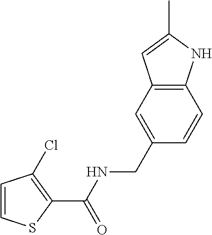

### 4: 3-chloro-N-[(2-methyl-1H-indol-5-yl)methyl]thiophene-2-carboxamide

Prepared using IM1 and 3-chloro-thiophene-2-carboxylic acid.

LC-MS (m/z) 305 (MN); t_{R}=1.49 (Method A).

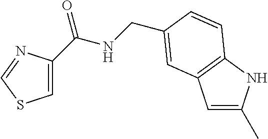

### 5: N-[(2-methyl-1H-indol-5-yl)methyl]thiazole-4-carboxamide

Prepared using IM1 and thiazole-4-carboxylic acid.

LC-MS (m/z) 272 (MH⁺); t_{R}=1.49 (Method A).

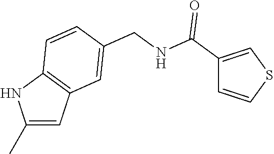

### 6: N-[(2-methyl-1H-indol-5-yl)methyl]thiophene-3-carboxamide

Prepared using IM1 and thiophene-3-carboxylic acid.

LC-MS (m/z) 271 (MH⁺); t_{R}=1.19 (Method A).

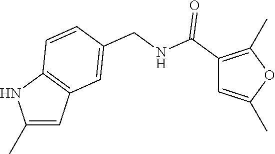

### 7: 2,5-dimethyl-N-[(2-methyl-1H-indol-5-yl)methy]furan-3-carboxamide

Prepared using IM1 and 2,5-dimethyl-furan-3-carboxylic acid.

LC-MS (m/z) 283 (MH⁺); t_{R}=1.44 (Method A).

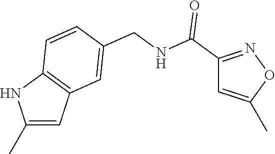

### 8: 5-methyl-N-[(2-methyl-1H-indol-5-yl)methyl]isoxazole-3-carboxamide

Prepared using IM1 and 5-methyl-isoxazole-3-carboxylic acid.

LC-MS (m′ z) 270 (MH⁺); t_{R}=1.22 (Method A).

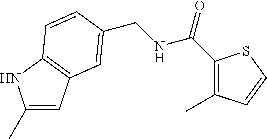

### 9: 3-methyl-N-[(2-methyl-1H-indol-5-yl)methyl]thiophene-2-carboxamide

Prepared using IM1 and 3-methyl-thiophene-2-carboxylic acid.

LC-MS (m/z) 285 (MH⁺); t_{R}=1.37 (Method A).

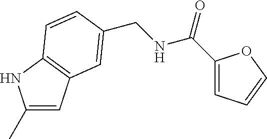

### 10: N-[(2-methyl-1H-indol-5-yl)methyl]furan-2-carboxamide

Prepared using IM1 and furan-2-carboxylic acid.

LC-MS (m/z) 255 (MH⁺); t_{R}=1.13 (Method A).

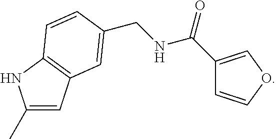

### 11: N-[(2-methyl-1H-indol-5-yl)methyl]furan-3-carboxamide

Prepared using IM1 and furan-3-carboxylic acid.

LC-MS (m/z) 255 (MH⁺); t_{R}=1.09 (Method A).

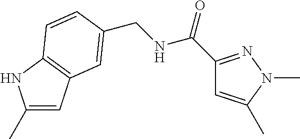

### 12: 1,5-dimethyl-N-[(2-methyl-1H-indol-5-yl)methyl]pyrazole-3-carboxamide

Prepared using IM1 and 1,5-dimethyl-1H-pyrazole-3-carboxylic acid.

LC-MS (m/z) 283 (MH⁺); t_{R}=1.18 (Method A).

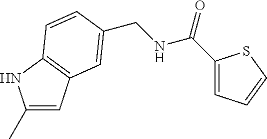

### 13: N-[(2-methyl-1H-indol-5-yl)methyl]thiophene-2-carboxamide

Prepared using IM1 and thiophene-2-carboxylic acid.

LC-MS (m/z) 271 (MH⁺); t_{R}=1.19 (Method A).

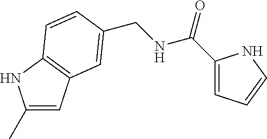

### 14: N-[(2-methyl-1H-indol-5-yl)methyl]-1H-pyrrole-2-carboxamide

Prepared using IM1 and 1H-pyrrole-2-carboxylic acid.

LC-MS (m/z) 254 (MH⁺); tᵣ=1.10 (Method A).

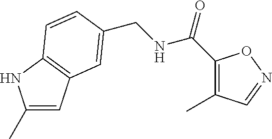

### 15: 4-methyl-N-[(2-methyl-1H-indol-5-yl)methyl]isoxazole-5-carboxamide

Prepared using IM1 and 4-methyl-isoxazole-5-carboxylic acid.

LC-MS (m/z) 270 (MH⁺); t_{R}=1.24 (Method A).

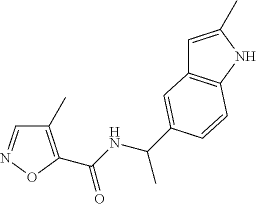

### 16: 4-methyl-N-[1-(2-methyl-1H-indol-5-yl)ethyl]isoxazole-5-carboxamide

Prepared using IM2 and 4-methyl-isoxazole-5-carboxylic acid.

LC-MS (m/z) 284 (MH⁺); t_{R}=1.32 (Method A).

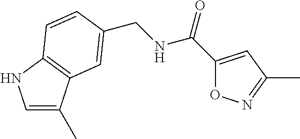

### 17: 3-methyl-N-[(3-methyl-1H-indol-5-34)methyl]isoxazole-5-carboxamide

Prepared using IM3 and 3-methyl-isoxazole-5-carboxylic acid.

LC-MS (m/z) 270 (MH⁺); t_{R}=1.21 (Method A).

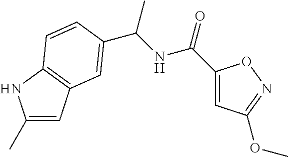

### 18: 3-methoxy-N-[1-(2-methyl-1H-indol-5-yl)ethyl]isoxazole-5-carboxamide

Prepared using IM2 and 3-methoxy-Isoxazole-5-carboxylic acid.

LC-MS (m/z) 300 (MH⁺); t_{R}=1.33 (Method A).

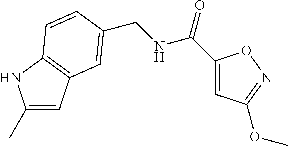

### 19: 3-methoxy-N-[(2-methyl-1H-indol-5-yl)methyl]isoxazole-5-carboxamide

Prepared using IM1 and 3-methoxy-isoxazole-5-carboxylic acid.

LC-MS (m/z) 286 (MH⁺); t_{R}=1.22 (Method A).

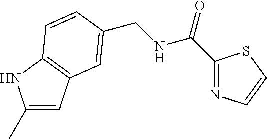

### 20: N-[(2-methyl-1H-indol-5-yl)methyl]thiazole-2-carboxamide

Prepared using IM1 and thiazole-2-carboxylic acid.

LC-MS (m/z) 272 (MH⁺); t_{R}=1.26 (Method A).

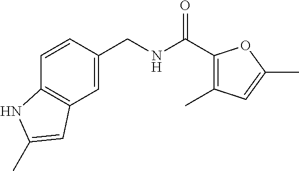

### 21: 2,4-dimethyl-N-[(2-methyl-1H-indol-5-yl)methyl]oxazole-5-carboxamide

Prepared using IM1 and 2,4-dimethyl-oxazole-5-carboxylic acid.

LC-MS (m/z) 284 (MH⁺); t_{R}=0.55 (Method B).

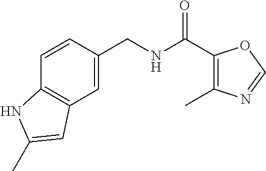

### 22: 4-methyl-N-[(2-methyl-1H-indol-5-yl)methyl]oxazole-5-carboxamide

Prepared using IM1 and 4-methyl-oxazole-5-carboxylic acid.

LC-MS (m/z) 270 (MH⁺); t_{R}=0.52 (Method B).

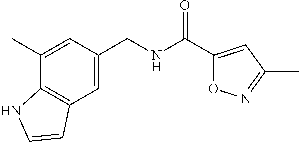

### 23: 3-methyl-N-[(7-methyl-1H-indol-5-yl)methyl]isoxazole-5-carboxamide

Prepared using IM4 and 3-methyl-isoxazole-5-carboxylic acid.

LC-MS (m/z) 271 (MH⁺); t_{R}=0.55 (Method B).

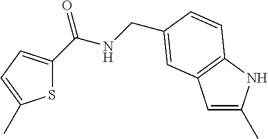

### 24: 5-methyl-N-[(2-methyl-1H-indol-5-yl)methyl]thiophene-2-carboxamide

A round bottomed flask was charged with IM1 (1.00 g, 6.24 mmol), 5-methyl-2-thiophenecarboxylic acid (0.976 g, 6.86 mmol), N,N,N′,N′-tetramethyl-O-(7-azabenzotriazol-1-yl)uronium hexafluorophosphate (2.61 g, 6.86 mmol) and triethylamine (1.30 ml, 9.36 mmol) in N,N-dimethylformamide (25.0 mL, 323 mmol). The suspension was stirred at room temperature overnight. To the reaction mixture was added saturated aqueous NaHCO₃ solution until pH reached 8-10. The mixture was extracted with EtOAc (3×100 mL). The combined organic layers were washed with brine (3×70 ml), dried over MgSO₄, filtered and evaporated to dryness. Flash chromatography (silica, EtOAc/heptane 1:1) gave the target molecule as a colorless solid (0.790 g, 44%).

LC-MS (m/z) 285 (MH⁺); t_{R}=1.37. (Method A).

The following compounds were prepared analogously:

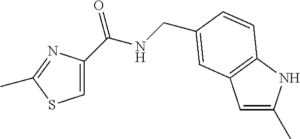

### 25: 2-methyl-N-[(2-methyl-1H-indol-5-yl)methyl]thiazole-4-carboxamide

Prepared using IM1 and 2-methyl-thiazole-4-carboxylic acid.

LC-MS (m/z) 287 (MH⁺); t_{R}=1.36 (Method A).

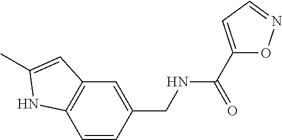

### 26: N-[(2-methyl-1H-indol-5-yl)methyl]isoxazole-5-carboxamide

Prepared using IM1 and isoxazole-5-carboxylic acid.

LC-MS (m/z) 256 (MH⁺); t_{R}=1.03 (Method A).

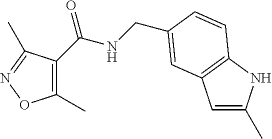

### 27: 3,5-dimethyl-N-[(2-methyl-1H-indol-5-yl)methyl]isoxazole-4-carboxamide

Prepared using IM1 and 3,5-dimethyl-isoxazole-4-carboxylic acid.

LC-MS (m/z) 284 (MH⁺); t_{R}=1.15 (Method A).

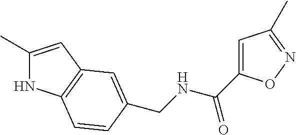

### 28: 3-methyl-N-[(2-methyl-1H-indol-5-yl)methyl]isoxazole-5-carboxamide

Prepared using IM1 and 3-methyl-isoxazole-5-carboxylic acid.

LC-MS (m/z) 270 (MH⁺); t_{R}=1.17 (Method A).

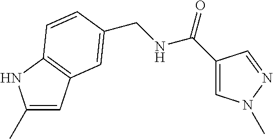

### 29: 1-methyl-N-[(2-methyl-1H-indol-5-yl)methyl]pyrazole-4-carboxamide

Prepared using IM1 and 1-methyl-1H-pyrazole-4-carboxylic acid.

LC-MS (m/z) 269 (MH⁺); t_{R}=0.95 (Method A).

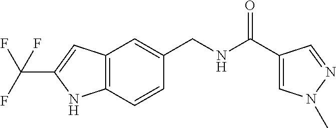

### 30: 1-methyl-N-[[2-(trifluoromethyl)-1H-indol-5-yl]methyl]pyrazole-4-carboxamide

Prepared using IM5 and 1-methyl-1H-pyrazole-4-carboxylic acid.

LC-MS (m/z) 323 (MH⁺); t_{R}=1.30 (Method A).

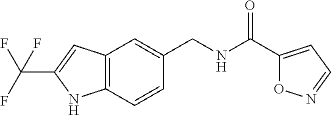

### 31: N-[[2-(trifluoromethyl)-1H-indol-5-yl]methyl]isoxazole-5-carboxamide

Prepared using IM5 and isoxazole-5-carboxylic acid.

LC-MS (m/z) 310 (MH⁺); t_{R}=1.38 (Method A).

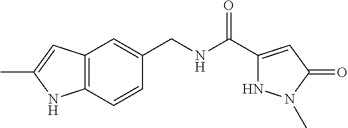

### 32: 2-methyl-N-[(2-methyl-1H-indol-5-yl)methyl]-3-oxo-1H-pyrazole-5-carboxamide

Prepared using IM1 and IM42.

LC-MS (m/z) 285 (MH⁺); t_{R}=1.03 (Method A).

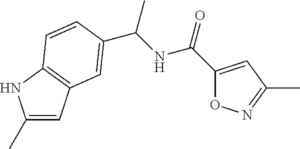

### 33: 3-methyl-N-[1-(2-methyl-1H-indol-5-yl)ethyl]isoxazole-5-carboxamide

Prepared using IM2 and 3-methyl-isoxazole-5-carboxylic acid.

LC-MS (m/z) 284 (MH⁺); t_{R}=1.24 (Method A).

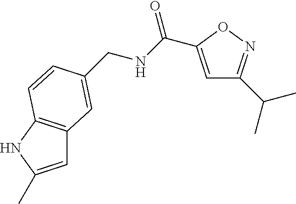

### 34: 3-isopropyl-N-[(2-methyl-1H-indol-5-yl)methyl]isoxazole-5-carboxamide

Prepared using IM1 and 3-isopropyl-isoxazole-5-carboxylic acid.

LC-MS (m/z) 298 (MH⁺); t_{R}=1.44 (Method A).

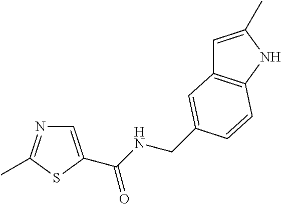

35: 2-methyl-N-[(2-methyl-1H-indol-5-yl)methyl]thiazole-5-carboxamide

Prepared using IM1 and 2-methyl-thiazole-5-carboxylic acid.

LC-MS (m/z) 286 (MH⁺): t_{R}=1.17 (Method A).

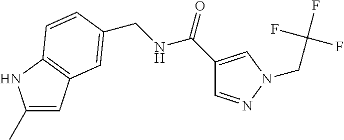

36: N-[(2-methyl-1H-indol-5-yl)methyl]-1-(2,2,2-trifluoroethyl)pyrazole-4-carboxamide

Prepared using IM1 and IM29.

LC-MS (m/z) 337 (MH⁺); t_{R}=1.24 (Method A).

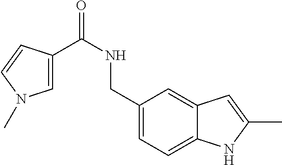

### 37: 1-methyl-N-[(2-methyl-1H-indol-5-yl)methyl]pyrrole-3-carboxamide

Prepared using IM1 and 1-methyl-1H-pyrrole-3-carboxylic acid.

LC-MS (m/z) 268 (MH⁺); t_{R}=1.09 (Method A).

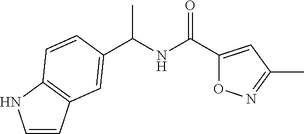

### 38: N-[1-(1H-indol-5-yl)ethyl]-3-methyl-isoxazole-5-carboxamide

Prepared using IM6 and 3-methyl-isoxazole-5-carboxylic acid.

LC-MS (m/z) 144 (MH⁺); t_{R}=1.12 (Method A).

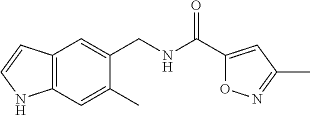

### 39: 3-methyl-N-[(6-methyl-1H-indol-5-yl)methyl]isoxazole-5-carboxamide

Prepared using IM7 and 3-methyl-isoxazole-5-carboxylic acid.

LC-MS (m/z) 270 (MH⁺); t_{R}=1.09 (Method A).

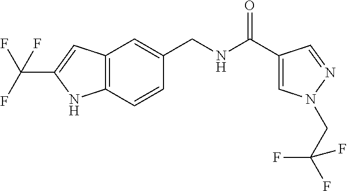

### 40: 1-(2,2,2-trifluoroethyl)-N-[[2-(trifluoromethyl)-1H-indol-5-yl]methyl]pyrazole-4-carboxamide

Prepared using IM5 and IM29.

LC-MS (m/z) 391 (MH⁺); t_{R}=1.48 (Method A).

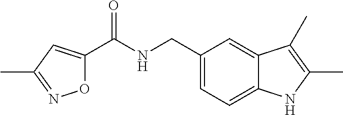

### 41: N-[(2,3-dimethyl-1H-indol-5-yl)methyl]-3-methyl-isoxazole-5-carboxamide

Prepared using IM8 and 3-methyl-isoxazole-5-carboxylic acid.

LC-MS (m/z) 284 (MH⁺); t_{R}=1.28 (Method A).

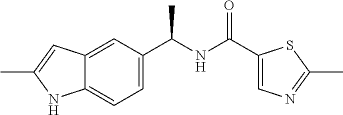

### 42: 2-methyl-N-[(1R)-1-(2-methyl-1H-indol-5-yl)ethyl]thiazole-5-carboxamide

Prepared using IM9 and 2-methyl-thiazole-5-carboxylic acid.

LC-MS (m/z) 300 (MH⁺); t_{R}=1.18 (Method A).

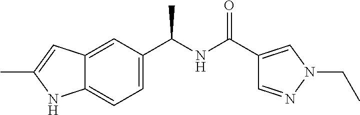

### 43: 1-ethyl-N-[(1R)-1-(2-methyl-1H-indol-5-yl)ethyl]pyrazole-4-carboxamide

Prepared using IM9 and 1-ethyl-1H-pyrazole-4-carboxylic acid.

LC-MS (m/z) 298 (MH⁺); t_{R}=1.14 (Method A).

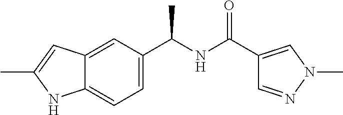

### 44: 1-methyl-N-[(1R)-1-(2-methyl-1H-indol-5-yl)ethyl]pyrazole-4-carboxamide

Prepared using IM9 and 1-methyl-1H-pyrazole-4-carboxylic acid.

LC-MS (m/z) 283 (MH⁺); t_{R}=1.04 (Method A).

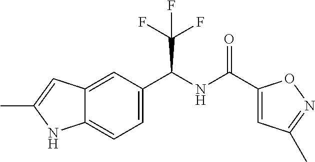

### 45: 3-methyl-N-[(1S)-2,2,2-trifluoro-1-(2-methyl-1H-indol-5-yl)ethyl]isoxazole-5-carboxamide

Prepared using IM10 and 3-methyl-isoxazole-5-carboxylic acid.

LC-MS (m/z) 339 (MH⁺); t_{R}=1.07 (Method A).

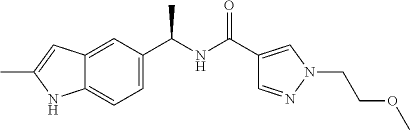

### 46: 1(2-methoxyethyl)-N-[(1R)-1-(2-methyl-1H-indol-5-yl)ethyl]pyrazole-4-carboxamide

Prepared using IM9 and IM30.

LC-MS (m/z) 327 (MN⁺); t_{R}=1.17 (Method A).

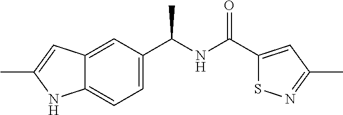

### 47: 3-methyl-N-[(1R)-1-(2-methyl-1H-indol-5-yl)ethyl]isothiazole-5-carboxamide

Prepared using IM9 and 3-methyl-isothiazole-5-carboxylic acid.

LC-MS (m/z) 300 (MH⁺); t_{R}=1.14 (Method A).

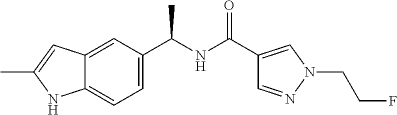

### 48: 1-(2-fluoroethyl)-N-[(1R)-1-(2-methyl-1H-indol-5-yl)ethyl]pyrazole-4-carboxamide

Prepared using IM9 and IM31.

LC-MS (m/z) 315 (MH⁺); t_{R}=0.53 (Method C).

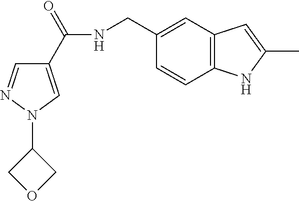

### 49: N-[(2-methyl-1H-indol-5-yl)methyl]-1-(oxetan-3-yl)pyrazole-4-carboxamide

Prepared using IM1 and IM32.

LC-MS (m/z) 311 (MH⁺); t_{R}=0.46 (Method C).

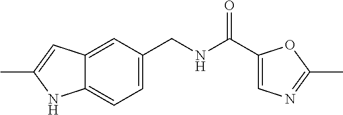

### 50: 2-methyl-N-[(2-methyl-1H-indol-5-yl)methyl]oxazole-5-carboxamide

Prepared using IM1 and 2-methyl-oxazole-5-carboxylic acid.

LC-MS (m/z) 270 (MH⁺); t_{R}=0.49 (Method C).

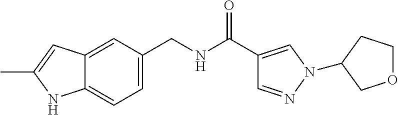

### 51: N-[(2-methyl-1H-indol-5-yl)methyl]-1-tetrahydrofuran-3-yl-pyrazole-4-carboxamide

Prepared using IM1 and IM33.

LC-MS (m/z) 325 (MH⁺); t_{R}=0.48 (Method C).

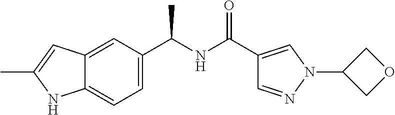

### 52: N-[(1R)-1-(2-methyl-1H-indol-5-yl)ethyl]-1-(oxetan-3-yl)pyrazole-4-carboxamide

Prepared using IM9 and IM32.

1H NMR (500 MHz, DMSO) δ 10.80 (s, 1H), 8.43-8.26 (m, 2H), 8.04 (s, 1H), 7.37 (s, 1H), 7.19 (d, 1H), 7.01 (d, 1H), 6.07 (s, 1H), 5.66-5.55 (m, 1H), 5.28-5.12 (m, 1H), 4.89 (m, 4H), 2.36 (s, 3H), 1.48 (d, 3H).

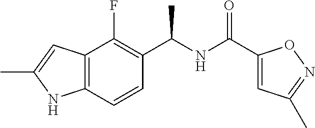

### 53: N-[(1R)-1-(4-fluoro-2-methyl-1H-indol-5-yl)ethyl]-3-methyl-isoxazole-5-carboxamide

Prepared using IM11 and 3-methyl-isoxazole-5-carboxylic acid. The final compound was purified by preparative HPLC (Method G) followed by preparative SFC (Method K).

1HNMR (CDCl₃, 400 MHz): b 7.99 (s, 1H), 7.05-6.99 (m, 2H), 6.71 (s, 1H), 6.30 (s, 1H), 5.55-5.47 (m, 1H), 2.44 (s, 3H), 2.34 (s, 3H), 1.60 (d, 3H).

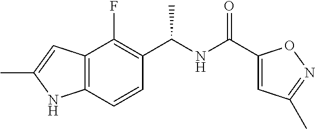

### 54: N-[(1S)-1-(4-fluoro-2-methyl-1H-indol-5-yl)ethyl]-3-methyl-isoxazole-5-carboxamide

Prepared using IM11 and 3-methyl-isoxazole-5-carboxylic acid. The final compound was purified by preparative HPLC (Method G) followed by preparative SFC (Method K).

1HNMR (CDCl₃, 400 MHz): δ 8.07 (s, 1H), 7.05-6.99 (m, 2H), 6.71 (s, 1H), 6.29 (s, 1H), 5.55-5.48 (m, 1H), 2.44 (s, 3H), 2.34 (s, 3H), 1.66 (d, 3H).

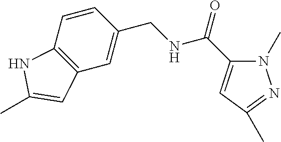

### 55: 2,5-dimethyl-N-[(2-methyl-1H-indol-5-yl)methyl]pyrazole-3-carboxamide

Prepared using IM1 and 2,5-dimethyl-2H-pyrazole-3-carboxylic acid.

LC-MS (m/z) 283 (MH⁺); t_{R}=1.22 (Method).

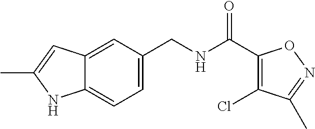

### 56: 4-chloro-3-methyl-N-[(2-methyl-1H-indol-5-yl)methyl]isoxazole-5-carboxamide

Prepared using IM1 and IM34.

1H NMR (CDCl₃ 400 MHz): δ 7.96 (br s, 1H), 7.47 (s, 1H), 7.25-7.27 (m, 1H), 7.08 (m, 1H), 6.72 (br s, 1H), 6.19 (s, 1H), 4.67 (d, 2H), 2.44 (s, 3H), 2.23 (s, 3H).

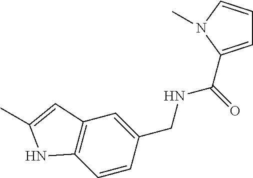

### 57: 1-methyl-N-[(2-methyl-1H-indol-5-yl)methyl]pyrrole-2-carboxamide

1-Methyl-1H-pyrrole-2-carboxylic acid (0.100 g, 0.799 mmol) and 1-hydroxybenzotriazole (0.119 g, 0.879 mmol) was dissolved in N,N-Dimethylformamide (5 mL, 60 mmol). N-(3-Dimethylaminopropyl)-N′-ethylcarbodiimide hydrochloride (0.168 g, 0.879 mmol) was added and the mixture stirred at room temperature for 2 hours. Compound IM1 (0.141 g, 0.879 mmol) and triethylamine (0.167 mL, 1.20 mmol) was added and the mixture was stirred at room temperature overnight. The mixture was quenched with water and extracted with EtOAc. The combined organic layers were washed with brine and dried over Na₂SO₄ and evaporated to dryness. Flash chromatography (silica, EtOAc:heptanes 1:1) gave the title compound (149 mg, 70%).

LC-MS (m/z) 268 (MH⁺); t_{R}=1.29 (Method A).

The following compounds were prepared analogously:

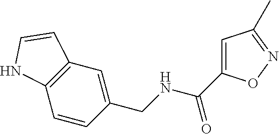

### 58: N-(1H-indol-5-ylmethyl)-3-methyl-isoxazole-5-carboxamide

Prepared using IM12 and 3-methyl-isoxazole-5-carboxylic acid.

LC-MS (m/z) 256 (MH⁺); t_{R}=0.98 (Method A).

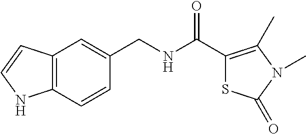

### 59: N-(1H-indol-5-ylmethyl)-3,4-dimethyl-2-oxo-thiazole-5-carboxamide

Prepared using IM12 and 3,4-dimethyl-2-oxo-2,3-dihydro-thiazole-5-carboxylic acid.

LC-MS (m/z) 302 (MH⁺); t_{R}=0.97 (Method A).

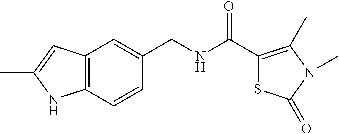

### 60: 3,4-dimethyl-N-[(2-methyl-1H-indol-5-yl)methyl]-2-oxo-thiazole-5-carboxamide

Prepared using IM1 and 3,4-dimethyl-2-oxo-2,3-dihydro-thiazole-5-carboxylic acid.

LC-MS (m/z) 316 (MH⁺); t_{R}=1.10 (Method A).

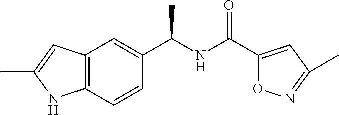

### 61: 3-methyl-N-[(1R)-1-(2-methyl-1H-indol-5-yl)ethyl]isoxazole-5-carboxamide

Prepared using IM9 and 3-methyl-isoxazole-5-carboxylic acid.

LC-MS (m/z) 284 (MH⁺); t_{R}=1.25 (Method A).

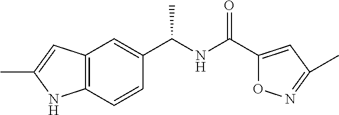

### 62: 3-methyl-N-[(1S)-1-(2-methyl-1H-indol-5-yl)ethyl]isoxazole-5-carboxamide

Prepared using IM13 and 3-methyl-isoxazole-5-carboxylic acid.

LC-MS (m/z) 284 (MH⁺); t_{R}=1.25 (Method A).

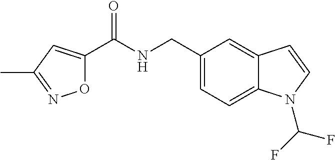

### 63: N-[[1-(difluoromethyl)indol-5-yl]methyl]-3-methyl-isoxazole-5-carboxamide

Prepared using IM14 and 3-methyl-isoxazole-5-carboxylic acid.

LC-MS (m/z) 306 (MH⁺); t_{R}=1.29 (Method A).

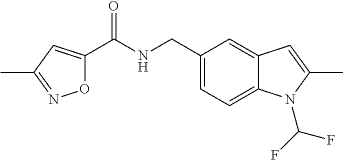

### 64: N-[[1-(difluoromethyl-2-methyl-indol-5-yl]methyl]-3-methyl-isoxazole-5-carboxamide

Prepared using IM15 and 3-methyl-isoxazole-5-carboxylic acid.

1H NMR (DMSO, 400 MHz) δ 9.43-9.40 (1H, m), 8.07-7.77 (1H, t, J=58.4 Hz), 7.58-7.56 (1H, d), 7.42 (1H, s), 7.17-7.14 (1H, d), 6.93 (1H, s), 6.39 (1H, s), 4.50-4.49 (2H, m), 2.47 (3H, s), 2.29 (3H, s).

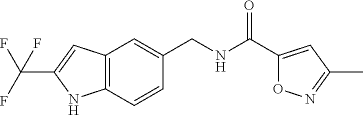

### 65: 3-methyl-N-[[2-(trifluromethyl)-1H-indol-5-yl]methyl]isoxazole-5-carboxamide

Prepared using IM5 and 3-methyl-isoxazole-5-carboxylic acid.

LC-MS (m/z) 324 (MH⁺); t_{R}=1.43 (Method A).

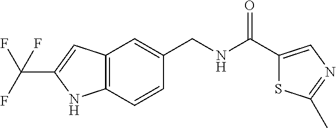

### 66: 2-methyl-N-[[2-(trifluoromethyl)-1H-indol-5-yl]methyl]thiazole-5-carboxamide

Prepared using IM5 and 2-methyl-thiazole-5-carboxylic acid.

LC-MS (m/z) 340 (MH⁺); t_{R}=1.46 (Method A).

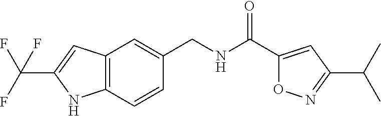

### 67: 3-isopropyl-N-[[2-(trifluoromethyl)-1H-indol-5-yl]methyl]isoxazole-5-carboxamide

Prepared using IM5 and 3-isopropyl-isoxazole-5-carboxylic acid.

LC-MS (m/z) 352 (MN⁺); t_{R}=1.74 (Method A).

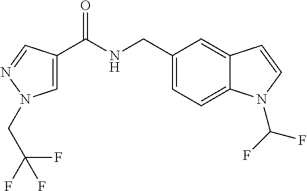

### 68: N-[[1-(difluoromethyl)indol-5-yl]methyl]-1-(2,2,2-trifluoroethyl)pyrazole-4-carboxamide

Prepared using IM14 and IM29.

LC-MS (m/z) 373 (MR⁺); t_{R}=1.34 (Method A).

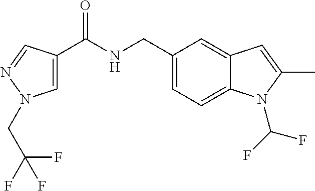

### 69: N-[[1-(difluoromethyl)-2-methyl-indol-5-yl]methyl]-1-(2,2,2-trifluoroethyl)pyrazole-4-carboxamide

Prepared using IM15 and IM29.

LC-MS (m/z) 387 (MH⁺); t_{R}=1.45 (Method A).

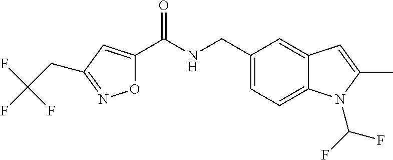

### 70: N-[[1-(difluoromethyl)-2-methyl-indol-5-yl]methyl]-3-(2,2,2-trifluoroethyl)isoxazole-5-carboxamide

Prepared using IM15 and IM35.

1H NMR (DMSO, 400 MHz) δ 9.55-9.52 (1H, m), 8.07-7.78 (1H, t, J=58.4 Hz), 7.59-7.57 (1H, d), 7.44 (1H, s), 7.18-7.16 (1H, m), 7.14 (1H, s), 6.40 (1H, s), 4.51-4.50 (2H, m), 4.04-3.95 (2H, m), 2.47 (3H, s).

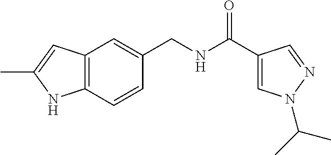

### 71: 1-isopropyl-N-[(2-methyl-1H-indol-5-yl)methyl]pyrazole-4-carboxamide

Prepared using IM1 and 1-isopropyl-1H-pyrazole-4-carboxylic acid.

LC-MS (m/z) 297 (MH⁺); t_{R}=1.16 (Method A).

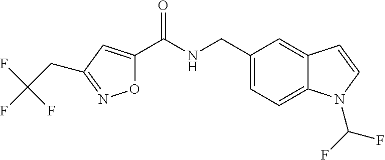

### 72: N-[[1-(difluoromethyl)indol-5-yl]methyl]-3-(2,2,2-trifluoroethyl)isoxazole-5-carboxamide

Prepared using IM14 and IM35.

1H NMR (DMSO, 400 MHz) δ 9.58-9.55 (1H, m), 8.11-7.81 (1H, t, J=59.6 Hz), 7.65-7.58 (3H, m), 7.27-7.25 (1H, d), 7.14 (1H, s), 6.68-6.67 (1H, m), 4.54-4.53 (2H, m), 4.04-3.95 (2H, m).

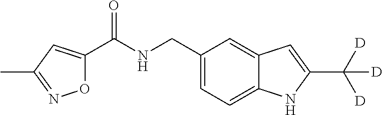

### 73: 3-methyl-N-[[2-(trideuteriomethyl)-1H-indol-5-yl]methyl]isoxazole-5-carboxamide

Prepared using IM16 and 3-methyl-isoxazole-5-carboxylic acid.

LC-MS (m/z) 273 (MH⁺); t_{R}=1.16 (Method A).

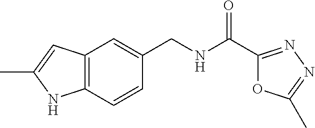

### 74: 5-methyl-N-[(2-methyl-1H-indol-5-yl)methyl]-1,3,4-oxadiazole-2-carboxamide

Prepared using IM1 and 5-methyl-[1,3,4]oxadiazole-2-carboxylic acid.

LC-MS (m/z) 271 (MH⁺); t_{R}=0.97 (Method A).

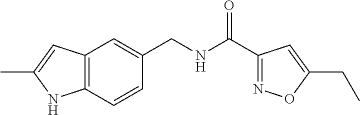

### 75: 5-ethyl-N-[(2-methyl-1H-indol-5-yl)methyl]isoxazole-3-carboxamide

Prepared using IM1 and 5-ethyl-isoxazole-3-carboxylic acid.

LC-MS (m/z) 284 (MH⁺); t_{R}=1.38 (Method A).

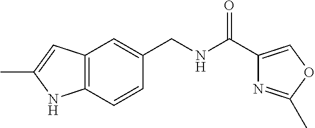

### 76: 2-methyl-N-[(2-methyl-1H-indol-5-yl)methyl]oxazole-4-carboxamide

Prepared using IM1 and 2-methyl-oxazole-4-carboxylic acid.

LC-MS (m/z) 270 (MH⁺); t_{R}=1.16 (Method A).

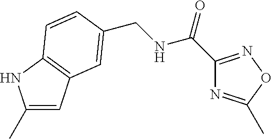

### 77: 5-methyl-N-[(2-methyl-1H-indol-5-yl)methyl]-1,2,4-oxadiazole-3-carboxamide

Prepared using IM1 and 5-methyl-[1,2,4]oxadiazole-3-carboxylic acid.

LC-MS (m/z) 271 (MH⁺); t_{R}=1.02 (Method A).

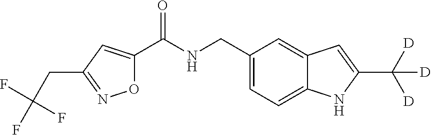

### 78: N-[[2-(trideuteriomethyl)-1H-indol-5-yl]methyl]-3-(2,2,2-trifluoroethyl)isoxazole-5-carboxamide

Prepared using IM16 and IM35.

LC-MS (m/z) 341 (MH⁺); t_{R}=1.35 (Method A).

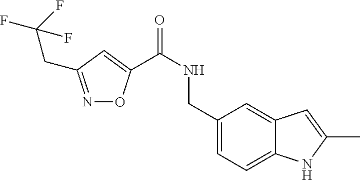

### 79: N-[2-methyl-1H-indol-5-yl)methyl]-3-(2,2,2-trifluoroethyl)isoxazole-5-carboxamide

Prepared using IM1 and IM36.

LC-MS (m/z) 338 (MH⁺); t_{R}=1.35 (Method A).

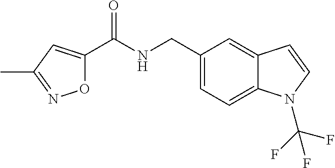

### 80: 3-methyl-N-[[1-(trifluoromethyl)indol-5-yl]methyl]isoxazole-5-carboxamide

Prepared using IM17 and 3-methyl-isoxazole-5-carboxylic acid.

LC-MS (m/z) 324 (MH⁺); t_{R}=1.57 (Method A).

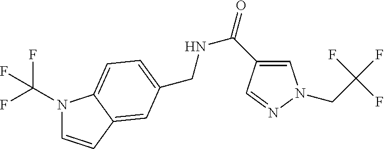

### 81: 1-(2,2,2-trifluoroethyl)-N-[[1-(trifluoromethyl)indol-5-yl]methyl]pyrazole-4-carboxamide

Prepared using IM17 and IM29.

LC-MS (m/z) 391 (MH⁺); t_{R}=1.61 (Method A).

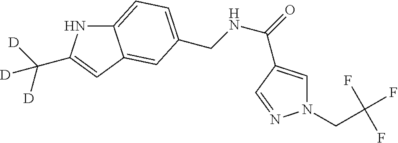

### 82: N-[[2-(trideuteriomethyl-1H-indol-5-yl]methyl]-1-(2,2,2-trifluoroethyl)pyrazole-4-carboxamide

Prepared using IM16 and IM29.

LC-MS (m/z) 340 (MH⁺); t_{R}=1.17 (Method A).

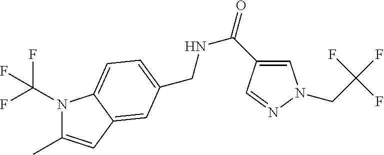

### 83: N-[[2-methyl-1-(trifluoromethyl)indol-5-yl]methyl]-1-(2,2,2-trifluromethyl)pyrazole-4-carboxamide

Prepared using IM18 and IM29.

LC-MS (m/z) 405 (MH⁺); t_{R}=1.72 (Method A).

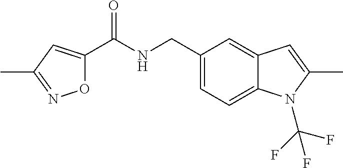

### 84: 3-methyl-N-[[2-methyl-1-(trifluoromethyl)indol-5-yl]methyl]isoxazole-5-carboxamide

Prepared using IM18 and 3-methyl-isoxazole-5-carboxylic acid.

LC-MS (m/z) 338 (MN⁺); t_{R}=1.70 (Method A).

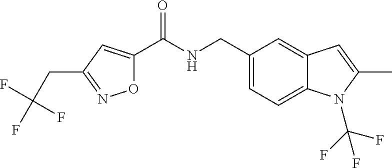

### 85: N-[[2-methyl-1-(trifluoromethyl)indol-5-yl]methyl]-3-(2,2,2-trifluoroethyl)isoxazole-5-carboxamide

Prepared using IM18 and IM35.

1H NMR (DMSO, 400 MHz) δ: 9.58-9.56 (1H, m), 7.52-7.49 (2H, m), 7.26-7.25 (1H, m), 7.14 (1H, s), 6.59 (1H, s), 4.53-4.51 (2H, d), 4.04-3.95 (2H, m), 2.50 (3H, m).

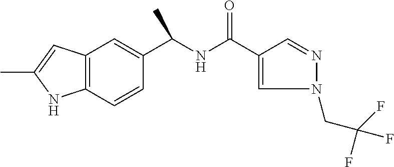

### 86: N-[(1R)-1-(2-methyl-1H-indol-5-yl)ethyl]-1-(2,2,2-trifluoroethyl)pyrazole-4-carboxamide

Prepared using IM9 and IM29.

LC-MS (m/z) 351 (MH⁺); t_{R}=1.28 (Method A).

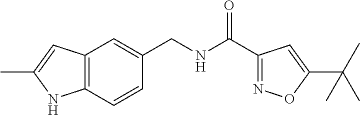

### 87: 5-tert-butyl-N-[(2-methyl-1H-indol-5-yl)methyl]isoxazole-3-carboxamide

Prepared using IM1 and 5-tert-butyl-isoxazole-3-carboxylic acid.

LC-MS (m/z) 312 (MH⁺); t_{R}=1.64 (Method A).

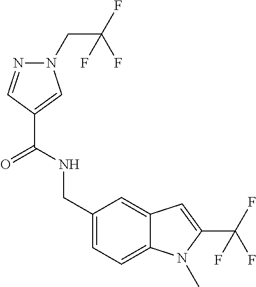

### 88: N-[[1-methyl-2-(trifluoromethyl)indol-5-yl]methyl]-1-(2,2,2-trifluoroethyl)pyrazole-4-carboxamide

Prepared using IM19 and IM29.

LC-MS (m/z) 405 (MH⁺); t_{R}=1.65 (Method A).

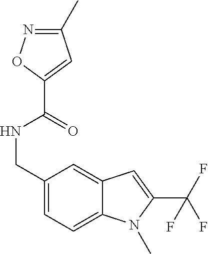

### 89: 3-methyl-N-[[1-methyl-2-(trifluoromethyl)indol-5-yl]methyl]isoxazole-5-carboxamide

Prepared using IM19 and 3-methyl-isoxazole-5-carboxylic acid.

LC-MS (m/z) 338 (MH⁺); t_{R}=1.62 (Method A).

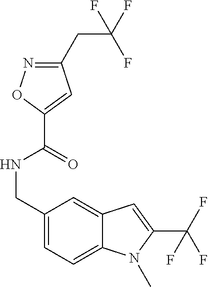

### 90: N-[[1-methyl-2-(trifluoromethyl)indol-5-yl]methyl]-3-(2,2,2-trifluoroethyl)isoxazole-5-carboxamide

Prepared using IM19 and IM35.

LC-MS (m/z) 406 (MH⁺); t_{R}=1.79 (Method A).

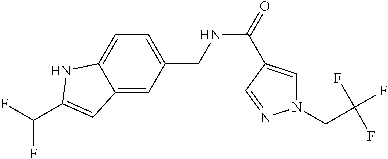

### 91: N-[[2-(difluoromethyl)-1H-indol-5-yl]methyl]-1-(2,2,2-trifluoroethyl)pyrazole-4-carboxamide

Prepared using IM20 and IM29.

LC-MS (m/z) 373 (MN⁺); t_{R}=1.21 (Method A).

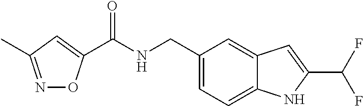

### 92: N-[[2-(difluoromethyl)-1H-indol-5-yl]methyl]-3-methyl-isoxazole-5-carboxamide

Prepared using IM20 and 3-methyl-isoxazole-5-carboxylic acid.

1H NMR (DMSO, 400 MHz) δ 11.72 (1H, br s), 9.42-9.39 (1H, m), 7.53 (1H, s), 7.39-7.37 (1H, m), 7.33-7.06 (2H, m), 6.93 (1H, s), 6.75-6.74 (1H, m), 4.50-4.49 (2H, m), 2.28 (3H, s).

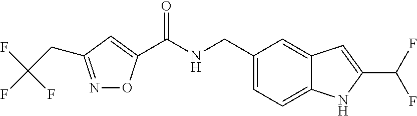

### 93: N-[[2-(difluoromethyl)-1H-indol-5-yl]methyl]-3-(2,2,2-trifluoroethyl)isoxazole-5-carboxamide

Prepared using IM20 and IM35.

1H NMR (DMSO, 400 MHz) δ 11.74 (1H, br s), 9.54-9.51 (1H, m), 7.55 (1H, s), 7.40-7.38 (1H, m), 7.33-7.06 (3H, m), 6.75 (1H, s), 4.52-4.51 (2H, m), 4.03-3.95 (2H, m).

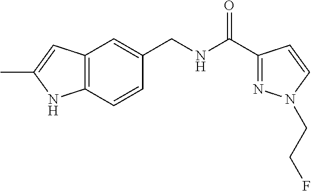

### 94: 1-(2-fluoroethyl)-N-[(2-methyl-1H-indol-5-yl)methyl]pyrazole-3-carboxamide

Prepared using IM1 and IM36.

LC-MS (m/z) 301 (MH⁺); t_{R}=1.09 (Method A).

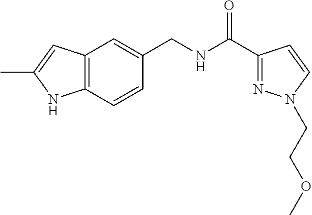

### 95: 1-(2-methoxyethyl)-N-[(2-methyl-1H-indol-5-yl)methyl]pyrazole-3-carboxamide

Prepared using INN and IM37.

LC-MS (m/z) 313 (MH⁺); t_{R}=1.12 (Method A).

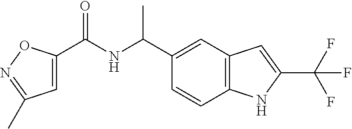

### 96: 3-methyl-N-[1-[2-(trifluoromethyl)-1H-indol-5-yl]ethyl]isoxazole-5-carboxamide

Prepared using IM21 and 3-methyl-isoxazole-5-carboxylic acid.

LC-MS (m/z) 339 (MH⁺); t_{R}=1.52 (Method A).

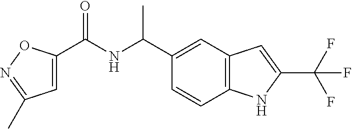

97: 3-methyl-N-[1-[2-(trifluoromethyl)-1H-indol-5-yl]ethyl]isoxazole-5-carboxamide

Prepared using IM22 and 3-methyl-isoxazole-5-carboxylic acid.

LC-MS (m/z) 339 (MH⁺); t_{R}=1.51 (Method A).

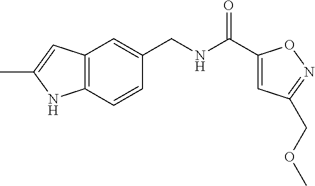

### 98: 3-(methoxymethyl)-N-[(2-methyl-1H-indol-5-yl)methyl]isoxazole-5-carboxamide

Prepared using IM1 and IM38.

LC-MS (m/z) 300 (MH⁺); t_{R}=1.13 (Method A).

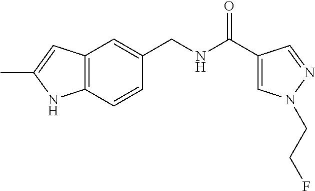

### 99: 1-(2-fluoroethyl)-N-[(2-methyl-1H-indol-5-yl)methyl]pyrazole-4-carboxamide

Prepared using IM1 and IM31.

LC-MS (m/z) 301 (MH⁺); t_{R}=0.98 (Method A).

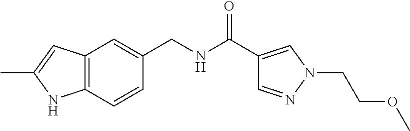

### 100: 1-(2-methoxyethyl)-N-[(2-methyl-1H-indol-5-yl)methyl]pyrazole-4-carboxamide

Prepared using IM1 and IM30.

LC-MS (m/z) 313 (MH⁺); t_{R}=1.02 (Method A).

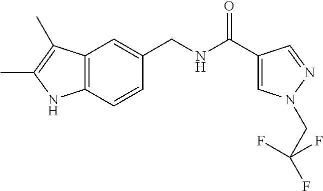

### 101: N-[(2,3-dimethyl-1H-indol-5-yl)methyl]-1-(2,2,2-trifluoroethyl)pyrazole-4-carboxamide

Prepared using IM8 and IM29.

LC-MS (m/z) 351 (MH⁺); t_{R}=1.33 (Method).

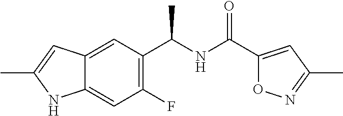

### 102: N-[(1R)-1-(6-fluoro-2-methyl-1H-indol-5-yl)ethyl]-3-methyl-isoxazole-5-carboxamide

Prepared using IM23 and 3-methyl-isoxazole-5-carboxylic acid. The title compound was purified by preparative HPLC (Method I).

1H NMR (DMSO, 400 MHz) δ 10.91 (1H, s), 9.23-9.21 (1H, m), 7.43-7.41 (1H, m), 7.01-6.95 (2H, m), 6.08 (1H, s), 5.42-5.35 (1H, m), 2.33 (3H, s), 2.29 (3H, s), 1.49-1.47 (3H, m).

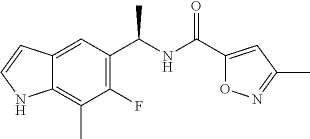

### 103: N-[(1R)-1-(6-fluoro-7-methyl-1H-indol-5-yl-ethyl]-3-methyl-isoxazole-5-carboxamide

Prepared using IM23 and 3-methyl-isoxazole-5-carboxylic acid. The title compound was purified by preparative HPLC (Method I).

1H NMR (DMSO, 400 MHz) δ 11.11 (1H, s), 9.23-9.21 (1H, m), 7.40-7.39 (1H, m), 7.31-7.30 (1H, m), 6.96 (1H, s), 6.40-6.39 (1H, m), 5.41-5.37 (1H, t), 2.38 (3H, s), 2.37 (3H, s), 1.50-1.48 (3H, d).

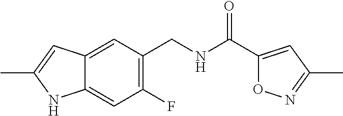

### 104: N-[(6-fluoro-2-methyl-1H-indol-5-yl)methyl]-3-methyl-isoxazole-5-carboxamide

Prepared using IN24 and 3-methyl-isoxazole-5-carboxylic acid.

1H NMR (DMSO, 400 MHz) δ 10.93 (1H, br s), 9.31-9.28 (1H, m), 7.32-7.31 (1H, m), 7.04-7.01 (1H, d), 6.94 (1H, s), 6.08 (1H, s), 4.50-4.49 (2H, m), 2.33 (3H, s), 2.28 (3H, s).

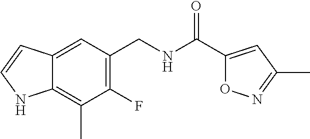

### 105: N-[(6-fluoro-7-methyl-1H-indol-5-yl)methyl]-3-methyl-isoxazole-5-carboxamide

Prepared using IM24 and 3-methyl-isoxazole-5-carboxylic acid. The title compound was purified by preparative HPLC (Method I).

1H NMR (DMSO, 400 MHz) δ 11.14 (1H, s), 9.29 (1H, br s), 7.31-7.29 (2H), 6.95 (1H, s), 6.40 (1H, s), 4.52-4.50 (2H, m), 2.38 (3H, s), 2.29 (3H, s).

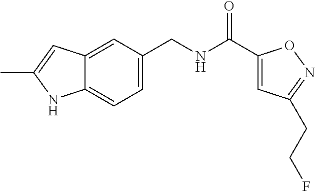

### 106: 3-(2-fluoroethyl)-N-[(2-methyl-1H-indol-5-yl)methyl]isoxazole-5-carboxamide

Prepared using IM1 and IM39.

LC-MS (m/z) 302 (MH⁺); t_{R}=0.58 (Method C).

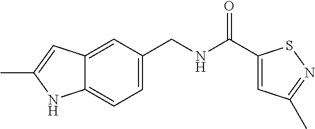

### 107: 3-methyl-N-[(2-methyl-1H-indol-5-yl)methyl]isothiazole-5-carboxamide

Prepared using IM1 and 3-methyl-isothiazole-5-carboxylic acid.

LC-MS (m/z) 286 (MH⁺); t_{R}=0.59 (Method C).

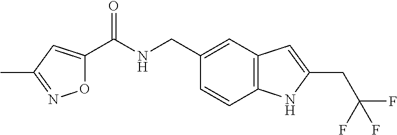

### 108: 3-methyl-N-[[2-(2,2,2-trifluoroethyl)-1H-indol-5-yl]methyl]isoxazole-5-carboxamide

Prepared using IM25 and 3-methyl-isoxazole-5-carboxylic acid.

LC-MS (m/z) 339 (MH⁺): t_{R}=0.64 (Method C).

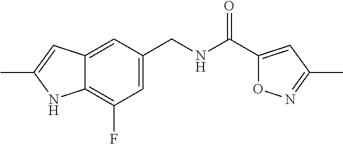

### 109: N-[(7-fluoro-2-methyl-1H-indol-5-yl)methyl]-3-methyl-isoxazole-5-carboxamide

Prepared using IM26 and 3-methyl-isoxazole-5-carboxylic acid. The title compound was purified by preparative HPLC (Method I).

1H NMR (DMSO, 400 MHz) δ 11.29 (1H, s), 9.39-9.36 (1H, m), 7.15 (1H, s), 6.93 (1H, s), 6.80-6.67 (1H, m), 6.16 (1H, s), 4.46-4.44 (2H, d), 2.36 (3H, s), 2.28 (3H, s).

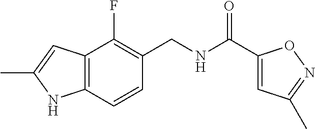

### 110: N-[(4-fluoro-2-methyl-1H-indol-5-yl)methyl]-3-methyl-isoxazole-5-carboxamide

Prepared using IM27 and 3-methyl-isoxazole-5-carboxylic acid.

1H-NMR (CDCl₃, 400 MHz) δ 7.99 (s, 1H), 7.24-7.02 (m, 2H), 6.81 (br s, 1H), 6.73 (s, 1H), 6.29-6.28 (m, 1H), 4.73-4.71 (q, 2H), 2.43 (3H, d), 2.33 (s, 3H).

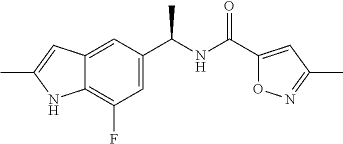

### 111: N-[(1R)-1-(7-fluoro-2-methyl-1H-indol-5-yl)ethyl]-3-methyl-isoxazole-5-carboxamide

Prepared using IM28 and 3-methyl-isoxazole-5-carboxylic acid.

1H NMR (DMSO, 400 MHz) δ 11.27 (1H, s), 9.21-9.19 (1H, d), 7.20 (1H, s), 6.93 (1H, s), 6.89-6.87 (1H, m), 6.16 (1H, s), 5.17-5.14 (1H, m), 2.36 (3H, s), 2.28 (3H, s), 1.49-1.48 (3H, d).

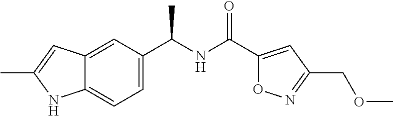

### 112: 3-(methoxymethyl)-N-[(1R)-1-(2-methyl-1H-indol-5-yl)ethyl]isoxazole-5-carboxamide

Compound IM38 (0.429 g, 2.73 mmol) was dissolved in N,N-Dimethylformamide (5.00 mL, 64.6 mmol) and tetrahydrofuran (40.0 mL). To this mixture was added N,N-Carbonyldiimidazole (0.488 g, 3.01 mmol) and the mixture was refluxed for 25 minutes. To this mixture was added IM9 (0.650 g, 3.73 mmol) and the mixture was refluxed for 1 hour. The mixture was evaporated to dryness. Flash chromatography (silica, EtOAc:heptanes 1:1) gave the title compound as a light yellow foam (0.22 g, 24%).

LC-MS (m/z) 315 (MH⁺); t_{R}=0.60 (Method C).

The following compounds were prepared analogously:

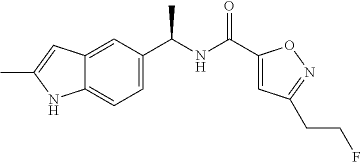

### 113: 3-(2-fluoroethyl)-N-[(1R)-1-(2-methyl-1H-indol-5-yl)ethyl]isoxazole-5-carboxamide

Prepared using IM9 and IM39.

1H NMR (500 MHz, DMSO) δ 10.83 (s, 1H), 9.23 (d, 1H), 7.39 (s, 1H), 7.21 (d, 1H), 7.09 (s, 1H), 7.04 (m, 1H), 6.08 (s, 1H), 5.25-5.13 (m, 1H), 4.80-4.78 (t, 1H), 4.71-4.68 (t, 1H), 3.14-3.07 (m, 2H), 2.37 (s, 3H), 1.52-1.50 (d, 3H).

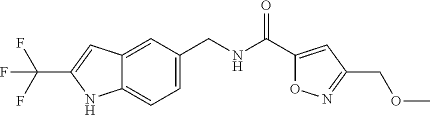

### 114: 3-(methoxymethyl)-N-[[2-(trifluoromethyl)-1H-indol-5-yl]methyl]isoxazole-5-carboxamide

Prepared using IM5 and IM38.

1H NMR (600 MHz, DMSO) δ 12.24 (s, 1H), 9.53 (m, 1H), 7.61 (s, 1H), 7.45 (d, 1H), 7.28 (m, 1H), 7.09 (s, 1H), 7.01 (s, 1H), 4.53 (s, 4H), 3.35 (s, 3H).

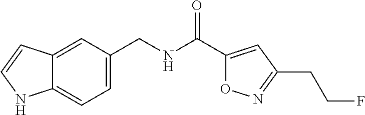

### 115: 3-(2-fluoroethyl)-N-(1H-indol-5-ylmethyl)isoxazole-5-carboxamide

Prepared using IM12 and IM39.

LC-MS (m/z) 288 (MH⁺); t_{R}=0.51 (Method C).

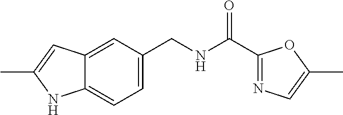

### 116: 5-methyl-N-[(2-methyl-1H-indol-5-yl)methyl]oxazole-2-carboxamide

To a solution of 5-methyl-oxazole-2-carboxylic acid ethyl ester (300 mg, 1.94 mmol) in toluene (5 mL) at 0° C. was added Al(CH₃)₃ (1.5 mL, 2.90 mmol). The mixture was stirred for 5 min. Compound IM1 (72 mg, 2.32 mmol) was added and then the mixture was heated to 110° C. for 1 h in a sealed tube. The reaction was quenched with 5% aqueous HCl (4 mL) and then diluted with EtOAc (35 mL). The organic layer was washed with water (2×15 mL). The organic layer was evaporated to dryness. Flash chromatography (silica, EtOAc:heptanes 1:1) gave the title compound as a yellow solid (165 mg, 33%).

LC-MS (m/z) 270 (MH⁺); t_{R}=0.55 (Method C).

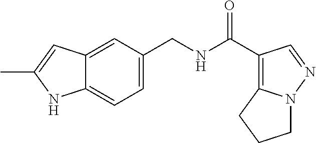

### 117: N-[(2-methyl-1H-indol-5-yl)methyl]-5,6-dihydro-4H-pyrrolo[1,2-b]pyrazole-3-carboxamide

Prepared using IM1 and IM40.

1H NMR (DMSO-d6, 400 MHz) δ 10.79 (1H, br s), 8.31-8.28 (1H, m), 7.91 (1H, s), 7.28 (1H, s). 7.19-7.17 (1H, d), 6.94-6.92 (1H, d), 6.05 (1H, s), 4.43-4.41 (2H, m), 4.06-4.03 (2H, m), 2.99-2.95 (2H, m), 2.58-2.50 (2H, m), 2.35 (3H, s).

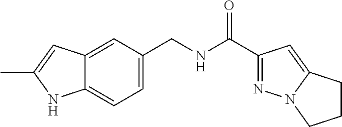

### 118: N-[(2-methyl-1H-indol-5-yl)methyl]-5,5-dihydro-4H-pyrrolo[1,2-b]pyrazole-2-carboxamide

Prepared using IM1 and IM41.

1H NMR (DMSO, 400 MHz) δ 10.79 (1H, s), 8.38-8.35 (1H, m), 7.28 (1H, s), 7.17-7.15 (1H, d), 6.95-6.93 (1H, d), 6.36 (1H, s), 6.04 (1H, s), 4.43-4.41 (2H, m), 4.10-4.07 (2H, m), 2.86-2.82 (2H, m), 2.57-2.50 (2H, m), 2.34 (3H, s).

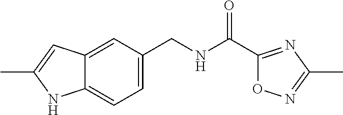

### 119: 3-methyl-N-[(2-methyl-1H-indol-5-yl)methyl]-1,2,4-oxadiazole-5-carboxamide

A mixture of compound IM1 (160 mg, 1 mmol) and 3-methyl-[1,2,4]oxadiazole-5-carboxylic acid ethyl ester (156 mg, 1 mmol) in anhydrous EtOH (3 mL) was stirred at reflux for 15 h. The mixture was evaporated to dryness and the residue was purified by preparative TLC (silica, EtOAc:petroleum ether 1:1) to give the title compound as a yellow solid (210 mg, 78%). 1H NMR (CDCl₃ 400 MHz) δ 7.87 (s, 1H), 7.40 (s, 1H), 7.22-7.16 (m, 2H), 7.02-6.98 (m, 1 H), 6.13 (s, 1H), 4.62 (m, 2H), 2.38 (s, 3H), 2.36 (s, 3H).

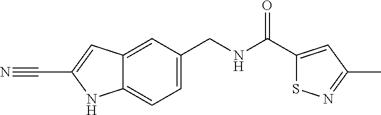

### 120: N-[(2-cyano-1H-indol-5-yl)-methyl]-3-methyl-isothiazole-5-carboxamide

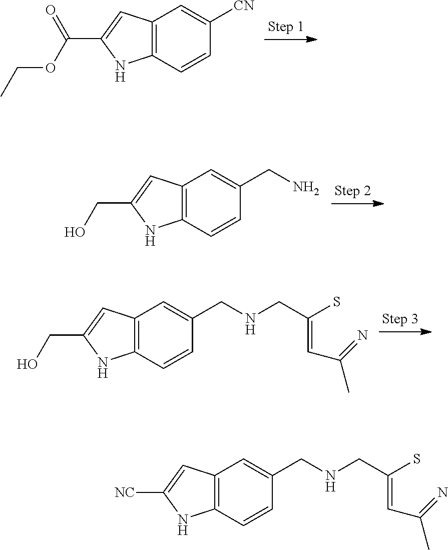

### Step 1:

Lithium tetrahydroaluminate (6.00 g, 158 mmol) was suspended in THF (400 mL) and heated to reflux. A mixture of 5-cyano-1H-indole-2-carboxylic acid ethyl ester (7.90 g, 36.9 mmol) in THF (125 mL) was added dropwise over 30 min. The mixture was refluxed for another 20 min. The mixture was cooled to 0° C. and water (12 mL), NaOH solution (4M, 6 mL) and the water (30 mL) was sequentially carefully added. MgSO₄ was added and the mixture stirred for 5 min. The mixture was filtered and the filtrate was evaporated to dryness. Flash chromatography (silica, EtOAc:EtOH:tri ethyl amine 70:25:5) gave crude (5-aminomethyl-1H-indol-2-yl)methanol as a yellow oil (1.25 g, 16%) which was used without further purification.

### Step 2:

3-methyl-isothiazole-5-carboxylic acid (0.50 g, 3.49 mmol), N,N,N′,N′-tetramethyl-O-(7-azabenzotriazol-1-yl)uronium Hexafluorophosphate (1.38 g, 3.63 mmol), triethyl amine (1.25 mL, 8.97 mmol) were stirred in DMF (5 mL) for 15 min at room temperature. This mixture was added dropwise over 5 min at room temperature to a solution of the 1.25 g crude amine from step 1 in THF (25 mL). The resulting mixture was poured into brine and subsequently extracted with EtOAc. The combined organic layers were washed with brine, dried over MgSO₄ and evaporated to dryness. Flash chromatography (silica, EtOAc) gave 3-Methyl-isothiazole-5-carboxylic acid (2-hydroxymethyl-1H-indol-5-ylmethyl)-amide as a beige foam (0.807 g, 7% from 5-cyano-1H-indole-2-carboxylic acid ethyl ester).

### Step 3:

3-Methyl-isothiazole-5-carboxylic acid (2-hydroxymethyl-1H-indol-5-ylmethyl)-amide (0.800 g, 2.65 mmol) was dissolved in a mixture of dimethyl sulfoxide (25.0) and THF (25.0 mL). 1-Hydroxy-1,2-benziodoxol-3(1H)-one 1-Oxide (0.815 g, 2.91 mmol) was added and the mixture was stirred at room temperature overnight. The mixture was poured into brine and extracted with EtOAc. The organic layer was washed with brine, dried over MgSO₄ and evaporated to dryness to give 1.33 g light-brown powder. This powder was dissolved in THF (20.0 mL).

In another flask 1-hydroxy-1,2-benziodoxol-3(1H)-one 1-Oxide (1.11 g, 3.98 mmol) was dissolved in ammonia in Water (13M, 20 mL). When all the solids had dissolved acetonitrile (15 mL) was added and the mixture was stirred for 5 min. To this mixture was added the mixture of the light-brown powder in THF dropwise and the resulting mixture was stirred for 3 days at room temperature. The mixture was poured into brine. The resulting mixture was extracted with EtOAc and the combined organic layers were washed with brine, dried over MgSO₄ and evaporated to dryness. Flash chromatography (silica, EtOAc:heptanes 4:1) gave the title compound 120 as colorless crystals (0.51 g, 61% from 3-Methyl-isothiazole-5-carboxylic acid (2-hydroxymethyl-1H-indol-5-ylmethyl)-amide). LC-MS (m/z) 297 (MH⁺); t_{R}=0.52 (Method C).

1H NMR (DMSO 600 MHz) δ 12.36 (br s, 1H), 9.37-9.34 (m, 7.72 (s, 1H), 7.61 (s, 1H), 7.46-7.44 (m, 1H), 7.36-7.33 (m, 2H), 4.55-4.54 (m, 2H), 2.46 (s, 3H).

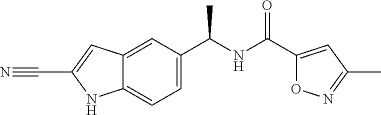

### 121: N-[(1R)-1-(2-cyano-1H-indol-5-yl)ethyl]-3-methyl-isoxazole-5-carboxamide

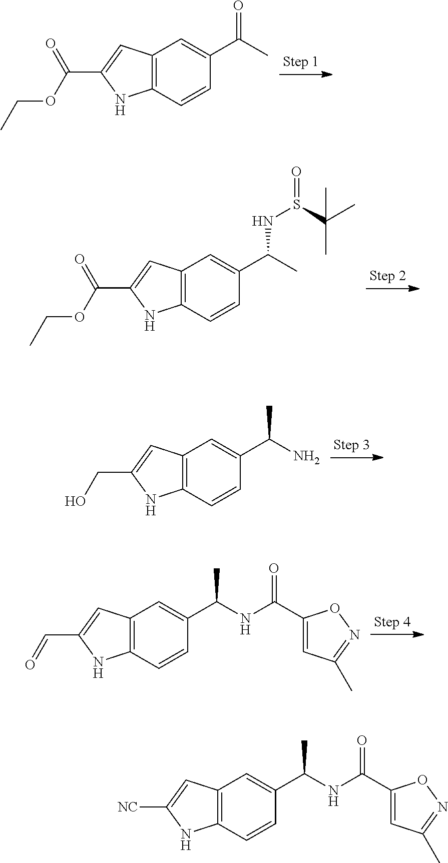

### Step 1:

5-Acetyl-1H-indole-2-carboxylic acid ethyl ester (15.4 g, 59.9 mmol) was dissolved in THF (500). S(−)-2-Methyl-2-propanesulfinamide (10.0 g, 82.5 mmol) and Ti(OEt)₄ (40.0 mL, 193 mmol) was added and the mixture was heated to reflux for 2 days. The mixture was diluted with 750 ml EtOAc and poured onto brine. The mixture was filtered through a plug of celite. The organic layer was washed with brine, dried over MgSO₄ and evaporated to dryness. Flash chromatography (silica, EtOAc:heptanes 1:1) gave the crude imine as a yellow powder (17.29). This material was dissolved in THF (300 mL) and added dropwise to L-Selectride (R) in THF (1.00 M, 180.0 mL) over 15 minutes at keeping the internal temperature at −48 to −46° C. The mixture was stirred at −50° C. for 20 minutes and was then allowed to reach 0° C. The cold mixture was poured on saturated NH₁Cl solution and extracted with EtOAc. The combined organic layers were washed with brine, dried over MgSO₄ evaporated to dryness. Flash chromatography (silica, EtOAc) gave 5-[(R)-1-(S)-2-methyl-propane-2-sulfinylamino)-ethyl]-1H-indole-2-carboxylic acid ethyl ester as a light-brown foam (10.5 g, 50%).

### Step 2:

5-[(R)-1-((S)-2-Methyl-propane-2-sulfinylamino)-ethyl]-1H-indole-2-carboxylic acid ethyl ester (10.5 g, 29.6 mmol) was dissolved in MeOH (250 mL). A mixture of 12 M of Hydrogen chloride in Water (10 mL) and Water (10 mL) was added dropwise over 2 min. The mixture was stirred for 4 hours at room temperature. To this mixture was added K₂CO₃ (10.24 g, 74.12 mmol) and the resulting mixture was stirred at room temperature overnight. The volume was reduced in vacuo to approx 50 mL and subsequently diluted with EtOAc (500 mL). This mixture was dried over MgSO₄. filtered and evaporated to dryness. The residue (16 g brown oil) was dissolved in THF (200 mL) and carefully added dropwise to a suspension of lithium tetrahydroaluminate (2.50 g, 65.9 mmol) in THF (200 mL) over 20 min at 6-16° C. (the reaction flask was cooled in ice/water). Then the mixture was stirred at 10° C. for 30 minutes. To this mixture was carefully added 5 ml water followed by 2.5 ml sodium hydroxide-solution (4M) and 10 ml water. The mixture was stirred for 5 minutes and then a generous amount of MgSO₄ was added. The mixture was filtered and evaporated to dryness. Flash chromatography (silica, EtOAc:EtOH:triethyl amine 70:25:5) gave [5-((R)-1-amino-ethyl)-1H-indol-2-yl]-methanol as a light-brown oil (4.72 g, 71%).

### Step 3:

3-Methyl-isoxazole-5-carboxylic acid (0.900 g, 7.08 mmol) was dissolved in N,N-Dimethylformamide (10.0 mL). N,N,N′,N′-Tetramethyl-O-(7-azabenzotriazol-1-yl)uronium Hexafluorophosphate (2.80 g, 7.36 mmol) and triethylamine (2.50 mL, 17.9 mmol) was added. The mixture was stirred at room temperature for 20 minutes. The resulting mixture was added dropwise to a mixture of [5-((R)-1-Amino-ethyl)-1H-indol-2-yl]-methanol (1.66 g, 7.44 mmol) and triethyl amine (2.0 mL) in N,N-dimethylformamide (10 mL) and then left stirring at room temperature overnight. The mixture was poured into brine and extracted with EtOAc. The combined organic layers were washed with brine and dried over MgSO₄. Flash chromatography (silica, EtOAc) gave the crude amide as a red oil (1.8 g). This residue was dissolved in a mixture of dimethylsulfoxide (50 mL) and THF (50). 1-Hydroxy-1,2-benziodoxol-3(1H)-one 1-Oxide (2.18 g, 7.79 mmol) was added and the mixture was stirred at room temperature overnight. The mixture was poured into brine and extracted with EtOAc. The combined organic layers were washed with brine, dried over MgSO₄, filtered and evaporated to dryness. Flash chromatography (silica, EtOAc) gave 3-methyl-isoxazole-5-carboxylic acid [(R)-1-(2-formyl-1H-indol-5-yl)-ethyl]-amide as a colorless powder (0.365 g, 17%).

### Step 4:

1-Hydroxy-1,2-benziodoxol-3(1H)-one 1-Oxide (0.508 g, 1.82 mmol) was dissolved in 13.0 M of Ammonia in Water (10 mL). Acetonitrile (8 mL) was added. To this mixture was added a solution of 3-methyl-isoxazole-5-carboxylic acid [(R)-1-(2-formyl-1H-indol-5-yl)-ethyl]-amide (0.360 g, 1.21 mmol) in THF (15 mL) dropwise over 3 min. The mixture was poured into brine and extracted with EtOAc. The combined organic layers were washed with brine, dried over MgSO₄ and evaporated to dryness. Flash chromatography (silica, EtOAc:heptanes 4:1) gave the title compound 121 as colorless crystals (0.276 g, 77%). LC-MS (m/z) 295 (MH⁺); t_{R}=0.88 (Method A).

1H NMR (500 MHz, DMSO) δ 12.33 (br s, 1H), 9.31-9.29 (m, 1H), 7.65 (s, 1H), 7.43-7.36 (m, 3H), 6.95 (s, 1H), 5.23-5.20 (m, 1H), 2.29 (s, 3H), 1.53-1.52 (d, 3H).

### In Vitro Assays

The nicotinic acetylcholine receptor α7 is a calcium-permeable ion channel, whose activity can be measured by over expression in mammalian cells or oocytes. These two individual assays are described in Example 2 and 3 respectively.

### Example 2

### α7 NNR flux Assay

In this version of the assay, the human α7 receptor is stably expressed in the rat GH4C1 cell line. The assay was used to identify positive allosteric modulators (PAMs) of the α7 receptor. Activation of the channel was measured by loading cells with the calcium-sensitive fluorescent dye Calcium-4 (Assay kit from Molecular Devices), and then measuring real-time changes in fluorescence upon treatment with test compounds.

The cell line ChanClone GH4C1-nAChRalpha7 from Genionics was seeded from frozen stock in 384-well plates in culture media 2-3 days before experiment to form an approximately 80% confluent layer on the day of experiment.

### Cell Plating and Dye Loading

The cell culture were split into “22.5 cm×22.5 cm”-plates with approximately 100×10³ cells/cm². After four days incubation in a humidified incubator at 37° C. and 5% CO₂, it had grown to an 80-90% confluent layer, and the cells were harvested.

Culture Media:

500 mL DMEM/F12 (Gibco 31331)

50 mL FBS (Gibco 10091-155, lot 453269FD)

5 mL Sodium Pyruvate (Gibco 11360)

5 mL Pen/Strep (Gibco 15140)

0.1 mg/mL G-418 (Gibco 11811-064)

Two or three days before the experiment the cells were seeded in 384 well plates from Greiner bio-one (781946, CELLCOAT, Poly-D-Lysine, black, μClear).

The media was poured off and the plate washed with PBS and left to drain. 5 mL Trypsin was added, cells were washed and incubated (at room temperature) for about 10 seconds. Trypsin was poured of quickly and the cells were incubated for 2 minutes at 37° C. (if the cells were not already detached). Cells were resuspended in 10 mL culture media and transfered to 50 mL tubes.

The cell suspension was counted (NucleoCounter, total cell count) from the first plates to estimate the total cell number of the whole batch.

The cells were seeded in 384 well plates with 30 μL/well (30000 cells/well) while stirring the cell suspension or otherwise preventing the cells from precipitating.
The plates were incubated at room temperature for 30-45 minutes.
The plates were placed in incubator for two days (37° C. and 5% CO2).

### Loading the Cells

The loading buffer was 5% v/v Calcium-4 Kit and 2.5 mM Probenecid in assay buffer.

190 mL assay buffer

10 mL Kit-solution

2 mL 250 mM Probenecid

This volume was enough for 3×8 cell plates.

Culture media were removed from the cell plates and 20 μL loading buffer was added in each well. The cell plates were placed in trays and incubated 90 minutes in the incubator (37° C.). Thereafter the plates were incubated 30 minutes at room temperature, still protected from light. Now the cell plates were ready to run in the Functional Drug Screening System (FDSS) The assay buffer was HBSS with 20 mM HEPES, pH 7.4 and 3 mM CaCl₂.

### FDSS Ca Assay

200 nL 10 mM compound solution in DMSO was diluted in 50 μL assay buffer. The final test concentrations in the cell plates were 20-10-5-2.5-1.25-0.625-0.312-0.156-0.078-0.039 μM. Assay buffer and 3 μM PNU-120596 were used for control:

The agonist acetylcholine was added to a final concentration of 20 μM (˜EC100).
In the FDSS7000 the Ex480-Em540 was measured with 1 second intervals. The baseline was made of 5 frames before addition of test compounds, and 95 frames more were made before addition of acetylcholine. The measurement stopped 30 frames after the 2^{nd} addition.
Raw data for each well were collected as “the maximum fluorescence count” in the interval 100-131 seconds and as “the average fluorescence count” in the interval 96-100 seconds.
The positive allosteric modulation in the 2^{nd} addition was the enhancement of agonist response with test compound compared to agonist alone.

Results were calculated as % modulation of test compound compared to the reference PNU-120596 set to 100%. From these data EC₅₀ curves were generated giving EC₅₀, hill and maximum stimulation.

The compounds of the present invention characterized in the flux assay generally possess EC₅₀ values below 20.000 nM or less such as below 10.000 nM. Many compounds, in fact have EC₅₀ values below 5.000 nM. Table 1 shows EC₅₀ values for exemplified compounds of the invention.

TABLE 1

| Compound | EC₅₀ (nM) |
|---|---|
| 1 | 2700 |
| 2 | 9800 |
| 3 | 130 |
| 4 | 2900 |
| 5 | 4400 |
| 6 | 520 |
| 7 | 1400 |
| 8 | 3200 |
| 9 | 1800 |
| 10 | 4000 |
| 11 | 2400 |
| 12 | 9100 |
| 13 | 530 |
| 14 | 7400 |
| 15 | 9600 |
| 16 | 11000 |
| 17 | 11000 |
| 18 | 6700 |
| 19 | 4600 |
| 20 | 3300 |
| 21 | 9500 |
| 22 | 7200 |
| 23 | 7100 |
| 24 | 340 |
| 25 | 4700 |
| 26 | 7367 |
| 27 | 440 |
| 28 | 2967 |
| 29 | 9250 |
| 30 | 5800 |
| 31 | nd |
| 32 | 11000 |
| 33 | 2633 |
| 34 | 900 |
| 35 | 2450 |
| 36 | 1495 |
| 37 | 9800 |
| 38 | 5900 |
| 39 | 9100 |
| 40 | 3500 |
| 41 | 7700 |
| 42 | 5600 |
| 43 | 2200 |
| 44 | 6100 |
| 45 | 1500 |
| 46 | 3500 |
| 47 | >20000 |
| 48 | 1700 |
| 49 | 6500 |
| 50 | 8500 |
| 51 | 6100 |
| 52 | 5300 |
| 53 | 6000 |
| 54 | >20000 |
| 55 | 7300 |
| 56 | 4300 |
| 57 | 3867 |
| 58 | 8567 |
| 59 | 7700 |
| 60 | 4100 |
| 61 | 2325 |
| 62 | 9100 |
| 63 | 5800 |
| 64 | 9300 |
| 65 | 5000 |
| 66 | 8800 |
| 67 | 6100 |
| 68 | 2100 |
| 69 | 4200 |
| 70 | 9400 |
| 71 | 2900 |
| 72 | 7600 |
| 73 | 2800 |
| 74 | 6000 |
| 75 | 2100 |
| 76 | 8200 |
| 77 | 10500 |
| 78 | 610 |
| 79 | 560 |
| 80 | 5700 |
| 81 | 6000 |
| 82 | 980 |
| 83 | 11000 |
| 84 | 6000 |
| 85 | 7700 |
| 86 | >20000 |
| 87 | 2600 |
| 88 | 6200 |
| 89 | 5600 |
| 90 | 5000 |
| 91 | 1700 |
| 92 | 8300 |
| 93 | 5400 |
| 94 | 9500 |
| 95 | 7700 |
| 96 | 4200 |
| 97 | 5200 |
| 98 | 3500 |
| 99 | 6400 |
| 100 | 8500 |
| 101 | 2900 |
| 102 | 1000 |
| 103 | 330 |
| 104 | 4200 |
| 105 | 2900 |
| 106 | 1700 |
| 107 | 560 |
| 108 | 7700 |
| 109 | 3100 |
| 110 | 6500 |
| 111 | 1300 |
| 112 | 1100 |
| 113 | 790 |
| 114 | 5000 |
| 115 | 4300 |
| 116 | 9900 |
| 117 | 7000 |
| 118 | 7200 |
| 119 | 7100 |
| 120 | 3300 |
| 121 | 4800 |

Selected compounds of the invention were tested in the more comprehensive oocyte assay (Example 3). Compounds No. 34,47 and 86 were tested subsequently in the oocyte assay with the result of a significantly better NNR PAM activity in this assay.

### Example 3

### α7 NNR oocyte Assay

Expression of α7 nACh Receptors in Xenopus oocytes.

Oocytes were surgically removed from mature female Xenepus laevis anaesthetized in 0.4% MS-222 for 10-15 min. The oocytes were then digested at room temperature for 2-3 hours with 0.5 mg/ml collagenase (type IA Sigma-Aldrich) in OR2 buffer (82.5 mM NaCl, 2.0 mM KCl, 1.0 mM MgCl₂ and 5.0 mM HEPES, pH 7.6). Oocytes avoid of the follicle layer were selected and incubated for 24 hours in Modified Barth's Saline buffer (88 mM NaCl, 1 mM KO, 15 mM HEPES, 2.4 mM NaHCO₃, 0.41 mM CaCl₂, 0.82 mM MgSO₄, 0.3 mM Ca(NO₃)₂) supplemented with 2 mM sodium pyruvate, 0.1 μg/l penicillin and 0.1 μg/l streptomycin. Stage 1V oocytes were identified and injected with 4.2-48 nl of nuclease free water containing 0.1-1.2 ng of cRNA coding for human α7 nACh receptors or 3.0-32 ng of cRNA coding for rat α7 nACh receptors and incubated at 18° C. for 1-10 days when they were used for electrophysiological recordings.

Electrophysiological Recordings of α7 nACh Receptors Expressed in Oocytes.

Oocytes were used for electrophysiological recordings 1-10 days after injection. Oocytes were placed in a 1 ml bath and perfused with Ringer buffer (115 mM NaCl, 2.5 mM KCl, 10 mM HEPES, 1.8 mM CaCl₂, 0.1 mM MgCl₂, pH 7.5). Cells were impaled with agar plugged 0.2-1 MΩ electrodes containing 3 M KCl and voltage clamped at −90 mV by a GeneClamp 500B amplifier. The experiments were performed at room temperature. Oocytes were continuously perfused with Ringer buffer and the drugs were applied in the perfusate. ACh (30 μM) applied for 30 sec were used as the standard agonist for activation of the α7 nACh receptors. In the standard screening set-up the new test compound (10 μM or 30 μM) were applied for 1 min of pre-application allowing for evaluation of agonistic activity followed by 30 sec of co-application with ACh (30 μM) allowing for evaluation of PAM activity. The response of co-application was compared to the agonistic response obtained with ACh alone. The drug induced effects on both the peak response and the total charge (AUC) response were calculated thus giving the effect of drug induced PAM activity as fold modulation of the control response.

For more elaborate studies doses-response curves were performed for evaluation of max-fold modulation and EC₅₀ values for both peak and AUC responses. Selected compounds of the present invention (named “a”, “b”, “c” and “d”) have been tested giving dose-response curves as indicated in Table 2.

TABLE 2

| | | | EC₅₀ AUC | Max Fold |
|---|---|---|---|---|
| Compound | EC₅₀ peak (nM) | Max Fold Peak | (nM) | AUC |
| “a” | 8700 | 11 | 7000 | 6.4 |
| “b” | 6000 | 85 | 12000 | 160 |
| “c” | 5000 | 45 | 11000 | 100 |
| “d” | 700 | 42 | 5000 | 86 |

### Comparative Example Illustrating KCNQ2 Opening Effect

The inventors have found that compounds of the invention possess beneficial properties relative to known compounds which are PAMs of the NNRs. In particular, compounds of the invention have shown to be beneficial in terms of reduced effect as KCNQ channel openers compared to prior art compounds. This has been illustrated by experimental data summarized in Table 3.

### Example 4

### 86Rb Flux in KCNQ2 Expressing Cells

Chinese hamster ovary cells (CHO-K1) were stably transfected with human KCNQ2 inserted into the pCl-neo vector (Promega). Cells were plated at 2×105 cells/ml in 96-well plates in 100 μl media (MEM Alpha media, 10% FBS, 1% P/S, 1% L-Glu, and 1 mg/ml G418). At the time of plating the cells were furthermore loaded with 1 μCi/ml 86Rb over-night. On the subsequent day, the cells were washed with 3×50 μl assay buffer (HBSS+10 mM HEPES, pH 7.4) and incubated in 50 μl buffer with retigabine for 30 minutes at 37° C. Following this preincubation, the cell membrane was depolarized for 30 minutes at 37° C. with 15 mM KCl (final concentration) in assay buffer supplemented with the appropriate amount of compound to maintain drug concentration. The supernatant was removed and placed in pony-vials with 0.5 ml scintillation liquid and counted. The dose-dependent effect of the investigated compounds on the efflux of 86Rb through the hKCNQ2 channels were calculated according to the four parameter logistic equation: Y=Bottom+(Top-Bottom)/(1+10̂((LogEC50-X)*HillSlope)), where X is the logarithm of concentration, Y is the response, and HillSlope is the hill coefficient.

A representative selection of compounds of the invention were tested in the assay in addition to 6 comparative examples wherein 0 has been substituted with a 6-membered ring comprising up to two N atoms. Comparative 1-4 has been disclosed in WO 2009/127678 or WO 2009/127679 and comparative 5-6 are close analogues. Table 3 shows EC₅₀ values and Max effect determined in % of the effect of 30 mM KCl. These data clearly indicates that compounds of the invention benefit from reduced effect as openers of KCNQ channels.

Comparative compounds (denoted C1-C6) are illustrated below:

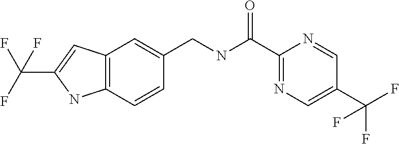

### C1: 5-Trifluoromethyl-pyrimidine-2-carboxylic acid (2-trifluoromethyl-1H-indol-5-ylmethyl)amide

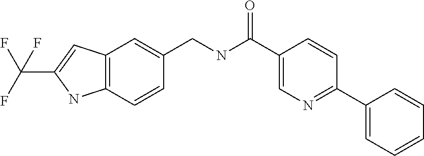

### C2: 6-Phenyl-N-(2-trifluoromethyl-1H-indol-5-ylmethyl)-nicotinamide

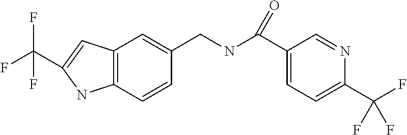

### C3: 6-Trifluoromethyl-N-(2-trifluoromethyl-1H-indol-5-ylmethyl)-nicotinamide

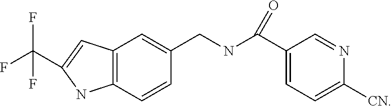

### C4: 6-Cyano-N-(2-trifluoromethyl-1H-indol-5-ylmethyl)-nicotinamide

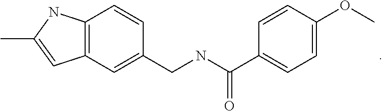

### C5: 4-Methoxy-N-(2-methyl-1H-indol-5-ylmethyl)-benzamide

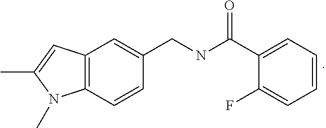

### C6: N-(1,2-Dimethyl-1H-indol-5-ylmethyl)-2-fluoro-benzamide

TABLE 3

| Compound | EC₅₀ (nM) | Max effect (%) |
|---|---|---|
| C1 | >50000 | 34 |
| C2 | >50000 | 31 |
| C3 | 17000 | 46 |
| C4 | >50000 | 15 |
| C5 | >50000 | 17 |
| C6 | >50000 | 13 |
| 1 | >50000 | 2.0 |
| 24 | >50000 | 5.0 |
| 28 | 100000 | 5.7 |
| 29 | >50000 | 4.0 |
| 30 | >50000 | 9.6 |
| 31 | >50000 | 4.8 |
| 34 | 100000 | 1.0 |
| 35 | 100000 | 7.5 |
| 36 | 100000 | 3.3 |
| 40 | >50000 | 2.4 |
| 61 | 100000 | −5 |
| 65 | 100000 | 8.1 |
| 66 | >50000 | 3.7 |
| 67 | >50000 | 5.3 |
| 96 | >50000 | 5.4 |

1. A compound according to formula I 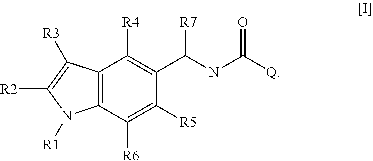
wherein R1 represents H, trifluoromethyl, difluoromethyl, C₁₋₄alkyl, C₂₋₄alkenyl or C₂₋₄alkynyl;
R2 represents H, C₁₋₄alkyl, C₂₋₄alkenyl, C₂₋₄alkynyl, halogen or cyano, wherein said C₁₋₄alkyl, C₂₋₄alkenyl or C₂₋₄alkynyl is optionally substituted with one or more substituents selected from chlorine and fluorine;
R3, R4, R5 and R6 are selected independently from H, C₁₋₄alkyl, C₂₋₄alkenyl, C₂₋₄alkynyl, halogen and cyano, wherein said C₁₋₄alkyl, C₂₋₄alkenyl or C₂₋₄alkynyl is optionally substituted with one or more substituents selected from chlorine and fluorine;
R7 represents H, methyl, trifluoromethyl or hydroxymethyl;
Q represents a heteroaryl with 5 ring atoms, wherein 1, 2 or 3 ring atoms are selected independently from O, N and S, wherein said heteroaryl may be optionally substituted on its carbon atoms with one or more substituents represented by R10, and provided that said heteroaryl cannot be 1,2,3 triazolyl or imidazolyl;
each R10 is independently selected from C₁₋₄alkyl, C₂₋₄alkenyl, C₂₋₄alkynyl, C₁₋₄-fluoroalkoxy, halogen and oxo, wherein said C₁₋₄alkyl, C₂₋₄alkenyl or C₂₋₄alkynyl is optionally substituted with one or more substituents selected from fluorine, C₁₋₄alkoxy and C₁₋₄fluoroalkoxy;
if one or more ring atoms in said heteroraryl are N atoms these may, when valency allows, individually be optionally substituted with a substituent represented by R11, wherein each R11 is independently selected from C₁₋₄alkyl and a monocyclic saturated ring moiety having 4-6 ring atoms wherein one of said ring atoms may be O and the rest is C, and wherein said C₁₋₄ alkyl may be optionally substituted with one or more substituents selected from fluorine, C₁₋₄alkoxy and C₁₋₄fluoroalkoxy;
when Q is a pyrazolyl at least one of the N atoms in said pyrazolyl must be substituted with R11;
two R10 or one R10 and one R11 may, when sitting on neighbouring ring atoms and when represented by C₁₋₄alkyl or be linked together by a carbon bond to form a fused ring system;
or a pharmaceutically acceptable salt thereof;
with the proviso that the compound of formula [I] is other than
furan-2-carboxylic acid (2-methyl-1H-indol-5-ylmethyl)-amide;
furan-2-carboxylic acid (1,2-dimethyl-1H-indol-5-ylmethyl)-amide;
thiophene-2-carboxylic acid (1,2-dimethyl-1H-indol-5-ylmethyl)-amide;
thiophene-2-carboxylic acid (2-methyl-1H-indol-5-ylmethyl)-amide.

2. The compound according to claim 1, wherein R1 represents H, trifluoromethyl, difluoromethyl or C₁₋₂alkyl;
R2 represents H, C₁₋₂alkyl or cyano, wherein said C₁₋₂alkyl is optionally substituted with one or more fluorine;
R3, R4, R5 and R6 are selected independently from H, methyl and fluorine;
R7 represents H, methyl or trifluoromethyl;
Q represents a heteroaryl with 5 ring atoms, wherein 1, 2 or 3 ring atoms are selected independently from O, N and S, wherein said heteroaryl may be optionally substituted on its carbon atoms with one or more substituents represented by R10 and provided that said heteroaryl cannot be 1,2,3 triazolyl or imidazolyl;
each R10 is independently selected from C₁₋₄alkyl, C₁₋₂alkoxy, halogen and oxo, wherein said C₁₋₄alkyl is optionally substituted with one or more substituents selected from fluorine and C₁₋₂ alkoxy;
if one or more ring atoms in said heteroraryl are N atoms these may, when valency allows, individually be optionally substituted with a substituent represented by R11, wherein each R11 is independently selected from C₁₋₄alkyl and a monocyclic saturated ring moiety having 4-6 ring atoms, wherein one of said ring atoms may be O and the rest is C, and wherein said C₁₋₄alkyl may be optionally substituted with one or more substituents selected from fluorine and C₁₋₂alkoxy;
when Q is a pyrazolyl at least one of the N atoms in said pyrazolyl must be substituted with R11,
two R10 or one R10 and one R11 may, when sitting on neighbouring ring atoms and when both are represented by C₁₋₄alkyl be linked together by a carbon bond to form a fused ring system.

3. The compound according to claim 1, wherein Q is selected from optionally substituted thiophenyl, pyrrolyl, furanyl, oxazolyl, isoxazolyl, thiazolyl, isothiazolyl, pyrazolyl, [1,2,4]oxadiazolyl and [1,3,4]oxadiazolyl.

4. The compound according to claim 1, wherein R1 is selected from H, methyl, trifluoromethyl or difluoromethyl.

5. The compound according to claim 1, wherein four or more of R2, R3, R4, R5 and R6 are H.

6. The compound according to claim 1, wherein R2 is selected from H, methyl, trifluoromethyl, difluoromethyl, [2,2,2]-trifluoroethyl and cyano.

7. The compound according to claim 1, wherein each R10 is independently selected from C₁₋₄alkyl, C₁₋₂alkoxy, halogen and oxo, wherein said C₁₋₄alkyl is optionally substituted with one or more substituents selected from fluorine and C₁₋₂alkoxy.

8. The compound according to claim 1, wherein each R11 is independently selected from C₁₋₄alkyl and a monocyclic saturated ring moiety having 4-6 ring atoms, wherein one of said ring atoms may be O and the rest is C, and wherein said C₁₋₄alkyl may be optionally substituted with one or more substituents selected from fluorine and C₁₋₂alkoxy.

9. The compound according to claim 1 selected from
1: 3-methyl-N-[(2-methyl-1H-indol-5-yl)methyl]furan-2-carboxamide
2: 2,4-dimethyl-N-[(2-methyl-1H-indol-5-yl)methyl]thiazole-5-carboxamide
3: 5-Chloro-thiophene-2-carboxylic acid (2-methyl-1H-indol-5-ylmethyl)-amide
4: 3-chloro-N-[(2-methyl-1H-indol-5-yl)methyl]thiophene-2-carboxamide
5: N-[(2-methyl-1H-indol-5-yl)methyl]thiazole-4-carboxamide
6: N-[(2-methyl-1H-indol-5-yl)methyl]thiophene-3-carboxamide
7: 2,5-dimethyl-N-[(2-methyl-1H-indol-5-yl)methyl]furan-3-carboxamide
8: 5-methyl-N-[(2-methyl-1H-indol-5-yl)methyl]isoxazole-3-carboxamide
9: 3-methyl-N-[(2-methyl-1H-indol-5-yl)methyl]thiophene-2-carboxamide
10: N-[(2-methyl-1H-indol-5-yl)methyl]furan-2-carboxamide
11: N-[(2-methyl-1H-indol-5-yl)methyl]furan-3-carboxamide
12: 1,5-dimethyl-N-[(2-methyl-1H-indol-5-yl)methyl]pyrazole-3-carboxamide
13: N-[(2-methyl-1H-indol-5-yl)methyl]thiophene-2-carboxamide
14: N-[(2-methyl-1H-indol-5-yl)methyl]-1H-pyrrole-2-carboxamide
15: 4-methyl-N-[(2-methyl-1H-indol-5-yl)methyl]isoxazole-5-carboxamide
16: 4-methyl-N-[1-(2-methyl-1H-indol-5-yl)ethyl]isoxazole-5-carboxamide
17: 3-methyl-N-[(3-methyl-1H-indol-5-yl)methyl]isoxazole-5-carboxamide
18: 3-methoxy-N-[1-(2-methyl-1H-indol-5-yl)ethyl]isoxazole-5-carboxamide
19: 3-methoxy-N-[(2-methyl-1H-indol-5-yl)methyl]isoxazole-5-carboxamide
20: N-[(2-methyl-1H-indol-5-yl)methyl]thiazole-2-carboxamide
21: 2,4-dimethyl-N-[(2-methyl-1H-indol-5-yl)methyl]ioxazole-5-carboxamide
22: 4-methyl-N-[(2-methyl-1H-indol-5-yl)methyl]oxazole-5-carboxamide
23: 3-methyl-N-[(7-methyl-1H-indol-5-yl)methyl]isoxazole-5-carboxamide
24: 5-methyl-N-[(2-methyl-1H-indol-5-yl)methyl]thiophene-2-carboxamide
25: 2-methyl-N-[(2-methyl-1H-indol-5-yl)methyl]thiazole-4-carboxamide
26: N-[(2-methyl-1H-indol-5-yl)methyl]isoxazole-5-carboxamide
27: 3,5-dimethyl-N-[(2-methyl-1H-indol-5-yl)methyl]isoxazole-4-carboxamide
28: 3-methyl-N-[(2-methyl-1H-indol-5-yl)methyl]isoxazole-5-carboxamide
29: 1-methyl-N-[(2-methyl-1H-indol-5-yl)methyl]pyrazole-4-carboxamide
30: 1-methyl-N-[[2-(trifluoromethyl)-1H-indol-5-yl)methyl]pyrazole-4-carboxamide
31: N-[(2-(trifluoromethyl)-1H-indol-5-yl]methyl]isoxazole-5-carboxamide
32: 2-methyl-N-[(2-methyl-1H-indol-5-yl)methyl]-3-oxo-1H-pyrazole-5-carboxamide
33: 3-methyl-N-[1-(2-methyl-1H-indol-5-yl)ethyl]isoxazole-5-carboxamide
34: 3-isopropyl-N-[(2-methyl-1H-indol-5-yl)methyl]isoxazole-5-carboxamide
35: 2-methyl-N-[(2-methyl-1H-indol-5-yl)methyl]thiazole-5-carboxamide
36: N-[(2-methyl-1H-indol-5-yl)methyl]-1-(2,2,2-trifluoroethyl)pyrazole-4-carboxamide
37: 1-methyl-N-[(2-methyl-1H-indol-5-yl)methyl]pyrrole-3-carboxamide
38: N-[1-(1H-indol-5-yl)ethyl]-3-methyl-isoxazole-5-carboxamide
39: 3-methyl-N-[(6-methyl-1H-indol-5-yl)methyl]isoxazole-5-carboxamide
40: 1-(2,2,2-trifluoroethyl)-N-[[2-(trifluoromethyl)-1H-indol-5-yl]methyl]pyrazole-4-carboxamide
41: N-[(2,3-dimethyl-1H-indol-5-yl)methyl]-3-methyl-isoxazole-5-carboxamide
42: 2-methyl-N-[(1R)-1-(2-methyl-1H-indol-5-yl)ethyl]thiazole-5-carboxamide
43: 1-ethyl-N-[(1R)-1-(2-methyl-1H-indol-5-yl)ethyl]pyrazole-4-carboxamide
44: 1-methyl-N-[(1R)-1-(2-methyl-1H-indol-5-yl)ethyl]pyrazole-4-carboxamide
45: 3-methyl-N-[(1S)-2,2,2-trifluoro-1-(2-methyl-1H-indol-5-yl)ethyl]isoxazole-5-carboxamide
46: 1-(2-methoxyethyl)-N-[(1R)-1-(2-methyl-1H-indol-5-yl)ethyl]pyrazole-4-carboxamide
47: 3-methyl-N-[(1R)-1-(2-methyl-1H-indol-5-yl)ethyl]isothiazole-5-carboxamide
48: 1-(2-fluoroethyl)-N-[(1R)-1-(2-methyl-1H-indol-5-yl)ethyl]pyrazole-4-carboxamide
49: N-[(2-methyl-1H-indol-5H)methyl]-1-(oxetan-3-yl)pyrazole-4-carboxamide
50: 2-methyl-N-[(2-methyl-1H-indol-5-yl)methyl]oxazole-5-carboxamide
51: N-[(2-methyl-1H-indol-5-yl)methyl]-1-tetrahydrofuran-3-yl-pyrazole-4-carboxamide
52: N-[(1R)-1-(2-methyl-1H-indol-5-yl)ethyl]-1-(oxetan-3-yl)pyrazole-4-carboxamide
53: N-[(1R)-1-(4-fluoro-2-methyl-1H-indol-5-yl)ethyl]-3-methyl-isoxazole-5-carboxamide
54: N-[(1S)-1-(4-fluoro-2-methyl-1H-indol-5-yl)ethyl]-3-methyl-isoxazole-5-carboxamide
55: 2,5-dimethyl-N-[(2-methyl-1H-indol-5-yl)methyl]pyrazole-3-carboxamide
56: 4-chloro-3-methyl-N-[(2-methyl-1H-indol-5-yl)methyl]isoxazole-5-carboxamide
57: 1-methyl-N-[(2-methyl-1H-indol-5-yl)methyl]pyrrole-2-carboxamide
58: N-(1H-indol-5-ylmethyl)-3-methyl-isoxazole-5-carboxamide
59: N-(1H-indol-5-ylmethyl)-3,4-dimethyl-2-oxo-thiazole-5-carboxamide
60: 3,4-dimethyl-N-[(2-methyl-1H-indol-5-yl)methyl]-2-oxo-thiazole-5-carboxamide
61: 3-methyl-N-[(1R)-1-(2-methyl-1H-indol-5-yl)ethyl]isoxazole-5-carboxamide
62: 3-methyl-N-[(1S)-1-(2-methyl-1H-indol-5-yl)ethyl]isoxazole-5-carboxamide
63: N-[[1-(difluoromethyl)indol-5-yl]methyl]-3-methyl-isoxazole-5-carboxamide
64: N-[[1-(difluoromethyl)-2-methyl-indol-5-yl]methyl]-3-methyl-isoxazole-5-carboxamide
65: 3-methyl-N-[[2-(trifluoromethyl)-1H-indol-5-yl]methyl]isoxazole-5-carboxamide
66: 2-methyl-N-[[2-(trifluoromethyl)-1H-indol-5-yl]methyl]thiazole-5-carboxamide
67: 3-isopropyl-N-[[2-(trifluoromethyl)-1H-indol-5-yl]methyl]isoxazole-5-carboxamide
68: N-[[1-(difluoromethyl)indol-5-yl]methyl]-1-(2,2,2-trifluoroethyl)pyrazole-4-carboxamide
69: N-[[1-(difluoromethyl)-2-methyl-indol-5-yl]methyl]-1-(2,2,2-trifluoroethyl)pyrazole-4-carboxamide
70: N-[[1-(difluoromethyl)-2-methyl-indol-5-yl]methyl]-3-(2,2,2-trifluoroethyl)isoxazole-5-carboxamide
71: 1-isopropyl-N-[(2-methyl-1H-indol-5-yl)methyl]pyrazole-4-carboxamide
72: N-[[1-(difluoromethyl)indol-5-yl]methyl]-3-(2,2,2-trifluoroethyl)isoxazole-5-carboxamide
73: 3-methyl-N-[[2-(trideuteriomethyl)-1H-indol-5-yl]methyl]isoxazole-5-carboxamide
74: 5-methyl-N-[(2-methyl-1H-indol-5-yl)methyl]-1,3,4-oxadiazole-2-carboxamide
75: 5-ethyl-N-[(2-methyl-1H-indol-5-yl)methyl]isoxazole-3-carboxamide
76: 2-methyl-N-[(2-methyl-1H-indol-5-yl)methyl]ioxazole-4-carboxamide
77: 5-methyl-N-[(2-methyl-1H-indol-5-yl)methyl]-1,2,4-oxadiazole-3-carboxamide
78: N-[[2-(trideuteriomethyl)-1H-indol-5-yl]methyl]-3-(2,2,2-trifluoroethyl)isoxazole-5-carboxamide
79: N-[(2-methyl-1H-indol-5-yl)methyl]-3-(2,2,2-trifluoroethyl)isoxazole-5-carboxamide
80: 3-methyl-N-[[1-(trifluoromethyl)indol-5-yl]methyl]isoxazole-5-carboxamide
81: 1-(2,2,2-trifluoroethyl)-N-[[1-(trifluoromethyl)indol-5-yl]methyl]pyrazole-4-carboxamide
82: N-[[2-(trideuteromethyl)-1H-indol-5-yl]methyl]-1-(2,2,2-trifluoroethyl)pyrazole-4-carboxamide
83: N-[[2-methyl-1-(trifluoromethyl)indol-5-yl]methyl]-1-(2,2,2-trifluoroethyl)pyrazole-4-carboxamide
84: 3-methyl-N-[[2-methyl-1-(trifluoromethyl)indol-5-yl]methyl]isoxazole-5-carboxamide
85: N-[[2-methyl-1-(trifluoromethyl)indol-5-yl]methyl]-3-(2,2,2-trifluoroethyl)isoxazole-5-carboxamide
86: N-[(1R)-1-(2-methyl-1H-indol-5-yl)ethyl]-1-(2,2,2-trifluoroethyl)pyrazole-4-carboxamide
87: 5-tert-butyl-N-[(2-methyl-1H-indol-5-yl)methyl]isoxazole-3-carboxamide
88: N-[[1-methyl-2-(trifluoromethyl)indol-5-yl]methyl]-1-(2,2,2-trifluoroethyl)pyrazole-4-carboxamide
89: 3-methyl-N-[[1-methyl-2-(trifluoromethyl)indol-5-yl]methyl]isoxazole-5-carboxamide
90: N-[[1-methyl-2-(trifluoromethyl)indol-5-yl]methyl]-3-(2,2,2-trifluoroethyl)isoxazole-5-carboxamide
91: N-[[2-(difluoromethyl)-1H-indol-5-yl]methyl]-1-(2,2,2-trifluoroethyl)pyrazole-4-carboxamide
92: N-[[(2-(difluoromethyl)-1H-indol-5-yl]methyl]-3-methyl-isoxazole-5-carboxamide
93: N-[[2-(difluoromethyl)-1H-indol-5-yl]methyl]-3-(2,2,2-trifluoroethyl)isoxazole-5-carboxamide
94: 1-(2-fluoroethyl)-N-[(2-methyl-1H-indol-5-yl)methyl]pyrazole-3-carboxamide
95: 1-(2-methoxyethyl)-N-[(2-methyl-1H-indol-5-yl)methyl]pyrazole-3-carboxamide
96: 3-methyl-N-[1-[2-(trifluoromethyl)-1H-indol-5-yl]ethyl]isoxazole-5-carboxamide
97: 3-methyl-N-[1-[2-(trifluoromethyl)-1H-indol-5-yl]ethyl]isoxazole-5-carboxamide
98: 3-(methoxymethyl)-N-[(2-methyl-1H-indol-5-yl)methyl]isoxazole-5-carboxamide
99: 1-(2-fluoroethyl)-N-[(2-methyl-1H-indol-5-yl)methyl]pyrazole-4-carboxamide
100: 1-(2-methoxyethyl)-N-[(2-methyl-1H-indol-5-yl)methyl]pyrazole-4-carboxamide
101: N-[(2,3-dimethyl-1H-indol-5-yl)methyl]-1-(2,2,2-trifluoroethyl)pyrazole-4-carboxamide
102: N-[(1R)-1-(6-fluoro-2-methyl-1H-indol-5-yl)ethyl]-3-methyl-isoxazole-5-carboxamide
103: N-[(1R)-1-(6-fluoro-7-methyl-1H-indol-5-yl)ethyl]-3-methyl-isoxazole-5-carboxamide
104: N-[(6-fluoro-2-methyl-1H-indol-5-yl)methyl]-3-methyl-isoxazole-5-carboxamide
105: N-[(6-fluoro-7-methyl-1H-indol-5-yl)methyl]-3-methyl-isoxazole-5-carboxamide
106: 3-(2-fluoroethyl)-N-[(2-methyl-1,4-indol-5-yl)methyl]isoxazole-5-carboxamide
107: 3-methyl-N-[(2-methyl-1H-indol-5-yl)methyl]isothiazole-5-carboxamide
108: 3-methyl-N-[[2-(2,2,2-trifluoroethyl)-1H-indol-5-yl]methyl]isoxazole-5-carboxamide
109: N-[(7-fluoro-2-methyl-1H-indol-5-yl)methyl]-3-methyl-isoxazole-5-carboxamide
110: N-[(4-fluoro-2-methyl-1H-indol-5-yl)methyl]-3-methyl-isoxazole-5-carboxamide
111: N-[(1R)-1-(7-fluoro-2-methyl-1H-indol-5-yl)ethyl]-3-methyl-isoxazole-5-carboxamide
112: 3-(methoxymethyl)-N-[(1R)-1-(2-methyl-1H-indol-5-yl)ethyl]isoxazole-5-carboxamide
113: 3-(2-fluoroethyl)-N-[(1R)-1-(2-methyl-1H-indol-5-yl)ethyl]isoxazole-5-carboxamide
114: 3-(methoxymethyl)-N-[[2-(trifluoromethyl)-1H-indol-5-yl]methyl]isoxazole-5-carboxamide
115: 3-(2-fluoroethyl)-N-(1H-indol-5-ylmethyl)isoxazole-5-carboxamide
116: 5-methyl-N-[(2-methyl-1H-indol-5-yl)methyl]oxazole-2-carboxamide
117: N-[(2-methyl-1H-indol-5-yl)methyl]-5,6-dihydro-4H-pyrrolo[1,2-b]pyrazole-3-carboxamide
118: N-[(2-methyl-1H-indol-5-yl)methyl]-5,6-dihydro-4H-pyrrolo[1,2-b]pyrazole-2-carboxamide
119: 3-methyl-N-[(2-methyl-1H-indol-5-yl)methyl]-1,2,4-oxadiazole-5-carboxamide
120: N-[(2-cyano-1H-indol-5-yl)methyl]-3-methyl-isothiazole-5-carboxamide
121: N-[(1R)-1-(2-cyano-1H-indol-5-yl)ethyl]-3-methyl-isoxazole-5-carboxamide;
or a pharmaceutically acceptable salt of any of these compounds.

10. A compound according to claim 1 for use in therapy.

11. A compound according to claim 1 for use in the treatment of a disease or disorder selected from psychosis; schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PO); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.

12. The compound according to claim 11, wherein the treatment further comprises treatment with a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.

13. A pharmaceutical composition comprising a compound according to claim 1 and one or more pharmaceutically acceptable carriers or excipients.

14. The composition according to claim 13, which composition additionally comprises a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.

15. A kit comprising a compound according to claim 1 together with a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
